(19) United States
(12) Patent Application Publication
(10) Pub. No.: US 2016/0058502 A1
(43) Pub. Date: Mar. 3, 2016

(54) DEVICES FOR DAMAGING NERVES AND RELATED METHODS OF USE

(71) Applicant: Boston Scientific Scimed, Inc., Maple Grove, MN (US)

(72) Inventors: Bryan Allen CLARK, Forest Lake, MN (US); Aiden FLANAGAN, Kilcolgan (IE); Michael EPPIHIMER, Franklin, MA (US); Timothy A. OSTROOT, Cokato, MN (US); Derek C. SUTERMEISTER, Ham Lake, MN (US)

(21) Appl. No.: 14/833,585

(22) Filed: Aug. 24, 2015

Related U.S. Application Data
(60) Provisional application No. 62/144,689, filed on Apr. 8, 2015.
(60) Provisional application No. 62/042,659, filed on Aug. 27, 2014.

(51) Int. Cl. A61B 18/14

(52) U.S. Cl. CPC A61B 18/1492; A61B 2018/0022

(57) ABSTRACT

A method of treating an airway of a lung may include inserting a medical device into the airway, and delivering an agent from the medical device to a nerve disposed within or adjacent the airway to damage the nerve sufficient to reduce an ability of the nerve to send nerve signals.

### CROSS-REFERENCE TO RELATED APPLICATIONS

This patent application claims benefit of priority under 35 U.S.C. §119 to U.S. Provisional Patent Application No. 62/042,659, filed on Aug. 27, 2014, and U.S. Provisional Patent Application No. 62/144,689, filed on Apr. 8, 2015, the entireties of each of which are incorporated herein by reference.

### TECHNICAL FIELD

Various embodiments of the present disclosure relate generally to damaging nerves and related methods of use. More specifically, the present disclosure relates to devices, systems, and methods for damaging nerves within the lung.

### BACKGROUND

Chronic obstructive pulmonary disease (COPD) includes conditions such as, e.g., chronic bronchitis and emphysema. COPD currently affects over 15 million people in the United States alone and is currently the third leading cause of death in the country. The primary cause of COPD is the inhalation of cigarette smoke, responsible for over 90% of COPD cases. The economic and social burden of the disease is substantial and is increasing.

Chronic bronchitis is characterized by chronic cough with sputum production. Due to airway inflammation, mucus hypersecretion, airway hyperresponsiveness, and eventual fibrosis of the airway walls, significant airflow and gas exchange limitations result.

Emphysema is characterized by the destruction of the lung parenchyma. This destruction of the lung parenchyma leads to a loss of elastic recoil and tethering which maintains airway patency. Because bronchioles are not supported by cartilage like the larger airways, they have little intrinsic support and therefore are susceptible to collapse when destruction of tethering occurs, particularly during exhalation.

Acute exacerbations of COPD (AECOPD) often require emergency care and inpatient hospital care. An AECOPD is defined by a sudden worsening of symptoms (e.g., increase in or onset of cough, wheeze, and sputum changes) that typically last for several days, but can persist for weeks. An AECOPD is typically triggered by a bacterial infection, viral infection, or pollutants, which manifest quickly into airway inflammation, mucus hypersecretion, and bronchoconstriction, causing significant airway restriction.

Despite relatively efficacious drugs (long-acting muscarinic antagonists, long-acting beta agonists, corticosteroids, and antibiotics) that treat COPD symptoms, a particular segment of patients known as “frequent exacerbators” often visit the emergency room and hospital with exacerbations and also have a more rapid decline in lung function, poorer quality of life, and a greater mortality risk.

Reversible obstructive pulmonary disease includes asthma and reversible aspects of COPD. Asthma is a disease in which bronchoconstriction, excessive mucus production, and inflammation and swelling of airways occur, causing widespread but variable airflow obstruction thereby making it difficult for the asthma sufferer to breathe. Asthma is further characterized by acute episodes of airway narrowing via contraction of hyper-responsive airway smooth muscle.

The reversible aspects of COPD include excessive mucus production and partial airway occlusion, airway narrowing secondary to smooth muscle contraction, and bronchial wall edema and inflation of the airways. Usually, there is a general increase in bulk (hypertrophy) of the large bronchi and chronic inflammatory changes in the small airways. Excessive amounts of mucus are found in the airways, and semisolid plugs of mucus may occlude some small bronchi. Also, the small airways are narrowed and show inflammatory changes.

In asthma, chronic inflammatory processes in the airway play a central role in increasing the resistance to airflow within the lungs. Many cells and cellular elements are involved in the inflammatory process including, but not limited to, mast cells, eosinophils, T lymphocytes, neutrophils, epithelial cells, and even airway smooth muscle itself. The reactions of these cells result in an associated increase in sensitivity and hyperresponsiveness of the airway smooth muscle cells lining the airways to particular stimuli.

The chronic nature of asthma can also lead to remodeling of the airway wall (i.e., structural changes such as airway wall thickening or chronic edema) that can further affect the function of the airway wall and influence airway hyper-responsiveness. Epithelial denudation exposes the underlying tissue to substances that would not normally otherwise contact the underlying tissue, further reinforcing the cycle of cellular damage and inflammatory response.

In susceptible individuals, asthma symptoms include recurrent episodes of shortness of breath (dyspnea), wheezing, chest tightness, and cough. Currently, asthma is managed by a combination of stimulus avoidance and pharmacology.

The autonomic nervous system (ANS) provides constant control over airway smooth muscle, secretory cells, and vasculature. The ANS is divided into two subsystems, the parasympathetic nervous system and the sympathetic nervous system. These two systems operate independently for some functions, and cooperatively for other functions. The parasympathetic system is responsible for the unconscious regulation of internal organs and glands. In particular, the parasympathetic system is responsible for sexual arousal, salivation, lacrimation, urination, and digestion, among other functions. The sympathetic nervous system is responsible for stimulating activities associated with the fight-or-flight response. Although both sympathetic and parasympathetic branches of the ANS innervate lung airways, it is the parasympathetic branch that dominates with respect to control of airway smooth muscle, bronchial blood flow, and mucus secretions.

FIG. 1 illustrates the cholinergic control of airway smooth muscle and submucosal glands. An airway 100 may include an inner surface 102 that includes epithelial tissue 104. A nerve fiber 106 may include a plurality of receptors 108 that are disposed within epithelial tissue 104. Nerve fibers 106 may be C-fibers having receptors 108 disposed within epithelial tissue 104. Nerve fibers 106 may be afferent (sensory) nerves that carry nerve impulses from receptors 108 toward central nervous system (CNS) 109. Receptors 108 may respond to a wide variety of chemical stimuli and other irritants, such as, e.g., cigarette smoke, histamine, bradykinin, capsaicin, allergens, and pollens. C-fibers can also be triggered by autocoids that are released upon damage to tissues of the lung. The stimulation of receptors 108 by the various stimuli elicits reflex cholinergic bronchoconstriction.

Parasympathetic innervation of the airways is carried exclusively by vagus nerve 110 (e.g., the right and left vagus nerves). Upon receiving a signal from nerve fiber 106, CNS 109 may send a signal to initiate bronchoconstriction and/or mucus secretion. Cholinergic nerve fibers (e.g., nerve fibers that use acetylcholine (ACh) as their neurotransmitter) arise in the nucleus ambiguous in the brain stem and travel down a vagus nerve 110 (right and left vagus nerves) and synapse in parasympathetic ganglia 112 which are located within the airway wall. These parasympathetic ganglia are most numerous in the trachea and mainstem bronchi, especially near the hilus and points of bifurcations, with fewer ganglia that are smaller in size dispersed in distal airways. From these ganglia, short post-ganglionic fibers 114 travel to airway smooth muscle 116 and submucosal glands 118. ACh, the parasympathetic neurotransmitter, is released from post-ganglionic fibers and acts upon M1- and M3-receptors on smooth muscles 116 and submucosal glands 118 to cause bronchoconstriction (via constriction of smooth muscles 116), and the secretion of mucus 122 within airway 100 by submucosal glands 118, respectively. ACh may additionally regulate airway inflammation and airway remodeling, and may contribute significantly to the pathophysiology of obstructive airway diseases. Thus, fibers 114 may be efferent fibers (motor or effector neurons) that are configured to carry nerve impulses away from CNS 109.

FIG. 2 illustrates additional afferent nerve fibers located in airway 100 and in airway smooth muscle 116. Airway 100 may include one or more nerve fibers 106 and receptors 108 as described with reference to FIG. 1. Additionally, one or more nerve fibers 206 having one or more receptors 208 may be disposed within epithelial tissue 104. Nerve fibers 206 may be myelinated Rapidly Adapting Receptors (RAR) that respond to mechanical stimuli and are responsible in part for bronchoconstriction. Receptors 208 may respond to mechanical stimuli such as, e.g., water, airborne particulates, mucus, and the stretching of the lung during breathing or coughing. RARs may cause bronchoconstriction and are triggered by mechano-stimulation (e.g., mechanical pressure or distortion) and/or chemo-stimulation. Additionally, RARs may be triggered secondary to bronchoconstriction, leading to an amplification of the constriction response.

Airway smooth muscle 116 may be coupled to one or more receptors 210. Receptors 210 may be, e.g., Slowly Adapting Receptors (SARs) that are coupled to one or more nerve fibers 211.

Bronchial hyperresponsivity (BHR) may be present in a considerable number of COPD patients. Various reports have suggested BHR to be present in between ˜60% and 94% of COPD patients. This “hyperresponsivity” could be due to a “hyperreflexivity.” However there are several logical mechanisms by which parasympathetic drive may be overactivated in inflammatory disease. First, inflammation is commonly associated with overt activation and increases in excitability of vagal C-fibers in the airways that could increase reflex parasympathetic tone. Secondly, airway inflammation and inflammatory mediators have been found to increase synaptic efficacy and decrease action potential accommodation in bronchial parasympathetic ganglia, effects that would likely reduce their filtering function and lead to prolonged excitation. Thirdly, airway inflammation has also been found to inhibit muscarinic M2 receptor-mediated auto-inhibition of ACh release from postganglionic nerve terminals. This would lead to a larger end-organ response (e.g., smooth muscle contraction) per a given amount of action potential discharge. Fourthly, airway inflammation has been associated with phenotypic changes in the parasympathetic nervous system that could affect the balance of cholinergic contractile versus non-adrenergic non-cholinergic (NANC) relaxant innervation of smooth muscle.

Because airway resistance varies inversely with the fourth power of the airway radius, BHR is believed to be a function of both bronchoconstriction and inflammation. Inflammation in the airway walls reduces the inner diameter (or radius) of the airway lumen, thus amplifying the effect of even baseline cholinergic tone, because for a given change in muscle contraction, the airway lumen will close to a greater extent. BHR is likely caused by hypersensitivity of receptor nerve fibers, such as, e.g., C-fibers, RAR fibers, SAR fibers, and the like, lower thresholds for reflex action initiation, and reduced self-limitation of acetylcholine release.

The majority of vagal afferent nerves in the lungs are nociceptors that are adept at sensing the type of tissue injury and inflammation that occurs in the lungs in COPD. In addition, stretch sensitive afferent nerves are present in the lungs and can be activated by the tissue distention that occurs during eupneic (normal) breathing. The pattern of action potential discharge in these fibers depends on the rate and depth of breathing, the lung volume at which respiration is occurring, and the compliance of the lungs. Therefore, because COPD patients exhibit impaired breathing, the activity of nociceptive and mechano-sensitive afferent nerves is grossly altered in patients with COPD. The distortion in vagal afferent nerve activity in COPD may lead to situations where these responses are out of sync with the body's needs.

Thus, a need exists for patients suffering from diseases of the lung. In some embodiments, normalizing the activity in bronchopulmonary vagal afferent nerves may limit symptoms that accompany various diseases of the lung.

### SUMMARY OF THE DISCLOSURE

The present disclosure includes devices for damaging nerves and related methods of use.

In one aspect the present disclosure is directed to a method of treating an airway of a lung. The method may include inserting a medical device into the airway, and delivering an agent from the medical device to a nerve disposed within or adjacent the airway to damage the nerve sufficient to reduce an ability of the nerve to send nerve signals.

Various examples of the present disclosure may include one or more of the following features: wherein the agent is a neurolytic agent and includes one or more of ethanol, phenol, glycerol, ammonium salt compounds, chlorocresol and hypertonic and/or hypotonic solutions; wherein the nerve is located within a branch point of two or more lung airways; wherein the branch point is a carina; wherein only nerves disposed at or adjacent to branch points of lung airways are damaged; wherein the medical device includes an inflatable member having a plurality of pores, and the agent is delivered through the pores; wherein the medical device includes a first inflatable member movable between a deflated configuration and an inflated configuration, an outer surface of the first inflatable member further including a needle configured to deliver agent through the airway wall toward the nerve while the first inflatable member is in the inflated configuration; wherein the medical device further includes an expandable member movable between a first configuration and a second configuration, the expandable member being configured to anchor the medical device to the airway wall while in the second configuration, and the method further includes moving the expandable member to the second configuration before moving the first inflatable member into the inflated configuration; wherein the medical device has a deployment member with at least one fluid delivery device, and delivering the agent includes moving the medical device along a longitudinal axis of the medical device to cause the deployment member and the at least one fluid delivery device to rotate; wherein delivering agent includes rotating a fluid delivery device to coat the agent on an airway wall; wherein the agent is disposed within dissolvable strips that are delivered to the airway wall; wherein the dissolvable strips are formed in a spiral, circumferential, or axial shape; wherein the medical device includes a proximal expandable member, a distal expandable member, and a plurality of openings disposed between the proximal and distal expandable members, and the agent is delivered through at least one of the plurality of openings into a sealed region formed by the airway and the proximal and distal expandable members; further including applying a rinsing substance through at least one of the plurality of openings, and evacuating residual agent and rinsing substance through one or more of the plurality of openings; wherein the medical device further includes an applicator formed from a plurality of porous fibers, wherein the agent is delivered to the surface of the airway wall via the applicator; wherein the medical device includes an elongate member and a plurality of delivery devices disposed within the elongate member, the plurality of delivery devices being movable between a first configuration and a second configuration, wherein the plurality of delivery devices are constrained within the elongate member in the first configuration and disposed distal to and radially outward from the elongate member in the second configuration to deliver the agent through the airway wall; wherein a distal end of each fluid delivery device has a stop configured to limit a depth of penetration of each respective fluid delivery device into the airway wall; wherein the medical device includes an elongate member having a bend such that a distal end of the elongate member is offset from a longitudinal axis of the medical device, and a fluid delivery device extending distally from the distal end of the elongate member; further including an expandable member on the elongate member, the expandable member configured to orient the medical device closer toward one radial side of the airway than an opposing radial side of the airway; wherein the medical device includes an expandable member and one or more fluid delivery devices coupled to the expandable member, wherein delivering an agent from the medical device to the nerve further includes expanding the expandable member to move the fluid delivery devices to a position offset from the longitudinal axis of the medical device and through a wall of the airway; further including identifying the nerve, penetrating a surface of the airway with a delivery device, and delivering the agent directly to the nerve; and wherein the agent damages only a targeted nerve disposed within or adjacent to the airway; wherein the targeted nerve is an afferent sensory nerve; wherein the targeted nerve is a C-fiber; wherein the agent is capsaicin.

In another aspect, the present disclosure is directed to a medical device for treating a lung. The medical device may include an elongate member, and a proximal expandable member disposed on a distal end of the elongate member. The medical device may also include a distal expandable member disposed distal to the proximal expandable member; and a plurality of openings disposed on the elongate member between the proximal and distal expandable members. The medical device may be configured to deliver an agent through at least one of the plurality of openings into a sealed region formed by a lung airway and the proximal and distal expandable members.

In yet another aspect, the present disclosure is directed to a medical device for treating a lung. The medical device may include a first elongate member, and a second elongate member extending from the first elongate member, the second elongate member having a bend such that a distal end of the second elongate member is offset from a longitudinal axis of the medical device. The medical device may also include a fluid delivery device extending distally from the distal end of the second elongate member, and an expandable member disposed on at least one of the first and second elongate members. The expandable member may be configured to orient the fluid delivery closer toward one radial side of a lung airway than an opposing radial side of the lung airway.

In yet another aspect, the present disclosure is directed to a medical device for treating a lung. The medical device may include a first elongate member, and one or more second elongate members extending from the first elongate member, each of the one or more second elongate members including a fluid delivery device extending from a distal end. The medical device may also include a third elongate member extending from the first elongate member, the third elongate member having an expandable member. Expanding the expandable member from a collapsed configuration to an expanded configuration may move the distal ends of the one or more second elongate members to a position offset from the longitudinal axis of the medical device.

In yet another aspect, the present disclosure is directed to a method of treating an airway of a lung. The method may include inserting a medical device into the airway, and applying optical energy from the medical device to a nerve disposed within or adjacent the airway to damage the nerve sufficient to reduce an ability of the nerve to send nerve signals.

Various examples of the present disclosure may include one or more of the following features: wherein the nerve is located within a branch point of two or more lung airways; wherein the branch point is a carina; wherein only nerves disposed at or adjacent to branch points of lung airways are damaged; wherein the medical device includes an elongate member, and the optical energy is delivered from the elongate member; wherein the optical energy is directed distally from a distal end of the elongate member; wherein the optical energy is directed toward the lung airway from a plurality of longitudinally spaced locations along the elongate member; wherein the optical energy is emitted from a plurality of locations along the elongate member, and focused toward one target treatment area from the plurality of locations along the elongate member; wherein the optical energy has an annular cross-section; wherein the optical energy is focused at a surface of the lung airway; wherein the optical energy is focused at a depth beyond the surface of the lung airway; wherein the elongate member includes a rotating portion, and the optical energy is delivered from the rotating portion to the lung airway; wherein the medical device further includes a guide member extending from the elongate member toward a surface of the lung airway, the method further including delivering the optical energy through the guide member; wherein the medical device further includes one or more biased guide members extending from the elongate member, the one or more biased guide members being configured to locate the medical device at a middle portion of the lung airway; wherein the medical device includes an expandable member having a plurality of legs and at least one optical element disposed on a partial-length of at least one of the plurality of legs such that a distal end of the at least one optical element is directed toward a wall of the airway at an angle offset from a longitudinal axis of the medical device; wherein the medical device includes a conical expandable member, and at least one optical element is coupled to the conical expandable member such that a distal end of the at least one optical element is directed toward a wall of the airway at an angle offset from a longitudinal axis of the medical device; herein the medical device includes an expandable member having a plurality of legs, a central elongate member extending through the expandable member to a distal tip of the expandable member, and at least one optical element disposed on central elongate member such that a distal end of the at least one optical element is directed toward a wall of the airway at an angle offset from a longitudinal axis of the medical device; wherein the optical energy is applied to a nerve and has a therapeutically negligible effect on non-nerve tissue; and further including delivering a probe to the lung that is configured to specifically bind to the nerve, and applying optical energy at a wavelength to excite the probe, wherein the optical energy has a therapeutically negligible effect on tissues that are not bound to the probe.

In yet another aspect, the present disclosure is directed to a method of treating an airway of a lung. The method may include inserting a medical device into the airway, and utilizing the medical device to reduce a temperature of a nerve disposed within or adjacent the airway sufficient to damage the nerve sufficient to reduce an ability of the nerve to send nerve signals.

Various examples of the present disclosure may include one or more of the following features: wherein the nerve is located within a branch point of two or more lung airways; wherein the branch point is a carina; wherein only nerves disposed at or adjacent to branch points of lung airways are damaged; wherein the medical device further includes an elongate member and a cooling member disposed at a distal end of the elongate member, the cooling member being configured to reduce the temperature of the nerve disposed within or adjacent the airway; further including passing a cooled substance through the cooling member to reduce the temperature of the nerve disposed within or adjacent the airway; wherein the cooling member is an inflatable balloon, and the inflatable balloon further includes a raised portion, and the method further includes contacting a surface of the airway wall with only the raised portion; wherein the cooling member is an inflatable balloon, and the inflatable balloon further includes at least one active region and at least one insulated region each disposed on an outer surface of the inflatable balloon, wherein the active region is configured to reduce a temperature of airway tissue upon contact with airway tissue, and the insulated region is configured to have substantially less therapeutic effect on airway tissue temperature upon contact with airway tissue; wherein the medical device is further configured to deliver a cryospray to reduce the temperature of the nerve disposed within or adjacent the airway; wherein the cryospray is delivered from at least one opening disposed along the medical device, and the medical device further includes an expandable member disposed distal to the at least one opening; further including sensing a control temperature indicative of the temperature of the nerve, and altering a deployment parameter of the cryospray based on the sensed control temperature; further including moving the medical device proximally while simultaneously applying the cryospray, and wherein the deployment parameter is a speed of the medical device moving proximally through the airway; wherein, when the sensed control temperature is below a treatment threshold temperature, the speed of the medical device is increased; wherein, when the sensed control temperature is above a treatment threshold temperature, the speed of the medical device is decreased; wherein the cryospray is applied to a proximal airway of a patient, and the control temperature is sensed at a distal airway of the patient; wherein the control temperature is sensed by a sensing element disposed through an airway wall and in contact with the nerve; wherein the medical device further includes an elongate member having one or more active regions disposed on the elongate member and an expandable member configured to position the one or more active regions of the elongate member against a wall of the airway, the one or more active regions being configured to reduce the temperature of the airway; wherein the medical device further includes an elongate member having one or more active regions disposed on the elongate member, and the elongate member is configured to have a spiral, sinusoidal, or wavy configuration when in an expanded configuration such that the one or more active regions delivers a spiral, sinusoidal, or wavy treatment pattern along a wall of the airway.

In yet another aspect, the present disclosure is directed to a medical device for treating a lung. The medical device may include an elongate member, and a proximal expandable member disposed on a distal end of the elongate member. The medical device may also include a distal expandable member disposed distal to the proximal expandable member, and a plurality of openings disposed on the elongate member between the proximal and distal expandable members, wherein the medical device is configured to deliver a cryospray through at least one of the plurality of openings into a sealed region formed by a lung airway and the proximal and distal expandable members.

In yet another aspect, the present disclosure is directed to a medical device for treating a lung. The medical device may include an elongate member configured to deliver a cryospray to a first airway of the lung, and a temperature sensing element configured to measure a temperature of a second airway of the lung that is distal to the first airway. The medical device may also include a controller coupled to the elongate member and to the temperature sensing element. The controller may be configured to vary a deployment parameter of cryospray delivery based on the temperature sensed by the temperature sensing element at the second airway.

In yet another aspect, the present disclosure is directed to a medical device for treating a lung. The medical device may include an elongate member having one or more active regions disposed on the elongate member, the active regions being configured to contact the airway to reduce a temperature of the airway. The elongate member may be formed in a spiral, sinusoidal, or wavy configuration when in an expanded configuration such that the one or more active regions reduces the temperature of the airway in a spiral, sinusoidal, or wavy treatment pattern.

In yet another aspect, the present disclosure is directed to a method of treating an airway of a lung. The method may include inserting a medical device into the airway, and applying acoustic energy from the medical device to a nerve disposed within or adjacent the airway to damage the nerve sufficient to reduce an ability of the nerve to send nerve signals.

Various examples of the present disclosure may include one or more of the following aspects: wherein the nerve is located within a branch point of two or more lung airways; wherein the branch point is a carina; wherein only nerves disposed at or adjacent to branch points of lung airways are damaged; wherein the acoustic energy is in the frequency range of 20 kHz to 20 MHz; further including anchoring the medical device within the airway via an anchoring member; wherein the anchoring member is an expandable basket having a plurality of legs disposed around a longitudinal axis of the medical device; wherein the acoustic energy is delivered from an energy delivery element disposed on at least one of the plurality of legs; wherein the anchoring member is an inflatable balloon; wherein the acoustic energy is delivered from an energy delivery element disposed within the balloon; wherein the acoustic energy is delivered from an energy delivery element disposed on an outer surface of the balloon; wherein the acoustic energy is delivered from an energy delivery element disposed within a groove defined by an outer surface of the balloon; wherein the balloon further includes a plurality of pores, and the method further includes applying a fluid to the airway via the pores to improve the transmission of acoustic energy; wherein the balloon includes a proximal portion and a plurality of branches extending from the proximal portion, and acoustic energy is delivered to a plurality of airways via energy delivery elements disposed within each of the proximal portion and the plurality of branches; further including inflating the balloon with a cryofluid to cool the lung airways and reduce damage to a surface of the lung airways; wherein the medical device further includes an elongate member having one or more energy delivery elements disposed on the elongate member and an expandable member configured to position the one or more energy delivery elements of the elongate member against a wall of the airway, the one or more energy delivery elements being configured to transmit acoustic energy; wherein the medical device further includes an elongate member having one or more energy delivery elements disposed on the elongate member, and the elongate member is configured to have a spiral, sinusoidal, or wavy configuration when in an expanded configuration such that the one or more energy delivery elements delivers a spiral, sinusoidal, or wavy treatment pattern along a wall of the airway; wherein the medical device includes a spiral track at a distal end, and the energy delivery element is configured to travel along the spiral track; wherein the medical device further includes a first expandable member distal to the spiral track, and a second expandable member proximal to the spiral track; wherein the first and second expandable members serve as distal and proximal stops as the energy delivery element travels along the spiral track.

In yet another aspect the present disclosure is directed to a medical device for treating a lung. The medical device may include an elongate member having a first branch point at a distal end of the elongate member, and two or more linking members extending from the first branch point. The medical device may also include a first energy delivery element disposed proximal to the first branch point, one or more second energy delivery elements disposed on each of the two or more linking members, the first and second energy delivery elements being configured to deliver acoustic energy to an airway of the lung. The medical device may also include an inflatable member covering the two or more linking members, the first energy delivery element, and the one or more second energy delivery elements, and the inflatable member having a second branch point configured to abut a branch point of two or more lung airways such that each of the two or more linking members extends through one of the lung airways.

In yet another aspect, the present disclosure is directed to a medical device for treating a lung. The medical device may include an elongate member having one or more energy delivery elements disposed on the elongate member, the energy delivery elements being configured to apply acoustic energy to the airway sufficient to damage a nerve disposed within the airway. The elongate member may be formed in a spiral, sinusoidal, or wavy configuration when in an expanded configuration such that the one or more energy delivery elements delivers acoustic energy in spiral, sinusoidal, or wavy treatment pattern along a wall of the airway.

In yet another aspect, the present disclosure is directed to a medical device for treating tissue. The medical device may include an expandable member configured to be disposed in a body lumen and move between a collapsed configuration and an expanded configuration via the delivery of a fluid to the expandable member. The expandable member may permit the passage of substances through the body lumen and distally of the expandable member while the expandable member is in the expanded configuration. The medical device also may include one or more energy delivery elements coupled to the expandable member. The one or more energy delivery elements may be configured to damage tissues disposed radially outward of the lumen.

The expandable member may include a plurality of radially outermost portions that are configured to cause one or more energy delivery elements to contact tissue when the expandable member is in the expanded configuration, wherein the plurality of radially outermost portions are radially spaced from one another. The one or more energy delivery elements may be disposed on the plurality of radially outermost portions. The expandable member may have a x-shaped cross-section. The expandable member may include an inner lumen that permits the passage of substances disposed within the body lumen through the expandable member when the expandable member is in the expanded configuration. The expandable member may have a ring-shaped cross-section. An outer surface of the expandable member may be configured to contact an entire circumference of a body lumen while in the expanded configuration.

In yet another aspect, the present disclosure may be directed to a medical device for treating tissue. The medical device may include an expandable member configured to move between a collapsed configuration and an expanded configuration. The expandable member may include a plurality of expandable legs that each extend from a proximal end toward a distal end in a spiral configuration. Each of the expandable legs may have one or more radially outermost portions. Each of the plurality of expandable legs may extend along a different trajectory such that each of the radially outermost portions of the medical device may be longitudinally and radially staggered from one another. The medical device also may include a plurality of energy delivery elements coupled to the expandable member and configured to damage tissues disposed radially outward of the lumen. Each of the plurality of energy delivery elements may be positioned at a radially outermost portion of a corresponding one of the plurality of expandable legs.

The distal ends of the plurality of expandable legs may converge toward one another at a distal end. The distal ends of the plurality of expandable legs may be unconnected to one another.

In yet another aspect, the present disclosure may be directed to a medical device for treating tissue. The medical device may include an expandable member configured to move between a collapsed configuration, a partially-expanded configuration, and an expanded configuration via the delivery of a fluid to the expandable member. The medical device also may include an energy delivery element coupled to the expandable member. The energy delivery element may include a blunt tip configured to apply a pressure to the surface of tissue when the medical device is in the expanded configuration. The energy delivery element may be disposed on an outer surface of the expandable member at a first portion. The first portion of the outer surface may be disposed closer to a radial center of the expandable member than a remaining portion of the outer surface when the expandable member is in the partially-expanded configuration.

The energy delivery element may include two inclined edges that meet at a bladed tip. Each of the inclined edges may include an outlet in fluid communication with an interior of the expandable member. The bladed tip may include an outlet in fluid communication with an interior of the expandable member.

In yet another aspect, the present disclosure may be directed to a medical device for treating tissue. The medical device may include a first RF energy delivery element configured to pierce through tissue, and a second RF energy delivery element configured to pierce through tissue. The second RF energy delivery element may be radially spaced from the first RF energy delivery element. The medical device may be configured to deliver RF energy in a bipolar manner between the first and second RF energy delivery elements.

Each of the first and second RF energy delivery elements may include a proximal portion and a distal portion extending from the proximal portion, wherein both the proximal portion and the distal portion are configured to pierce through tissue, and wherein the proximal portion is electrically insulated and the distal portion is electrically active.

In yet another aspect, the present disclosure is directed to a method of treating an airway of a lung. The method may include inserting a medical device into the airway, and applying microwave energy from an energy delivery element of the medical device to at least one of: a nerve disposed within or adjacent the airway to damage the nerve sufficient to reduce an ability of the nerve to send nerve signals, and a tumor.

The energy delivery element may be disposed within an expandable member. The expandable member may be a balloon. The method may further include circulating a cooling fluid through the balloon during energy delivery. The method may further include at least partially blocking microwave energy emitted by the energy delivery element. The medical device may further include an expandable member configured to absorb microwaves. The expandable member may be disposed either proximal to or distal to the energy delivery element. The method may further include delivering a substance to the nerve or tumor that is configured to absorb microwave energy at a faster rate than surrounding tissue. The substance may be hypertonic saline. The microwave energy may be applied at a rate that is only sufficient to ablate the nerve or tumor when the substance is applied, and tissues surrounding the nerve or tumor are not ablated.

In yet another aspect, the present disclosure may be directed to a medical device for treating tissue. The medical device may include an expandable member configured to be disposed in a body lumen and move between a collapsed configuration and an expanded configuration via the delivery of a fluid to the expandable member. The expandable member may permit the passage of substances through the body lumen from proximal of the expandable member to distally of the expandable member while the expandable member is in the expanded configuration. The medical device may include one or more energy delivery elements coupled to the expandable member, the one or more energy delivery elements being configured to damage tissues disposed radially outward of the lumen.

The expandable member may include a plurality of radially outermost portions that are configured to cause one or more energy delivery elements to contact tissue when the expandable member is in the expanded configuration, wherein the plurality of radially outermost portions are circumferentially spaced from one another. The one or more energy delivery elements may be disposed on the plurality of radially outermost portions. Each of the plurality of radially outermost portions may include an energy delivery element. The medical device may include a plurality of energy delivery elements arranged in a spiral about the plurality of radially outermost portions. The expandable member may have a x-shaped cross-section. The expandable member may include an inner lumen that permits the passage of substances disposed within the body lumen through the expandable member when the expandable member is in the expanded configuration. The expandable member may have a ring-shaped cross-section. An outer surface of the expandable member may be configured to contact an entire circumference of a body lumen while in the expanded configuration. The expandable member may be a balloon.

In yet another aspect, the present disclosure may be directed to a medical device for treating tissue. The medical device may include an expandable member configured to move between a collapsed configuration and an expanded configuration, the expandable member including a plurality of expandable legs that each extends from a proximal end toward a distal end in a spiral configuration, each of the expandable legs having one or more radially outermost portions, wherein each of the plurality of expandable legs extends along a different trajectory such that each of the radially outermost portions of the medical device are longitudinally and circumferentially staggered from one another. The medical device may also include a plurality of energy delivery elements coupled to the expandable member and configured to damage tissues disposed radially outward of the lumen, wherein each of the plurality of energy delivery elements is positioned at a radially outermost portion of a corresponding one of the plurality of expandable legs.

The distal ends of the plurality of expandable legs may converge toward one another at a distal end. The distal ends of the plurality of expandable legs may be unconnected to one another. Each of the plurality of energy delivery elements may include a lumen, and wherein the medical device may be configured to circulate a cooling fluid through the lumen of each of the plurality of energy delivery elements.

In yet another aspect, the present disclosure may be directed to a medical device for treating tissue. The medical device may include an expandable member configured to move between a collapsed configuration, a partially-expanded configuration, and an expanded configuration via the delivery of a fluid to the expandable member. The medical device also may include an energy delivery element coupled to the expandable member, the energy delivery element including a bladed tip configured to apply a pressure to the surface of tissue when the medical device is in the expanded configuration, wherein the energy delivery element is disposed on an outer surface of the expandable member at a first portion, wherein the first portion of the outer surface is disposed closer to a radial center of the expandable member than a remaining portion of the outer surface when the expandable member is in the partially-expanded configuration.

The energy delivery element may include two inclined surfaces that meet at the bladed tip. Each of the inclined surfaces may include an outlet in fluid communication with an interior of the expandable member. The bladed tip may include an outlet in fluid communication with an interior of the expandable member. The expandable member may be a balloon. The energy delivery element may be configured to deliver radiofrequency energy.

### BRIEF DESCRIPTION OF THE FIGURES

The accompanying drawings, which are incorporated in and constitute a part of this specification, illustrate various exemplary embodiments and together with the description, serve to explain the principles of the disclosed embodiments.

FIG. 1 is a schematic view of an airway and a cholinergic pathway.

FIG. 2 is a schematic view of an airway and afferent nerves.

FIG. 3 is a schematic view of the lungs being treated with a treatment device according to an embodiment of the present disclosure.

FIG. 4 is a cross-sectional view of an airway in a healthy lung.

FIG. 5 is a schematic view of the trachea and lung airways.

FIGS. 6-9 illustrate treatment procedures in accordance with various embodiments of the present disclosure.

FIG. 10 is a partial side view of a medical device in a retracted configuration in accordance with an embodiment of the present disclosure.

FIG. 11 is a partial side view of the medical device of FIG. 10 in a partially-retracted, partially expanded configuration in accordance with an embodiment of the present disclosure.

FIG. 12 is a partial side view of the medical device of FIG. 10 in an expanded configuration.

FIG. 13 is a partial side view of a medical device in a retracted configuration in accordance with another embodiment of the present disclosure.

FIG. 14 is a partial side view of the medical device of FIG. 13 in an expanded configuration.

FIG. 15 is a partial perspective view of a medical device in accordance with an embodiment of the present disclosure.

FIG. 16 is a partial perspective view of a medical device in accordance with another embodiment of the present disclosure.

FIG. 17 is a partial side view of a medical device in a retracted configuration in accordance with another embodiment of the present disclosure.

FIG. 18 is a partial side view of the medical device of FIG. 17 in an expanded configuration.

FIG. 19 is a partial side view of a medical device in an expanded configuration in accordance with another embodiment of the present disclosure.

FIG. 20 is a partial side view of a medical device in a first configuration in accordance with another embodiment of the present disclosure.

FIG. 21 is a partial side view of the medical device of FIG. 20 in a second configuration.

FIG. 22 is a partial side view of the medical device of FIG. 20 in a third configuration.

FIGS. 23-25 are partial side views of medical devices for delivering a dissolvable neurolytic agent in accordance with various embodiments of the present disclosure.

FIG. 26 is an in vivo illustration of a medical device for applying neurolytic agent in accordance with another embodiment of the present disclosure.

FIG. 27 is an in vivo illustration of a medical device for applying neurolytic agent in accordance with another embodiment of the present disclosure.

FIG. 28 is a partial side view of a medical device for applying neurolytic agent in accordance with another embodiment of the present disclosure.

FIGS. 29-36 are partial side views of medical devices according to various embodiments of the present disclosure.

FIG. 37 is a cross-sectional view of the medical device of FIG. 36 taken along line 37-37.

FIGS. 38-49 are partial side views of medical devices, according to various embodiments of the present disclosure.

FIG. 50 is a cross-sectional view of the medical device of FIG. 49 taken along line 49-49.

FIGS. 51-53 are side views of energy delivery devices, according to various embodiments of the present disclosure.

FIGS. 54-56 are schematic illustrations of treatment patterns along an airway, according to various embodiments of the present disclosure.

FIGS. 57-63 are side views of various energy delivery devices, according to various embodiments of the present disclosure.

FIG. 64 is an in vivo illustration of a medical device, according to one embodiment of the present disclosure.

FIG. 65 is an in vivo illustration of a medical device, according to another embodiment of the present disclosure.

FIG. 66 is an in vivo illustration of a medical device, according to yet another embodiment of the present disclosure.

FIGS. 67 and 68 are partial side views of medical devices according to various embodiments of the present disclosure.

FIGS. 69-75 are perspective views of medical devices, according to various embodiments of the present disclosure.

FIG. 76 is a cross-sectional view of the medical device of FIG. 75 taken along line 76-76.

FIG. 77 is a perspective view of a medical device, according to an embodiment of the present disclosure.

FIG. 78 is a cross-sectional view of the medical device of FIG. 77 taken along line 78-78.

FIG. 79 is a perspective view of a medical device in a collapsed configuration, according to an embodiment of the present disclosure.

FIG. 80 is a perspective view of the medical device of FIG. 790 in an expanded configuration.

FIG. 81 is a side view of a medical device, according to an embodiment of the present disclosure.

FIG. 82 is a partial side view of a medical device, according to an embodiment of the present disclosure.

FIG. 83 is a partial side view of a medical device, according to another embodiment of the present disclosure.

FIG. 84 is a cross-sectional view of the medical device of FIG. 83, taken along line 84-84.

FIG. 85 is a partial side view of a medical device, according to another embodiment of the present disclosure.

FIG. 86 is a cross-sectional view of the medical device of FIG. 85, taken along line 86-86.

FIGS. 87-89 are partial side views of medical devices, according to embodiments of the present disclosure.

FIG. 90 is an enlarged view of section A of the medical device of FIG. 89.

FIG. 91 is a partial side view of a medical device in a collapsed configuration.

FIG. 92 is a partial side view of a medical device in an expanded configuration.

FIG. 93 is an end view of a medical device in a collapsed configuration, according to another embodiment of the present disclosure.

FIG. 93A is a partial side view of the medical device of FIG. 93 while in a partially expanded configuration.

FIG. 94 is a cross-sectional view of the medical device of FIG. 93A, taken along line 94-94.

FIGS. 95-98 depict various energy delivery elements according to various embodiments of the present disclosure.

FIGS. 99-100 are partial side view of various medical devices, according to embodiments of the present disclosure.

FIGS. 101-102 are end views of various medical devices, according to embodiments of the present disclosure.

FIGS. 103-106 are partial side view of various medical devices, according to embodiments of the present disclosure.

### DETAILED DESCRIPTION

Reference will now be made in detail to embodiments of the present disclosure, examples of which are illustrated in the accompanying drawings. Wherever possible, the same reference numbers will be used throughout the drawings to refer to the same or like parts.

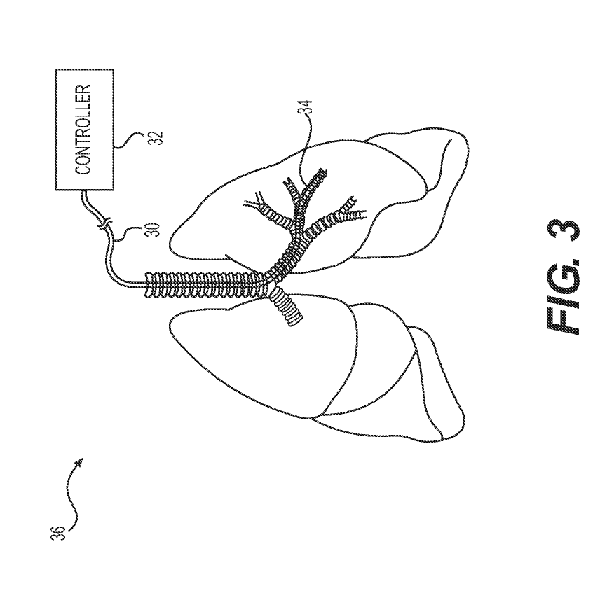

FIG. 3 is an illustration of a lung being treated with a system 36 according to the present invention. The system 36 may include a controller 32 and a delivery device 30 which may be an elongated member as described further below. The delivery device 30 may include a tool that can be positioned at a treatment site 34 within a lung or another target medium.

In some embodiments, the controller 32 may include a processor that is generally configured to accept information from the system and system components, and process the information according to various algorithms to produce control signals for controlling the delivery device 30. The processor may accept information from the system and system components, process the information according to various algorithms, and produce information signals that may be directed to visual indicators, digital displays, audio tone generators, or other indicators of, e.g., a user interface, in order to inform a user of the system status, component status, procedure status or any other useful information that is being monitored by the system. The processor may be a digital IC processor, analog processor or any other suitable logic or control system that carries out the control algorithms.

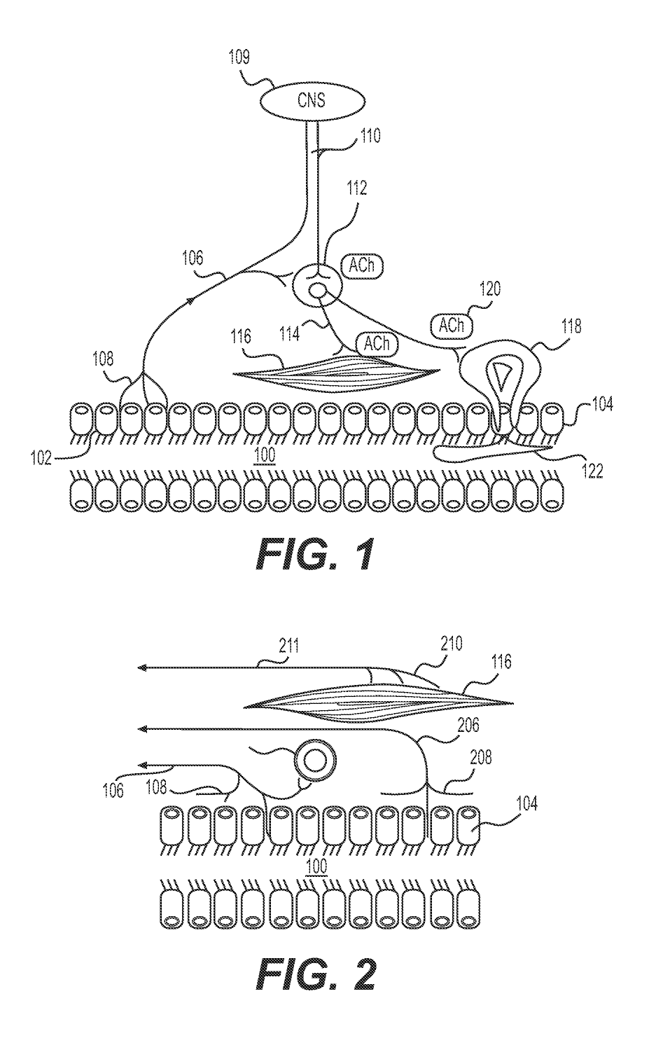

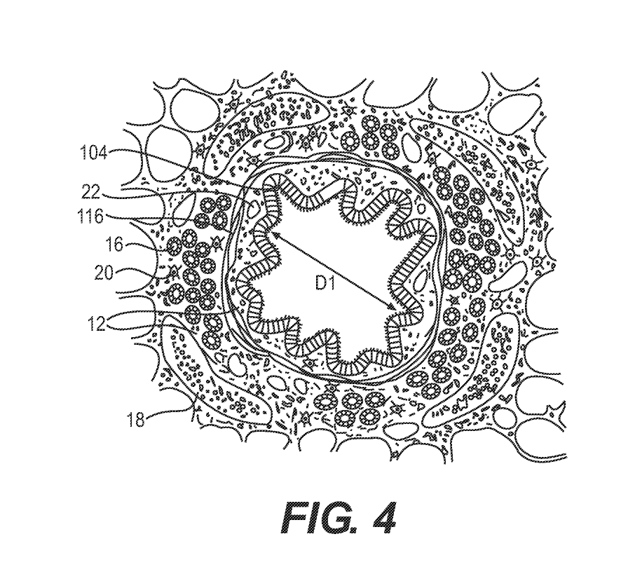

FIG. 4 illustrates a cross-section of an airway in a healthy patient. The airway of FIG. 2 may be a medium-sized bronchus having an airway diameter D1 of about 3 mm, although the airway may have another suitable diameter. The airway may include a folded inner surface or epithelial tissue 104 surrounded by stroma 12 and smooth muscle tissue 116. Epithelial tissue 104 may include afferent sensory nerves, among other nerves. The larger airways including the bronchus shown in FIG. 1 may have mucous glands 16 and cartilage 18 surrounding the smooth muscle tissue 14. Nerve fibers 20 and blood vessels 22 may surround the airway. Nerve fibers 20 may include, e.g., both afferent and efferent nerves.

Denervation

In some embodiments, therapy delivered by medical devices of the present disclosure may reduce acute exacerbations in COPD patients through the reduction of bronchoconstriction and mucus secretion caused by parasympathetic nerve activity. In other embodiments, symptoms of asthma, cystic fibrosis, chronic cough, or other diseases of the lung may be reduced or eliminated. Additionally, a reduction in airway inflammation and remodeling may be achieved. In some embodiments, the therapy may result in a reduction in the release of acetylcholine (ACh) or inflammatory mediators (e.g., tachykinins) from nerves in the airways of the lung. Thus, less ACh may be available to bind to muscarinic M1- and M3-receptors on smooth muscle cells and submucosal glands in the lung, resulting in less bronchoconstriction and mucus production.

The embodiments of the present disclosure may impair the transmission of signals from nerves (e.g., afferent receptors, afferent fibers, efferent nerve cell bodies, efferent nerve trunks, efferent fibers, C-Fibers, RAR fibers, SAR fibers, or the like) in the epithelium or airway walls which evoke reflex bronchoconstriction responses when activated by irritants or stimulants. Stimulation of these nerves may evoke bronchoconstriction, mucus production, cough, and pulmonary edema through either pre-ganglionic parasympathetic activity (acting on the central nervous system) or post-ganglionic parasympathetic activity (acting directly on parasympathetic ganglia). Thus, embodiments of the present disclosure may direct therapies or treatments capable of damaging nerves of the lung sufficient to reduce an ability of those nerves to send nerve signals. For example, afferent receptors and nerve fibers may be impaired from sending nerve signals to the CNS 109, while efferent nerve fibers, nerve cell bodies, and nerve trunks may be impaired from sending nerve signals to, e.g., smooth muscle to evoke bronchoconstriction and mucus production, among other responses.

In some embodiments, selective and partial denervation of the bronchial sensory vagal afferent fibers, and in particular the C and RAR fibers having endings in the epithelial layer, may result in more stable or normal vagal afferent activity and nervous system input from the lung.

The interpretation or preprocessing of afferent signals in ganglia may filter the sensory input to the CNS. That is, thresholds may exist for signals to be allowed to pass to the CNS so that many nerves may need to fire within a time period for the signal to be transmitted. Also, secondary effects caused by the initial response can cause a greater intensity and amplification of the response. In some embodiments, reducing afferent input may cause an irritant response that would otherwise reach the threshold for passing to the CNS to fail to be perceived as reaching the threshold. Thus, in some embodiments, reducing afferent input from an area of the lung (e.g., upper airways, central airways, or lower airways) may result in a significant reduction in reflex bronchoconstriction. Thus, in some embodiments, a damaged nerve may require an increased amount of stimulus before sending a nerve signal to the central nervous system, as compared to a pre-damaged state of the nerve.

Nerves can be damaged in the right main bronchus, left main bronchus, or both, as treating only one of the right or left main bronchi may be sufficient for a significant reduction in bronchoconstriction and/or mucus production, as the right and left vagus nerves traverse along the right and left main bronchi, respectively. Additionally, the CNS 109 may interpret signal from only one of the left side or right side of the lung as an anomaly, which may result in a reduced cholinergic reflex, reduced bronchoconstriction, and/or reduced mucus secretion response.

In some embodiments, bronchoconstriction and mucus secretion caused by reflex parasympathetic nerve activity may be reduced. In some embodiments, airway inflammation and remodeling also may be reduced. Sensations of breathlessness (e.g., dyspnea) may be reduced by eliminating some of the afferent activity contributing to the Hering-Breuer reflex, possibly reducing the occurrence of dynamic hyperinflation. By selectively destroying sensory nerves/irritant receptors in the airway, reflex-mediated bronchoconstriction response to various irritant stimuli (e.g., smoke, pollution, etc.) that often trigger acute exacerbations of COPD may be reduced.

The denervation may be superficial to lung airway surfaces and/or may be applied to a depth beyond lung airway surfaces, superficially on the lung airway surfaces, interstitially within the lung airway wall space, and outside the lung airway wall (as some nerve trunks are exterior to the lung airway wall). The target airways may be first to higher generation bronchi (e.g., up to the 10th generation bronchi or beyond). In some embodiments, it may be undesired to treat the trachea in order to preserve the cough reflex. In some embodiments, energy or an agent may be applied to the bronchial branch points (e.g., bifurcations or the like) where RAR fibers are common. Additionally, the concentration of irritants may be relatively high around the bronchial branch points, resulting in a higher nervous system response than other areas of the lung. Denervation may also occur deeper in the airway wall, where both afferent and efferent nerves may be disposed along nerve trunks.

Denervation can be partial, e.g., in many small areas along the airway, as a spiral, in a non-circumferential pattern, in a plurality of spotted treatments, or in another suitable pattern. By treating the airway in this manner, afferent activity may be reduced while allowing for a rapid recovery of the epithelium, and reduced inflammation. Also, the cough response may be reduced but not eliminated, and mucociliary action may be reduced for a short while but not eliminated. This may be advantageous over other denervation procedures that eliminate or substantially impair mucociliary action. This may also reduce the possibility of strictures forming or other adverse events from occurring. In some embodiments, these benefits also may be achieved by only treating the portion(s) of the airway diameter where the highest nerve density and/or nerve trunk is located. These regions may be identified prior to a procedure, or may be determined, by e.g., visual analysis. In one embodiment, optical coherence tomography may be utilized to identify specific treatment regions.

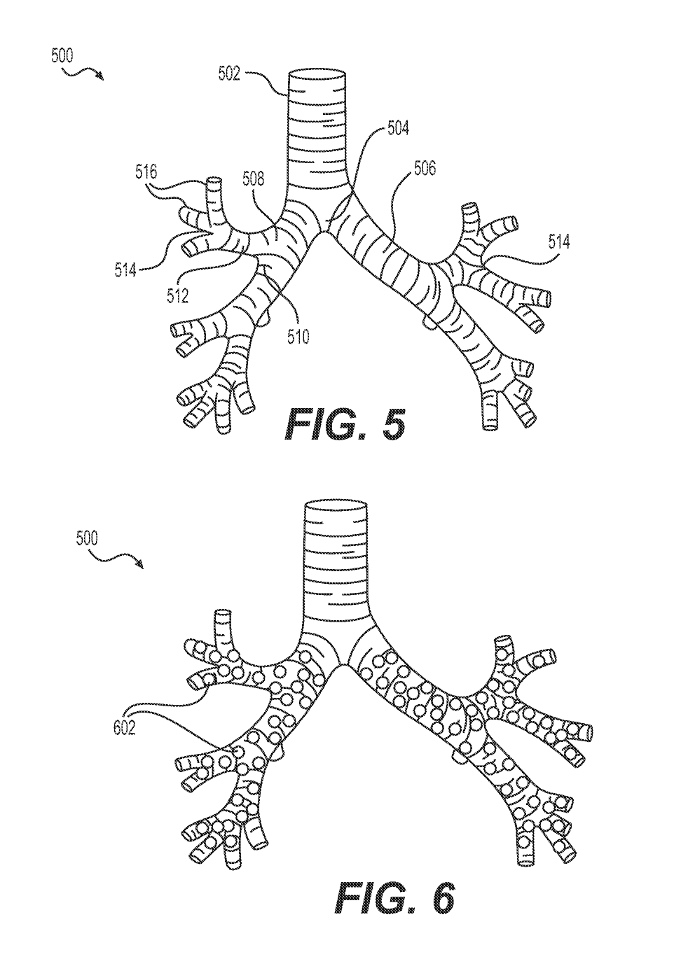

A lung airway system 500 is depicted in FIGS. 5-9. The trachea 502 may extend down from the larynx and convey air to and from the lungs. The trachea 502 may divide into right main bronchus 508 and left main bronchus 506, which in turn form lobar, segmental, and sub-segmental bronchi or bronchial passageways. Right main bronchus 508 and left main bronchus 506 may branch apart at the carina 504 (i.e., a bifurcation at the distal end of trachea 502). Each of right main bronchus 508 and left main bronchus 506 may branch off into subsequent generations of airways. For example, a second generation of airways (e.g., bronchi 512) may extend from a branch point 510. Branch point 510 may be a bifurcation, or may alternatively branch off into any suitable number of subsequent generation bronchi 512. A third generation of airways (e.g., bronchi 516) may extend from a branch point 514 that is located at the distal end of bronchi 512. Lung airway system 500 may include any suitable number of bronchial generations. Eventually, lung airway system 500 may extend to a plurality of terminal bronchioles (not shown). A plurality of alveolar sacs containing alveoli may be disposed at each of the plurality of terminal bronchioles to perform gas exchange during inhalation and exhalation.

In one embodiment, a treatment procedure may be applied to lung airway system 500 in a variety of discrete treatment locations 602 (FIG. 6). Treatment locations 602 may be of any suitable geometry, such as, e.g., circular, square, irregular, lined, or the like. A plurality of treatment locations 602 may be located around the carina 504, left main bronchus 506, right main bronchus 508, branch points 510 and 514, bronchi 512 and 516, and/or subsequent generation branch points and bronchi. In some embodiments, treatments may be concentrated along the carina 504 and/or other branch points, as there may be a concentration of afferent nerve fibers and receptors at these locations. In some embodiments, treatments may be performed at or adjacent to only carina 504 and/or other branch points, due to the concentration of nerve fibers and receptors at these locations.

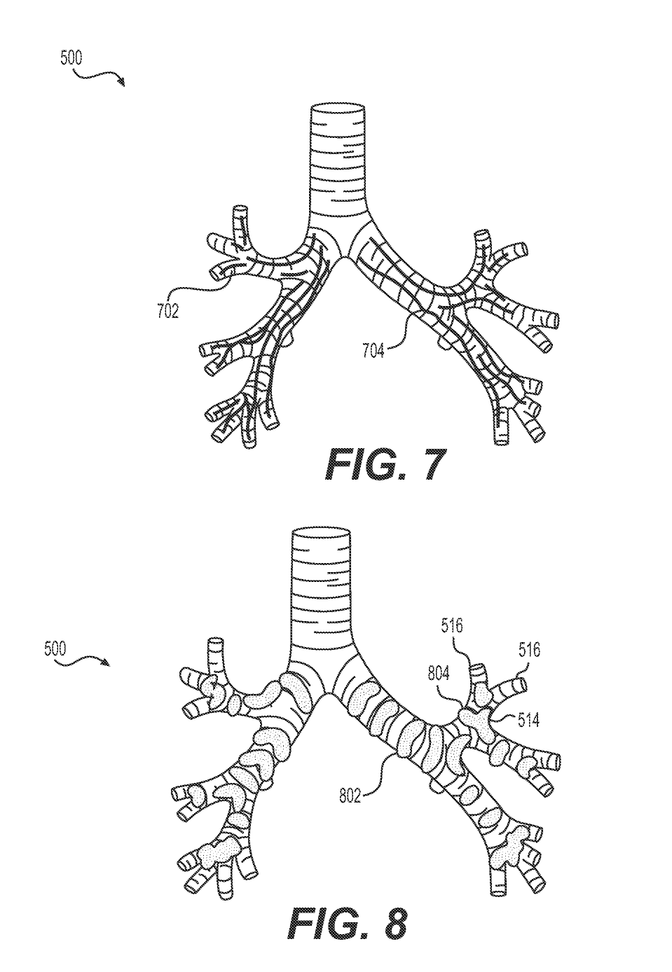

As shown in FIG. 7, one or more treatment locations 702 may be axially-oriented (i.e., arranged along a length of an airway substantially parallel to an axis of the airway), and may extend adjacent branch points 510 and 514, and/or subsequent generation branch points, and/or along the carina 504, left main bronchus 506, right main bronchus 508, bronchi 512 and 516, and/or subsequent generation bronchi. In some embodiments, the one or more axial treatment locations 702 may traverse one or more sections of lung airway system 500. In some embodiments, axial treatment locations 702 may be continuous, or alternatively, near-continuous such that axial treatment locations 702 have one or more breaks disposed along the treatment path. For example, an axial treatment location may extend from carina 504, through left main bronchus 506, branch point 510, bronchus 512, and branch point 514, toward bronchus 516.

As shown in FIG. 8, one or more treatment locations 802 may be disposed circumferentially about the airways of lung system 500, and may incorporate one or more of the carina 504, left main bronchus 506, right main bronchus 508, branch points 510 and 514, bronchi 512 and 516, and/or subsequent generation branch points and bronchi. In one embodiment, a single circumferential treatment location 804 may be disposed within two bronchi 512, and may encompass a branch point 510. Circumferential treatment locations can circumscribe an entirety of a bronchus, or less than an entirety of a bronchus. In one embodiment, a spiral, sinusoidal, or wavy pattern may extend along the airways of lung system 500. Leaving a portion of a circumference of a bronchus untreated may result in certain benefits such as, e.g., less inflammatory reaction, enhanced or faster healing of the epithelial and sub-mucosal layers, staged treatment options, flexibility in therapy aggressiveness plans, and minimization of tissue disruption depending on the severity of symptoms, among others.

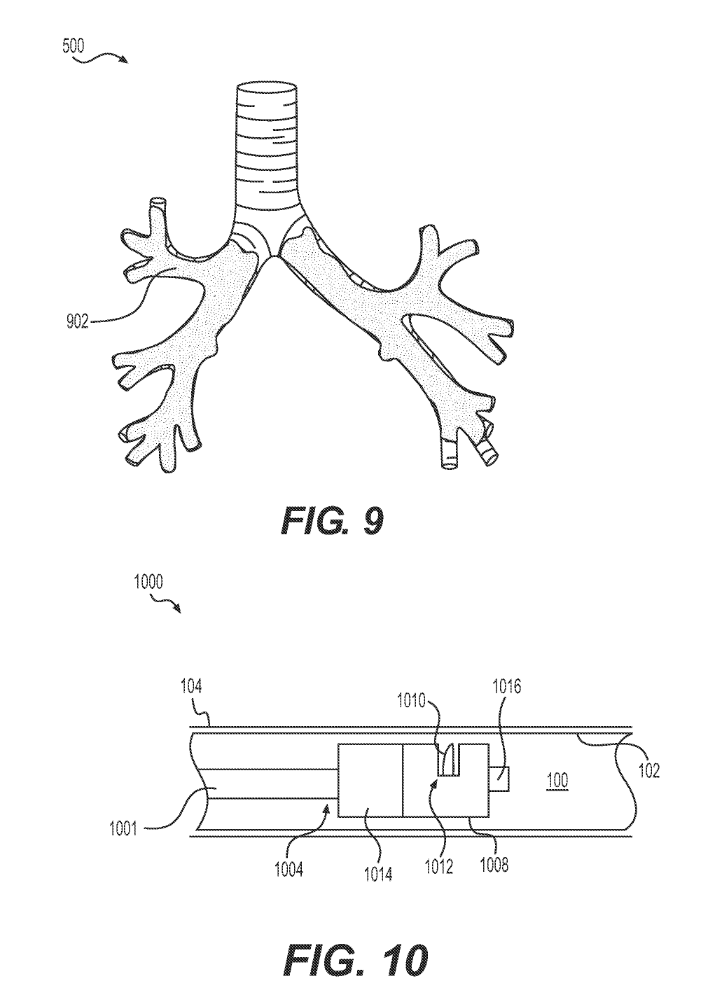

As shown in FIG. 9, one or more treatment locations 902 may include an entirety or a substantial entirety of the left main bronchus 506, right main bronchus 508, branch points 510 and 514, bronchi 512 and 516, and/or subsequent generation branch points and bronchi. Such a treatment pattern may be used, for example, when the patient exhibits severe symptoms of COPD, or other ailments of the lung, and other treatment patterns (such as described with reference to FIGS. 6-8) are shown to be insufficient to combat the severe symptoms. In some embodiments, one or more treatment locations (not shown) may include an entirety or a substantial entirety of carina 504.

When treating carina 504, as it is close to the esophagus, energy or agent delivery may be controlled so as not to damage the esophagus. In some embodiments, treating carina 504 may affect the entire lung and reduce symptoms of lung disease significantly (in some patients, most coughing may be caused by stimulation at the carina 504). In some embodiments, treatments can be tailored based upon the symptoms of the patient. For a patient suffering from COPD, later branch points may be treated (e.g., 2^{nd} generation airways or later) while not treating carina 504, in order to preserve some cough function. If the patient suffers from too much coughing symptoms, the patient may be treated at both carina 504 and later branch points. In some patients suffering from chronic bronchitis, cough symptoms and mucus production may be severe, and thus it may be desirable to treat carina 504. It should be noted that branch points 510 and 514 may be optimal treatment targets, as there may be a relatively high concentration of afferent and efferent fibers in those areas.

In some embodiments of the present disclosure, nerves of the lung airways may be sufficiently damaged to prevent regeneration of the nerves. In other embodiments, nerves may regenerate after being damaged, but may exhibit a reduced sensitivity to stimuli. In some embodiments, if treating a nerve trunk or a longer section of a nerve, a longer length of damaged nerve and/or more areas of treatment may be desirable to reduce the possibility of nerve regeneration. In one embodiment, nerve regeneration may be slowed or stopped by destroying a nerve cell body or by causing fibrosis in the region of the nerve being ablated. The formation of excess fibrous connective tissue in the lungs may prevent the re-innervation of denervated areas. That is, causing a level of fibrosis in the lungs via the treatments of the present disclosure may prevent the regeneration of damaged nerves. In some embodiments, nerves treated by the present disclosure may regenerate at a rate that is slower than a normal rate of regeneration (e.g., at a rate of less than 1 mm growth per day, or another suitable rate).

In some embodiments, treatments may be directed toward efferent post-ganglionic nerve cell bodies that are located in the pulmonary plexuses along the branches of the bronchial tree. In some embodiments, one or more of afferent denervation and efferent denervation may be achieved by inducing fibrosis in a treatment zone to reduce nerve regeneration.

The sympathetic innervation along the bronchi is bronchodilative, and therefore, in some embodiments, it may be undesirable to destroy or otherwise damage sympathetic nerves. However, in those embodiments where sympathetic nerves are treated, destroyed, or otherwise damaged, they may be able to easily regenerate as their nerve cell bodies are located in paravertebral sympathetic ganglia of the sympathetic trunk, which may be located far away from the treatment areas of the present disclosure.

In some embodiments of the present disclosure, symptoms of one or more of the following conditions may additionally or alternatively be reduced: Asthma, Chronic Cough, Chronic Bronchitis, Pulmonary Fibrosis, Cystic Fibrosis, Bronchiectasis, or any other condition where bronchoconstriction, mucus hypersecretion, and cough may be present. Bronchial hyperreactivity may also present in patients with congestive heart failure (CHF) and mitral valve stenosis (MVS). Thus, some embodiments of the present disclosure may treat non-airway conditions.

Additionally or alternatively, the denervation therapy may target the pre-ganglionic parasympathetic nerve that runs along the bronchi, post-ganglionic parasympathetic nerve fibers, and/or the ganglia inside the airway walls. In some embodiments, either or all of these nerve structures may be denervated to effectively achieve reduced ACh release, reduced bronchoconstriction, and reduced mucus production in the treated airways and airways distal to the treated regions. The parasympathetic nerves of the vagus may have both cholinergic contractile and non-adrenergic, non-cholinergic (NANC) relaxant innervation. The neurotransmitter for the NANC nerves may be vasoactive intestinal peptide (VIP) and related peptides. Cholinergic and NANC parasympathetic neurons may be localized in discrete parasympathetic ganglia and may be under distinct pre-ganglionic control, with the NANC neurons found exclusively in the myenteric plexus of the esophagus, whereas cholinergic neurons are often situated in ganglia associated with the adventitia of the airway wall. Thus, in some embodiments, the delivery of energy modalities can affect cholinergic innervation in airway walls with a lesser or negligible effect on NANC relaxant innervation. In some embodiments, this may be achieved by controlling the depth of delivery to target nerves and/or ganglia in the region of the airway wall or just outside the airway wall.

In some embodiments of the present disclosure, one or more energy modalities can be applied to damage nerves, including, thermal (resistive and/or infrared), radio-frequency (RF), irreversible electroporation (IRE), reversible electroporation (RE), microwave, laser, ultrasonic (e.g., HIFU), cryo-ablation, radiation, chemical, neurolytic, mechanical and/or other energy modalities.

Neurolytic Agents

In some embodiments, a neurolytic agent may be delivered in a controlled manner to destroy afferent sensory nerves, e.g., C-fibers, RAR receptors, SAR receptors, and the like. The neurolytic agent may be configured to damage or destroy nerve function, including the ability of a targeted nerve to transmit signals. In some embodiments, the neurolytic agent may be indiscriminate, and may damage all types of nerve fibers that it contacts, regardless of size or function. In other embodiments, the neurolytic agent may be configured to damage a specific type of nerve or receptor. For example, a discriminate neurolytic agent may damage only afferent nerves, only a specific afferent nerve, only efferent nerves, or only a specific efferent nerve.

Examples of suitable neurolytic agents include, but are not limited to ethanol, phenol, glycerol, ammonium salt compounds, hypertonic and/or hypotonic solutions, and botulinum toxin (BOTOX). In some embodiments, BOTOX may disrupt the neuromuscular junction by inhibiting vesicular release of neurotransmitter.

In some embodiments, high doses of capsaicin may be delivered to damage afferent C-fibers. Capsaicin may be configured to damage only afferent C-fibers within the epithelial tissue. That is, capsaicin may have a therapeutically negligible effect on efferent fibers and may selectively target afferent nerves. Capsaicin may be delivered in solution at a concentration of 0.01% to 1% weight per volume, or at another suitable concentration. In some embodiments, capsaicin may be delivered in dilute (50%) ethanol, or in another suitable delivery mechanism. The capsaicin may be delivered as solid particles dispersed in a gel or liquid, if desired.

The neurolytic agent may be delivered as a fluid, hydrogel, thermoresponsive gel, or in another suitable manner. In some embodiments, therapy could be delivered such that only the afferent sensory nerves in a local region of neurolytic agent delivery are affected (only in the first through third generation bronchi, for example), or could be delivered such that the transmission of sensory nerve signals from distal airways towards the CNS is impeded. In the embodiment where only a local region of afferent sensory nerves are targeted, the neurolytic agent could be delivered at a shallower depth in the epithelium, such as, e.g., shallower than about 0.1 mm. In the embodiment where transmission of sensory nerve signals from distal airways towards the CNS are impeded, the neurolytic agent may be delivered at a deeper depth, such as, e.g., up to about 5.0 mm, and may be delivered towards a nerve trunk running along the airway.

In some embodiments, the neurolytic agent may be combined with other suitable agents. In one embodiment, the neurolytic agent could be combined with DMSO, or another suitable material to improve diffusion and/or transport of the neurolytic agent to targeted nerves. In another aspect, the neurolytic agent could be combined with a gel such as, e.g., a thermoresponsive gel, to temporarily maintain the neurolytic agent in a desired location. For example, the combined neurolytic agent/gel mixture may be configured to either hold the neurolytic agent on the surface of the airway, within the airway wall, or just outside the airway wall near the nerve trunk. In another embodiment, the neurolytic agent could be combined with a fluoroscopic contrast agent or dye to improve visibility of locations where the neurolytic agent has been applied during a given therapy.

The various neurolytic agents may damage nerve fibers by any suitable mechanism. In one embodiment, phenol may be delivered to damage nerve receptors and fibers. Phenol may damage nerves by inducing protein precipitation, loss of cellular fatty elements, separation of the myelin sheath from the axon, and axonal edema. In another embodiment, ethanol may be delivered to extract phospholipids, cholesterol, and cerebroside from neural tissues, and precipitate mucoproteins and lipoproteins. In some embodiments, and particularly in low doses, ethanol may produce neuritis and nerve degeneration (neurolysis).

Delivery of the neurolytic agent may be through the right main bronchus, left main bronchus, or both, as treating only one of the right or left main bronchi may be sufficient for a significant reduction in bronchoconstriction and/or mucus production, as the right and left vagus nerves traverse along the right and left main bronchi, respectively. In some embodiments, only portions of the vagus nerve that innervate the lungs may be treated. As the main trunk of the vagus nerves additionally may innervate the intestines and other thoracic organs, it may be desirable to only treat the branches of the vagus nerves that innervate the lungs, so as to minimize any residual effects on other organs.

Because the diffusion of a neurolytic agent and its effects may be active over a wide region, exact placement of the neurolytic agent may not be necessary. However, linking the delivery or injection of neurolytic agent with a detection system such as, e.g., an electrode mapping catheter, Doppler ultrasound, and optical coherence tomography, among others, to a localized treatment location may allow for a more specific treatment to be applied (e.g., a single needle design as opposed to a multiple needle design), potentially reducing the neurolytic agent load on adjacent tissues. Further, the various detection systems may identify visual landmarks that may be indicative of nerve locations. For example, in some embodiments, blood vessels may act as a visual landmark of nerve fibers for a physician applying a neurolytic agent. The physician may identify a particular blood vessel and deliver neurolytic agent to the blood vessel or to the tissues surrounding the blood vessel. In one embodiment, an ultrasound sensor may be utilized to ensure an appropriate depth of penetration of a delivery device, e.g., a needle. The ultrasound sensor may be utilized in a similar manner to those used for EBUS in lymph node sampling and biopsies. The ultrasound sensor may help provide assurance that the needle is within or just outside the airway wall, and not penetrating into other nearby structures, such as, e.g., the esophagus.

In some embodiments, after neurolytic agent is applied to damage lung airway nerves, a rinse may be applied to remove residual neurolytic agent from the lung airway. In some embodiments, the rinsing mixture may include one or more of saline, water, ethanol, and Tween. Delivery of the rinsing mixture may be achieved by any suitable method, such as, e.g., spray or injection. The rinsing mixture may be vacuumed, aspirated, or removed from the lung airway in another suitable manner. In some embodiments, the same device may be used to apply the neurolytic agent and then perform the rinse.

Delivery Devices

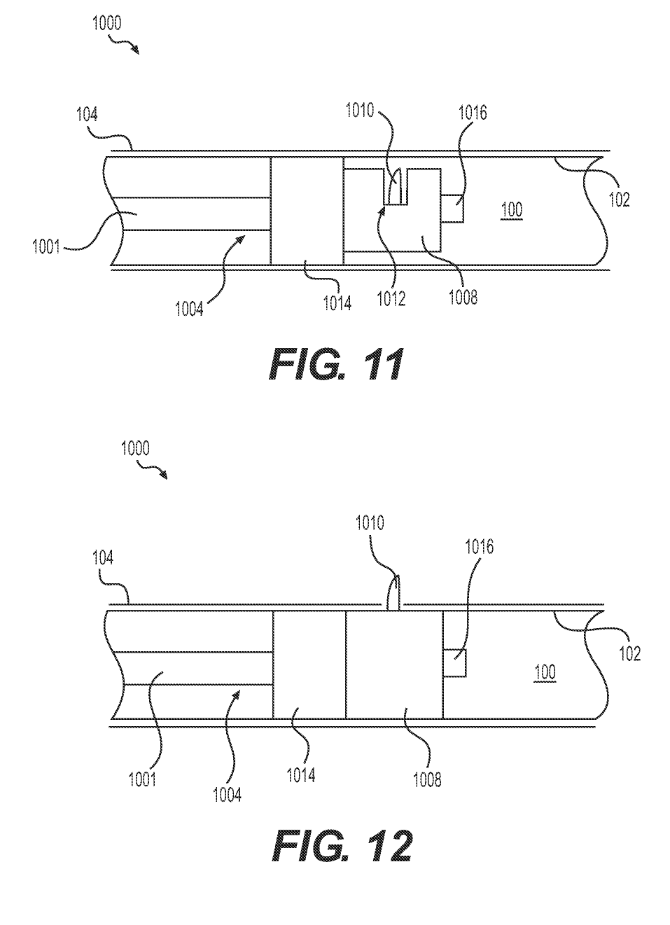

As shown in FIGS. 10-12, a medical device 1000 may include an elongate member 1001 extending from a proximal end (not shown) toward a distal end 1004. An expandable or inflatable member 1008 may be disposed at distal end 1004 and may be configured to be reciprocally movable between an expanded/inflated configuration (referring to FIG. 12), a partially-inflated, partially-deflated configuration (referring to FIG. 11), and an unexpanded/deflated configuration (referring to FIG. 10). Medical device 1000 may include a fluid delivery device 1010 configured to transmit a fluid (e.g., a neurolytic agent) through airway wall 102 and epithelial tissue 104. Fluid delivery device 1010 may be a syringe, needle, or other suitable fluid delivery device capable of delivering fluid through airway wall 102. In some embodiments, a distal end of fluid delivery device 1010 may be beveled or otherwise sufficiently sharp to pierce through tissue. Fluid delivery device 1010 may be fluidly coupled to a source of neurolytic agent (not shown). Fluid delivery device 1010 may be disposed in a recess 1012 located in an outer circumference of inflatable member 1008.

Medical device 1000 may also include a guidance/anchoring mechanism 1014 configured to direct medical device 1000 via, e.g., direct visualization, ultrasound (e.g., EBUS), CT-guidance, Optical Coherance Tomography (OCT), or electrical characterization of nerve location (e.g., by sensing electrical nerve or muscle signals). Guidance mechanism 1014 may be configured to be reciprocally movable between an unexpanded/deflated configuration (referring to FIG. 10) and an expanded/inflated configuration (referring to FIGS. 11 and 12). Guidance mechanism 1014 may be inflated by any suitable mechanism. For example, guidance mechanism 1014 may be inflated by an infusion of saline, air, or other gases or liquids from a lumen of elongate member 1001. Alternatively, guidance mechanism 1014 may include another suitable expandable member, such as, e.g., a stent or a basket. When guidance mechanism 1014 is inflated, it may act as an anchor for medical device 1000 within airway 100. Further, when guidance mechanism 1014 is inflated, and inflatable member 1008 is deflated, medical device 1000 may be in a partially-inflated, partially-deflated configuration (shown only in FIG. 11). Guidance mechanism 1014 may also include a detector for detecting nerves of the lung. Alternatively or additionally, all or a portion of medical device 1000 may be formed of a radiopaque material so that it can be visualized under fluoroscopic guidance, or may otherwise include radiopaque markers, visual indicators viewable by a bronchoscopic camera, or other imaging markers for guidance. The markers may be used to ensure that a correct direction of therapy is applied. In some embodiments, guidance mechanism 1014 and/or inflatable member 1008 may be prevented from activating until the marker is appropriately positioned. It should be noted that any medical device, delivery device, or elongate member delivering any suitable agent or energy modality may include a guidance mechanism 1014.

As shown in FIGS. 10 and 11, fluid delivery device 1010 may be recessed within an outer periphery or circumference of inflatable member 1008. Inflatable member 1008 may be inflated to move medical device 1000 from the partially-inflated, partially-deflated configuration of FIG. 11 to the inflated configuration of FIG. 12. Inflatable member 1008 may be inflated by a substantially similar mechanism as described above with reference to guidance mechanism 1014. As shown in FIG. 12, the inflation of inflatable member 1008 may cause fluid delivery device 1010 to pierce airway wall 102 and/or epithelial tissue 104 so that a fluid may be delivered to nerves of the lung. Thus, in the inflated configuration of FIG. 12, fluid delivery device 1010 may extend from the outer periphery or circumference of inflatable member 1008. An amount of extension of fluid delivery device 1010 may dictate a controlled depth of penetration for fluid delivery device 1010, and therefore may also affect neurolytic agent delivery. For example, fluid delivery device 1010 may be configured to penetrate a shallow depth (e.g., about 0.5 mm) to target nerves disposed within the epithelial tissue. In another embodiment, fluid delivery device 1010 may be configured to penetrate further (e.g., about 2.0 mm to) to target nerves disposed along a nerve trunk or within the smooth muscle layer. In one embodiment, the penetration depth of fluid delivery device 1010 may be controlled by an inflation pressure. For example, a greater pressure placed on inflatable member 1008 may penetrate fluid delivery device 1010 further into lung tissue. In one embodiment, the portion of inflatable member 1008 under a given fluid delivery device 1010 may be designed to expand more than other regions of inflatable member 1008 (e.g., this portion of the inflatable member 1008 could exhibit greater compliance than other regions of inflatable member 1008). Thus, the inflation pressure of inflatable member 1008 may be an indicator for depth of penetration. Alternatively, a depth-of-penetration visual marker may be disposed on one or more of inflatable member 1008 and fluid delivery device 1010, and may be viewable through, e.g., a bronchoscope or another visualization device (e.g., fluoroscopy, in the case of a radiopaque marker).

Alternatively, medical device 1000 may include a plurality of fluid delivery devices disposed within the outer periphery or circumference of inflatable member 1008. In one alternative embodiment, medical device 1000 may include at least two fluid delivery devices 1010 disposed at approximately an axial midpoint of inflatable member 1008, and separated by approximately 180°. In other embodiments, fluid delivery devices 1010 may be arranged in columns, rows, or other suitable arrangements. In one embodiment, the plurality of fluid delivery devices 1010 may be in the same axial plane, or may be offset from one another so as to create a spiral-shaped delivery of neurolytic agent. In some embodiments, medical device 1000 may not include a guidance/anchoring mechanism 1014.

In one alternative embodiment, neurolytic agent may be delivered via other suitable mechanisms, such as, e.g., any suitable syringe or needle. In yet another alternative embodiment, delivery of the neurolytic agent may be achieved via a syringe or needles penetrating the wall of the esophagus to target the nerve(s) in the airways.

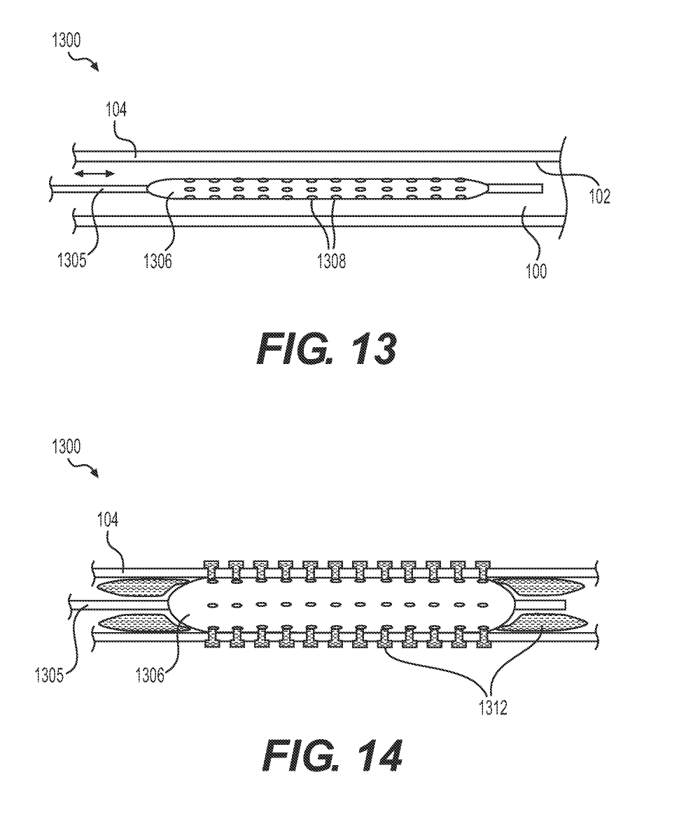

As shown in FIG. 13, a medical device 1300 may include an inflatable member 1306 (such as, e.g., a balloon) having a plurality of pores 1308 disposed along a circumference of inflatable member 1306. Inflatable member 1306 may be coupled to and/or extend distally from an elongate member 1305, such as, e.g., a sheath, catheter, or the like. Elongate member 1305 and inflatable member 1306 may extend distally from the distal end of an bronchoscopic member (not shown), such as, e.g., a bronchoscope or the like. Inflatable member 1306 may be fluidly coupled to a fluid source via a lumen of elongate member 1305.

Medical device 1300 is shown in FIG. 13 in a deflated configuration, while, in FIG. 14, medical device 1300 is shown in an inflated configuration. To move from the deflated configuration of FIG. 13 to the inflated configuration of FIG. 14, a fluid, such as, e.g., a neurolytic agent 1312 may be delivered from the fluid source to inflatable member 1306. In the inflated configuration, an outer surface of inflatable member 1306 may contact the surface of airway wall 102. Further, in the inflated configuration, neurolytic agent 1312 may flow through pores 1308. As neurolytic agent 1312 exits pores 1308, it may flow through airway wall 102 and epithelial tissue 104 toward nerves of the lung. Medical device 1300 may include a plurality of pores along the length of inflatable member 1306 arranged in columns and rows, although other suitable configurations are also contemplated. Pores 1308 may be formed with any suitable diameter ranging from e.g., microns to millimeters. In one embodiment, medical device 1300 may include several hundred pores 1308 having a 0.5 micron diameter. In some embodiments, pores 1308 may be oriented in a pattern such as, e.g., a spiral pattern to avoid some adverse effects if the neurolytic agent is capable of causing long-term damage, such as, e.g., damage to cilia. Alternatively, pores 1308 may be arranged in another suitable pattern, for example, to achieve the treatment patterns shown by FIGS. 6-9 of the present disclosure.

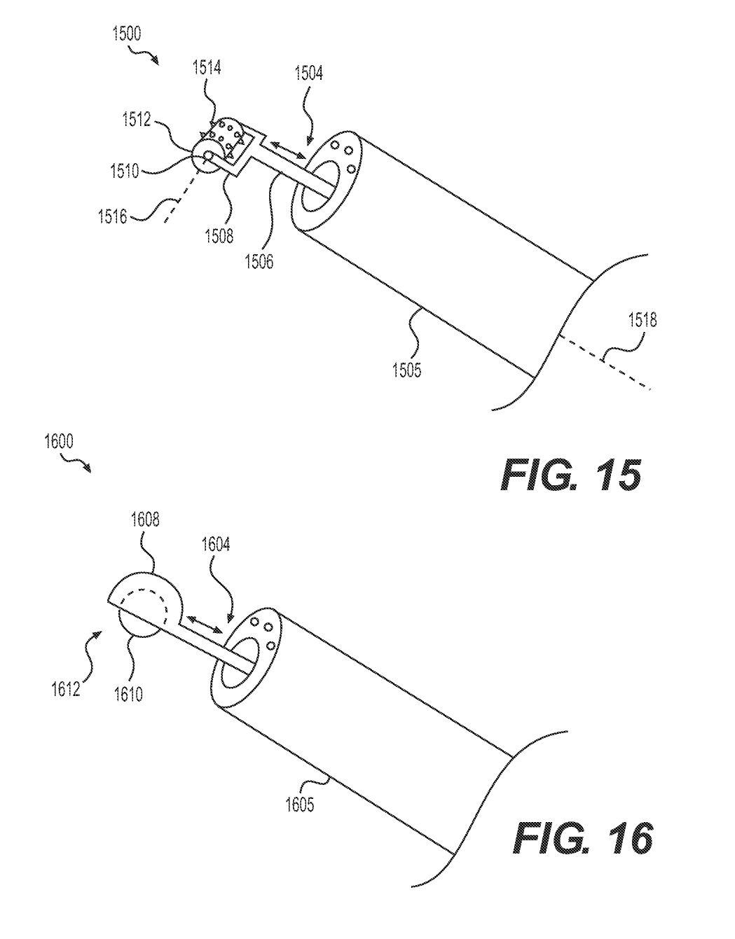

As shown in FIG. 15, a medical device 1500 may include a bronchoscopic member 1505 extending from a proximal end (not shown) toward a distal end 1504. An elongate member 1506, such as, e.g., a sheath, catheter, or the like, may extend distally from distal end 1504. A support 1508 may be disposed at the distal end of elongate member 1506. A deployment member 1512 may be disposed within support 1508. Support 1508 may be generally U-shaped with a base and two legs, for example. However, other suitable configurations of support 1508 are also contemplated, including but not limited to C-shapes, V-shapes, and the like. Deployment member 1512 may be cylindrical or have another suitable shape such that deployment member 1512 rolls as medical device 1500 is moved along a longitudinal axis 1518. Deployment member 1512 may be movably supported by ends 1510 of support 1508 via suitable fastening mechanisms such as, e.g., hinges, rollers, pins, or the like. As medical device 1500 is moved longitudinally, deployment member 1512 may roll about a rolling axis 1516 that is offset from, e.g., substantially perpendicular to, longitudinal axis 1518 of medical device 1500. A plurality of fluid delivery devices 1514, such as, e.g., needles, may be disposed along the circumference of deployment member 1512 to deliver a neurolytic agent through a lung airway wall and epithelial tissue toward nerves of the lung. Thus, in operation, as medical device 1500 is advanced longitudinally along longitudinal axis 1518 and against an airway wall, deployment member 1512 may roll simultaneously about rolling axis 1516, causing fluid delivery devices 1514 to penetrate airway wall 102 at different locations to deliver fluid toward one or more nerves of the lung. In some embodiments, fluid delivery devices 1514 and deployment member 1512 may be fluidly coupled to a source of neurolytic agent (not shown) that flows through a lumen of elongate member 1506 and support 1508.

Fluid delivery devices 1514 may be in any suitable configuration and pattern, such as, e.g., rows, columns, random arrangements, aligned with each other, and/or have variable lengths to control depths of penetration and neurolytic agent delivery. Fluid delivery devices 1514 may be configured so that they deliver agent only upon insertion into tissue. For examples, tissue penetration may open the tip of fluid delivery devices 1514, and may close upon removal from the tissue (e.g., a biased or valved tip design). This may be beneficial if it is desired to only deliver agent within the tissue, and not superficially along the airway. Alternatively, fluid delivery devices 1514 may be open at all times to deliver neurolytic agent within airway lumens when fluid delivery devices 1514 are not within the tissue.

As shown in FIG. 16, a medical device 1600 may include a bronchoscopic member 1605 extending from a proximal end (not shown) toward a distal end 1604. An elongate member 1606, such as, e.g., a sheath, catheter, or the like, may extend distally from distal end 1604. A support 1608 may be disposed at the distal end of elongate member 1606. A deployment member 1610 may be disposed within a cavity 1612 of support 1608. Deployment member 1610 may be spherical or have another suitable shape such that deployment member 1610 rotates within cavity 1612 as medical device 1600 is moved in any direction. Deployment member 1610 may be supported within cavity 1612 of support 1618 by any suitable means, including friction fits or the like. In one embodiment, a side hole of cavity 1612 may have a smaller diameter than a diameter of deployment member 1610 such that deployment member 1610 can roll freely within cavity 1612.

In some embodiments, deployment member 1610 and support 1608 may be fluidly coupled to a source of neurolytic agent (not shown) via elongate member 1606. In an alternative embodiment, deployment member 1610 may be coated with a neurolytic agent. Thus, in operation, as medical device 1600 is advanced along any direction, deployment member 1610 may rotate simultaneously in the substantially same direction as medical device 1600 to deliver a neurolytic agent to a treatment location to one or more nerves of the lung via airway wall 102. In one embodiment, deployment member 1610 may be coupled to a lumen (not shown) configured to deliver neurolytic agent to deployment member 1610 via pressure or another mechanism. In another embodiment, neurolytic agent may be delivered to deployment member 1610 by a length of porous fibers that are fluidly coupled to a source of neurolytic agent. In some embodiments, neurolytic agent may be transferred from a neurolytic agent source to deployment member 1610 via capillary action along the length of porous fibers. In some embodiments, deployment member 1610 may include one or more fluid delivery devices, such as, e.g., one or more needles.

Alternatively, deployment member 1610 and cavity 1612 may act as a ball valve. That is, when no pressure is applied by to deployment member 1610 by tissue, then deployment member 1610 may seals to the side opening in support 1608. When deployment member 1610 presses against tissue, deployment member 1610 may retreat within the opening of support 1608, revealing portions of the opening to permit agent to flow out of cavity 1612.

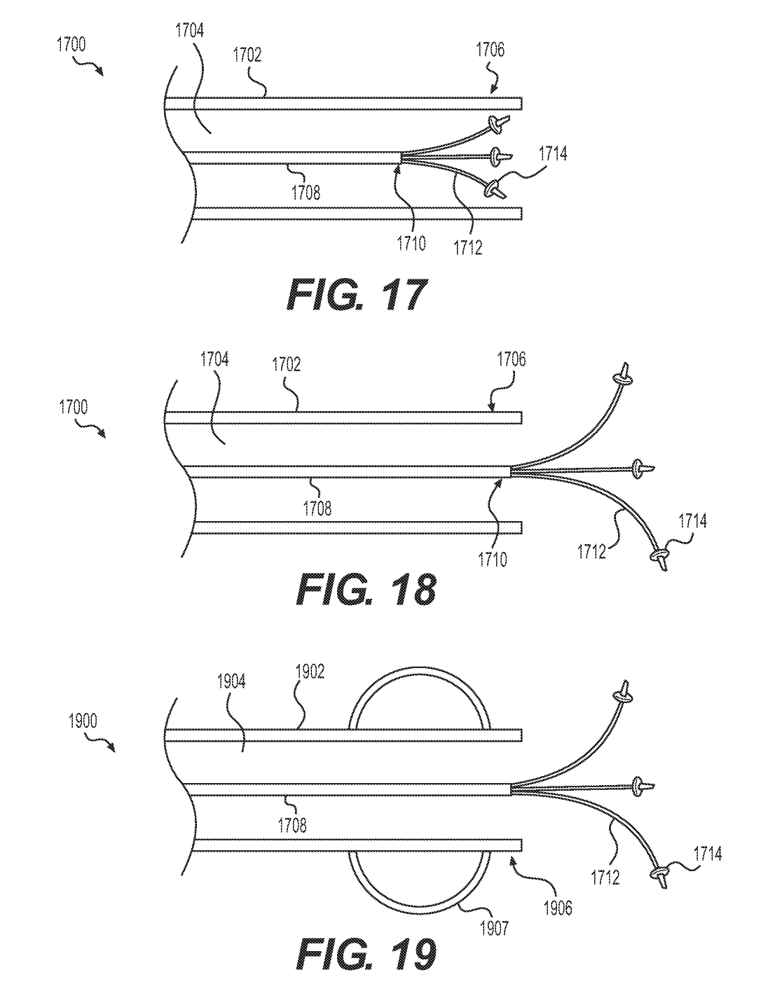

A medical device 1700 configured to deliver a neurolytic agent is depicted in FIGS. 17 and 18. Medical device 1700 may include an elongate member 1702 having a lumen 1704 that extends from a proximal end (not shown) toward a distal end 1706 of elongate member 1702. Elongate member 1702 may be a bronchoscope, or may alternatively be a catheter or other suitable member that extends distally from the distal end of a bronchoscope. A movable member 1708 may be disposed within lumen 1704 and may be configured to be reciprocally movable between a first position shown in FIG. 17, and a second position shown in FIG. 18. Movable member 1708 may be a hollow tube (e.g., a hypotube) or other suitable mechanism capable of reciprocal movement. Examples of suitable materials for movable member 1708 may include, but are not limited to, nitinol, other shape memory alloys, stainless steel, polyurethane, ETFE, PTFE, PEBAX, PET, or another suitable polymer or material, or the like.

A plurality of fluid delivery devices 1712 may extend from a distal end 1710 of movable member 1708. In some embodiments, the distal ends of fluid delivery devices 1712 may be beveled or otherwise sufficiently sharp so that fluid delivery devices 1712 can pierce through tissue. Thus, each fluid delivery device 1712 may be a needle or other suitable object configured to deliver or withdraw fluids or other substances. Fluid delivery devices 1712 may be formed of nitinol, stainless steel or other metals, alloys, polymers, or other suitable materials. Fluid delivery devices 1712 may be coupled to a source of neurolytic agent (not shown) through one or more lumens disposed through or along movable member 1708.

As shown in FIG. 17, fluid delivery devices 1712 may be in a substantially retracted configuration. That is, in the retracted configuration, fluid delivery devices 1712 may be constrained by elongate member 1702. Movable member 1708 may be displaced distally by a handle or other suitable actuating mechanism (not shown) to move medical device 1700 from the retracted configuration of FIG. 17 to an expanded configuration shown in FIG. 18. In the expanded configuration, fluid delivery devices 1712 may self-expand to extend radially outward from the longitudinal axis of medical device 1700 to contact an airway wall (not shown). In some embodiments, fluid delivery devices 1712 may be pre-bent into the expanded configuration shown in FIG. 18. Movable member 1708 may be reciprocally movable so that medical device 1700 may deploy neurolytic agent to multiple points along a lung airway, or to multiple lung airways.

A distal stop 1714 may be disposed on the distal end of each fluid delivery device 1712. Distal stops 1714 may be disposed a predetermined distance from the distal end of fluid delivery devices 1712 to control the depth that fluid delivery devices 1712 penetrate a lung airway wall and epithelial tissue. Distal stops 1714 may be formed from the same material as fluid delivery devices 1712, or may be formed of another suitable material. In some embodiments, distal stops 1714 may have an atraumatic configuration so as not to damage the lung airway wall upon contact. Any fluid device described in the present disclosure may additionally include one or more distal stops at a respective distal end.

A medical device 1900 configured to deliver a neurolytic agent is depicted in FIG. 19. Medical device 1900 may include an elongate member 1902 having a lumen 1904 that extends from a proximal end (not shown) toward a distal end 1906 of elongate member 1902. Elongate member 1902 may be substantially similar to elongate member 1702 described with reference to FIGS. 17 and 18, but may additionally include an expandable member 1907 that is disposed at distal end 1906 of elongate member 1902. Expandable member 1907 may be any suitable expandable member such as, e.g., a balloon, basket, mesh, or similar member configured to anchor elongate member 1902 to a target and/or treatment location along an airway wall. Expandable member 1907 may be additionally configured to position elongate member 1902 at another suitable location, e.g., in the middle of a lung airway to allow one or more fluid delivery devices to penetrate the airway wall. Medical device 1900 may include a movable member 1708, fluid delivery devices 1712, and stops 1714 that are substantially similar to those described with reference to FIGS. 17 and 18. An expandable member 1907 may be included on any other embodiment described in the disclosure.

Distal stops 1714 may be disposed at different or the same distances from the distal ends of fluid delivery devices 1712 to achieve different or the same depths of delivery. In the embodiments shown in FIGS. 17-19, three fluid delivery devices are shown, but medical devices 1700 and 1900 may have any other suitable number of fluid delivery devices 1712. In one embodiment, fluid delivery devices 1712 may be arranged at equal spacing from one another, or at another suitable spacing arrangement. In one embodiment, fluid delivery devices may be clustered to one side of the airway so that delivery is restricted to one portion of the airway wall. Fluid delivery devices 1712 may be of differing lengths to treat different axial points along the airway, or may be of the same length to treat the same axial location but different radial portions about a circumference of the airway wall.

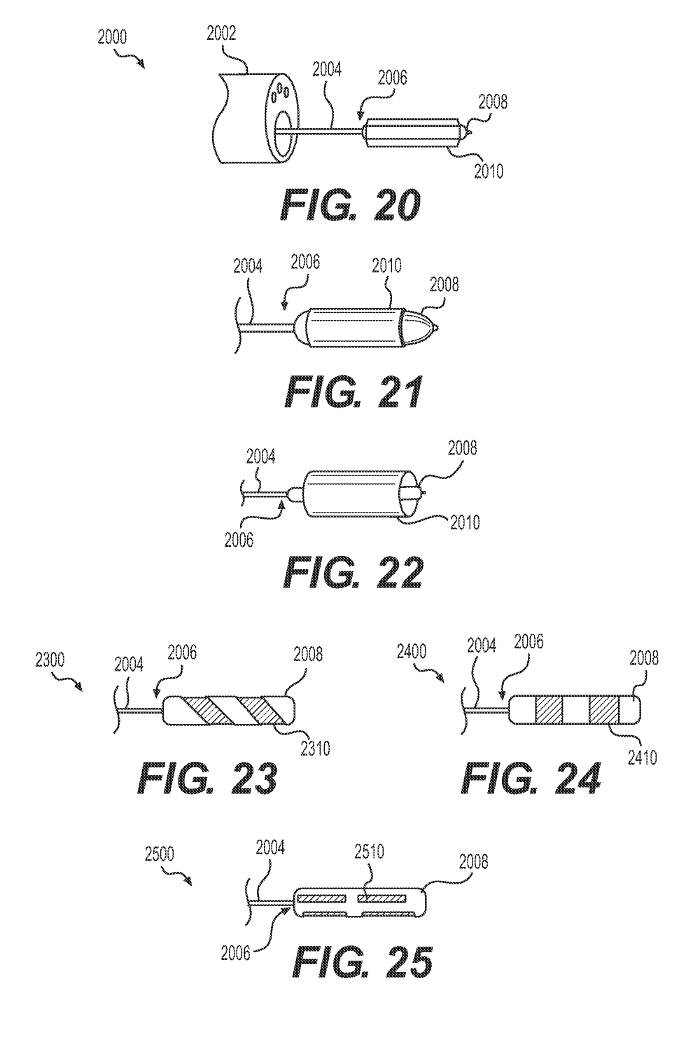

A medical device 2000 is shown in FIGS. 20-22. Medical device 2000 may be configured to deploy one or more strips 2010 containing a neurolytic agent to an airway of the lung. Medical device 2000 may include a first elongate member 2002, such as, e.g., a bronchoscope. A second elongate member 2004 may extend from the distal end of first elongate member 2002. An expandable member 2008 (e.g., an expandable/inflatable balloon) may be coupled to the distal end of second elongate member 2004, and may be movable between a first, unexpanded/deflated configuration (shown in FIGS. 20 and 22), and a second, expanded/inflated configuration (shown in FIG. 21).

Expandable member 2008 may be expanded until strip 2010 is placed into contact with an inner surface of a lung airway wall. In some embodiments, the strips 2010 may immediately dissolve once strips 2010 make contact with the inner surface of a lung airway wall, passing neurolytic agent through the airway wall and epithelial layer to reach, e.g., nerves of the lung. In other embodiments, strips 2010 may be configured to dissolve over a period of time, e.g., seconds, minutes, hours, days, weeks, months, or another suitable period of time. In such configurations, strips 2010 may include one or more adhesives to allow strips 2010 to adhere to the inner surface of a lung airway wall. Strips 2010 may be formed at least partially from a hydrophobic or another suitable polymer. Strips 2010 may additionally or alternatively include other suitable elements such as, e.g., strip-forming polymers, plasticizers, stabilizing agents, and thickening agents, among others.

Once strips 2010 have dissolved within or along the lung airway, or have otherwise been deployed (FIG. 21), second elongate member 2004 may be moved back to the first, retracted configuration (FIG. 22), and second elongate member 2004 and expandable member 2008 may be removed from the airway. In some embodiments, varying strips along the same expandable member 2008 may include different agents for treating different symptoms.

Strips 2010 may be disposed in any suitable shape along expandable member 2008. A medical device 2300 is depicted in FIG. 23 that may be substantially similar to medical device 2000, except that medical device 2300 may include one or more helical strips 2310 disposed on expandable member 2008. A medical device 2400 is depicted in FIG. 24 that may be substantially similar to medical device 2000, except that medical device 2400 may include one or more circumferential strips 2410 disposed on expandable member 2008. Circumferential strips 2410 may extend along an entire circumference of expandable member 2008, or may alternatively only extend partway along the circumference of expandable member 2008. A medical device 2500 is depicted in FIG. 25 that may be substantially similar to medical device 2000, except that medical device 2500 may include one or more axial strips 2510 disposed on expandable member 2008. That is, axial strips 2510 may have a length that extends along a longitudinal axis of medical device 2500. Medical device 2500 may include a plurality of columns of strips 2510 disposed along expandable member 2008. In some embodiments, the columns of strips 2510 may be longitudinally staggered from one another. In other embodiments, each strip 2510 of a respective column may be disposed at the same longitudinal position as a corresponding strip 2510 from another column.

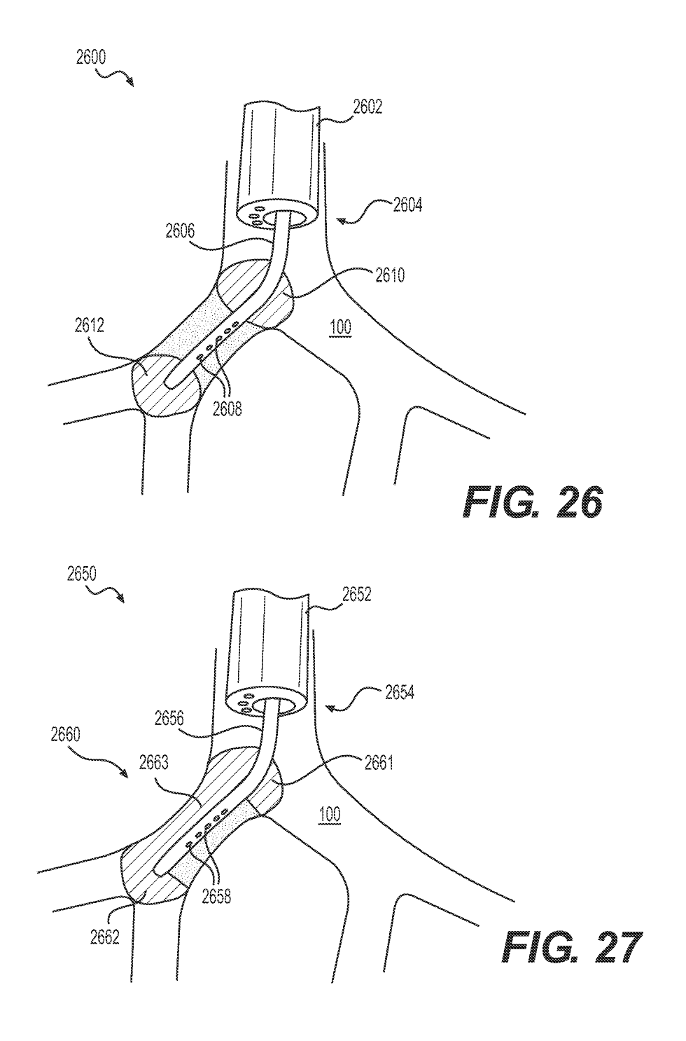

A medical device 2600 is shown in FIG. 26. Medical device 2600 may include a first elongate member 2602, such as, e.g., a bronchoscope. A second elongate member 2606 may extend from a distal end 2604 of first elongate member 2602. Medical device 2600 may include one or more openings 2608 disposed along second elongate member 2606. Each opening 2608 may be coupled to a source of neurolytic agent (not shown), and may be configured to deliver an amount of neurolytic agent to lung airway 100. Each opening 2608 may be configured to deliver neurolytic agent in any suitable manner, such as, e.g., as a sprays, jets, trickles, streams, or the like. Further, a given opening 2608 may be configured to deliver neurolytic agent along or offset to a longitudinal axis of second elongate member 2606. In some embodiments, a given opening may be configured to deliver neurolytic agent in a proximal direction, a distal direction, or in a direction that is substantially orthogonal to the longitudinal axis of second elongate member 2606. In some embodiments, openings 2608 may be configured to deliver different agents (e.g., via different lumens within second elongate member 2606), so that a user can selectively deliver agents to desired treatment sites.

A proximal expandable member 2610 may be disposed proximal to openings 2608 along second elongate member 2606. A distal expandable member 2612 may be disposed distal to openings 2608 along second elongate member 2606. Each of proximal expandable member 2610 and distal expandable member 2612 may be movable between a first, deflated configuration (not shown), and a second, inflated configuration (shown in FIG. 26). Expandable members 2610 and 2612 may be a balloon, or similar, expandable member configured to create a seal around a treatment region to allow an operator to achieve specificity of treatment along a portion of airway 100 for delivering neurolytic agent(s). In an alternative embodiment, expandable members 2610 and 2612 may not be configured to form a seal around a treatment region, and may be formed as an expandable basket, or as a another suitable expandable member.

In the region sealed between expandable members 2610 and 2612, neurolytic agent and rinsing agent may be injected and aspirated simultaneously to create a continuous circulation of neurolytic agent with efficient rinsing.

In an alternative embodiment, medical device 2600 may include only one of proximal expandable member 2610 and distal expandable member 2612. In yet another alternative embodiment, medical device 2600 may include additional expandable members and openings along second elongate 2606 such that a plurality of treatment regions can be formed in airway 100 by medical device 2600. For example, a medical device may include a first, second, and third expandable member. A first set of openings may be disposed between the first and second expandable members, and a second set of openings may be disposed between the second and third expandable members.

A medical device 2650 is shown in FIG. 27. Medical device 2650 may include a first elongate member 2652, such as, e.g., a bronchoscope. A second elongate member 2656 may extend from a distal end 2654 of first elongate member 2652. Medical device 2650 may include one or more openings 2658 disposed along second elongate member 2656 and are substantially similar to openings 2608 described with reference to FIG. 26. Each opening 2658 may be coupled to a source of neurolytic agent (not shown), and may be configured to deliver an amount of neurolytic agent to lung airway 100.

An expandable member 2660 may be disposed proximally, distally, and partially circumferentially around openings 2608 along second elongate member 2606. That is, expandable member 2660 may have a proximal portion 2661, a distal portion 2662, and a circumferential portion 2663 disposed around openings 2608. Expandable member 2660 may be movable between a first, unexpanded/deflated configuration (not shown), and a second, expanded/inflated configuration (shown in FIG. 27). Expandable member 2660 may be a balloon, or similar, expandable member configured to create a seal around a partially-circumferential treatment region to allow an operator to achieve specificity of treatment along a portion of airway 100 for delivering neurolytic agent(s). That is, expandable member 2660 may be configured to deliver neurolytic agent to an isolated, radial portion of airway 100.

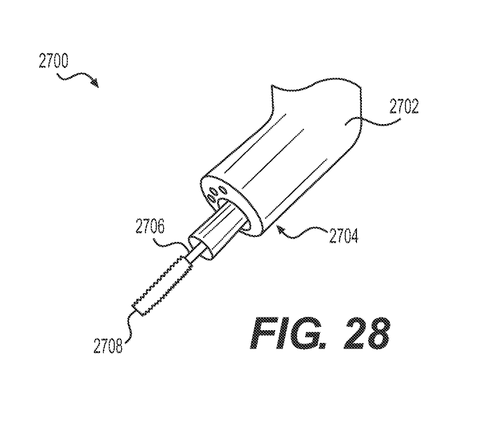

A medical device 2700 is shown in FIG. 28. Medical device 2700 may include a first elongate member 2702, such as, e.g., a bronchoscope. A second elongate member 2706 may extend from a distal end 2704 of first elongate member 2702. The proximal end of second elongate member 2706 may be coupled to a source of neurolytic agent (not shown). An applicator 2708 may be structurally and fluidly coupled to the distal end of second elongate member 2706. Applicator 2708 may be formed from a porous, pressed bundle of fibers, or be a brush. In some embodiments, the pressed bundle of fibers may include felt or another suitable porous fiber. In some embodiments, second elongate member may be a hollow lumen that delivers neurolytic agent to applicator 2708 by pressure or another suitable mechanism. In an alternative embodiment, second elongate member 2706 may be made of the same or a similar material to applicator 2708, and may transfer neurolytic agent to applicator 2708, by, e.g., capillary forces.

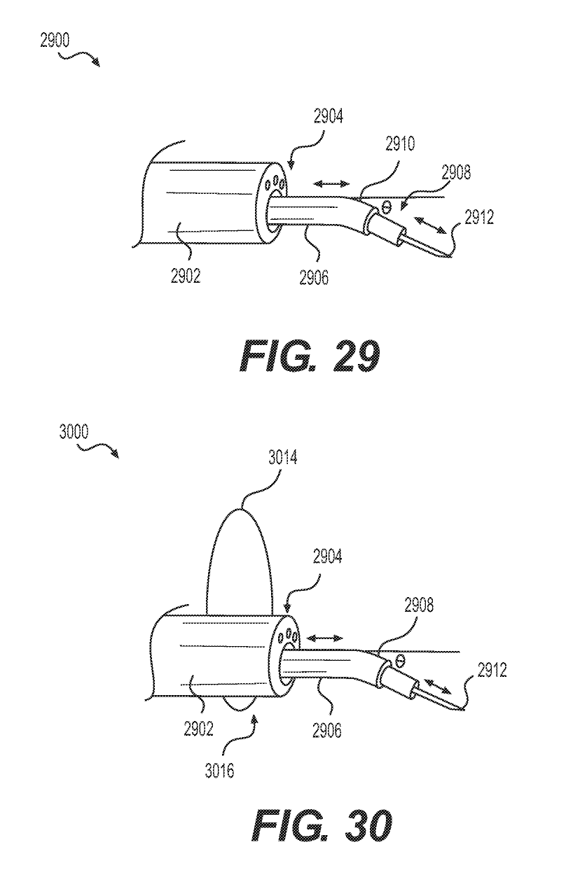

A medical device 2900 is shown in FIG. 29. Medical device 2900 may include a first elongate member 2902, such as, e.g., a bronchoscope. A second elongate member 2906 may extend from a distal end 2904 of first elongate member 2902. Second elongate member 2906 may include a bend 2910 at a distal end 2908. Bend 2910 may be offset from a longitudinal axis of medical device 2900 by an angle 8. A fluid delivery device 2912 may extend distally from bend 2910, and may be configured to transmit a fluid (e.g., a neurolytic agent) through an airway wall. Fluid delivery device 2912 may be substantially similar to fluid delivery device 1010 described with reference to FIG. 10.

Thus, in the embodiment of FIG. 29, fluid delivery device 2912 may be controlled to have a precise angle of insertion into the airway wall. This could be achieved by a guide lumen that exits the catheter at a fixed angle. In some embodiments, second elongate member 2906 and bend 2910 may be formed of, e.g., a shape memory material that may be substantially parallel to the longitudinal axis of medical device 2900 when constrained within first elongate member 2902. Second elongate member 2906 and bend 2910 then may take the form shown in FIG. 29 (e.g., may be angled at angle 8 offset from a longitudinal axis of medical device 2900) after extending distally from first elongate member 2902. Alternatively, fluid delivery device 2912 may be actuated to extend from bend 2910 at angle θ.

In some embodiments, the orientation of the beveled edge of the fluid delivery device 2912 may be controlled to control the angle of penetration. In some embodiments, the beveled edge may be configured to be parallel to the airway surface to provide a shallow penetration. This configuration may be useful when penetration between airway wall layers at a certain distance is desired (e.g. when penetration is desired between epithelial and smooth muscle layers).

Medical device 3000 depicted in FIG. 30 may be substantially similar to medical device 2900 except that medical device 3000 may additionally include expandable members 3014 and 3016. Expandable members 3014 and 3016 may be any suitable expandable members such as, e.g., balloons, stents, legs, prongs or the like. In some embodiments, one of expandable members 3014 and 3016 may expand to a greater extent than the other expandable member to position medical device 3000 closer to one radial portion of a lung airway than an opposing radial portion. For example, in the embodiment shown in FIG. 30, expandable member 3014 may expand more than expandable member 3016. Expandable member 3016 may also be configured to expand more than expandable member 3014 to position medical device 3000 closer to the opposing radial portion of the lung airway. Thus, in some embodiments, second elongate member 2906 may be rotatable, or medical device 3000 may include additional second elongate members 2906 with bends 2910 oriented in different directions. In some embodiments, one of expandable members 3014 and 3016 may be omitted entirely. In other embodiments, additional expandable members 3014 or 3016 may be included.

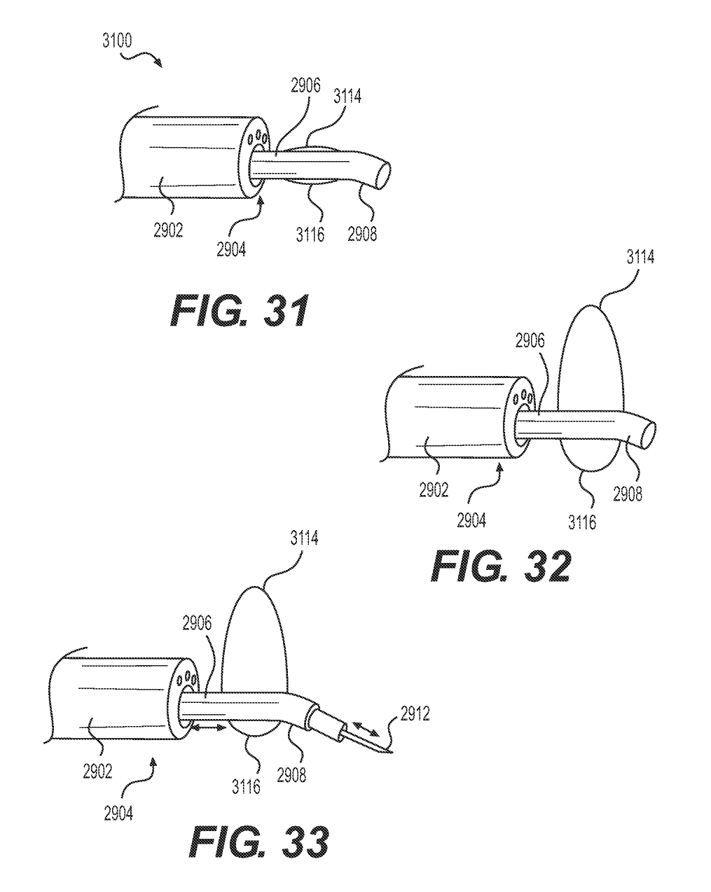

Medical device 3100 shown in FIGS. 31-33 may be substantially similar to medical device 3000, except that expandable members 3114 and 3116 may be disposed on second elongate member 2906 instead of on first elongate member 2902.

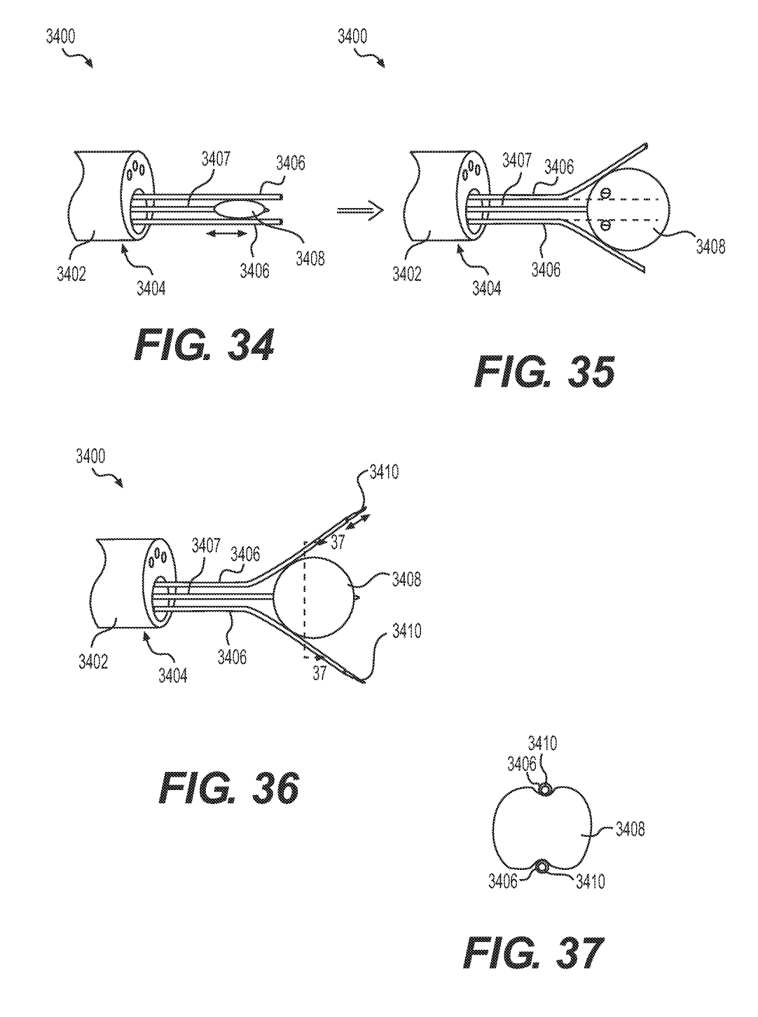

A medical device 3400 is shown in FIGS. 34-36. Medical device 3400 may include a first elongate member 3402, such as, e.g., a bronchoscope. One or more second elongate members 3406 may extend from a distal end 3404 of first elongate member 3402. A third elongate member 3407, such as, e.g., an inflation lumen, also may extend from distal end 3404, and may be disposed between second elongate members 3406. An expandable member 3408 may extend from the distal end of third elongate member 3407 between the distal ends of second elongate members 3406. After being extended from distal end 3404 in a collapsed configuration (shown in FIG. 34), expandable member 3408 may be moved to an expanded configuration shown in FIGS. 35 and 36. In the expanded configuration, expandable member 3408 may cause second elongate members 3406 to extend radially outward at an angle 8 offset from the longitudinal axis of medical device 3400. When expandable member 3408 is in the expanded configuration, the distal ends of second elongate members 3406 may be in contact with the surface of a lung airway wall. After expandable member 3408 has been moved to the expanded configuration, a fluid delivery device 3410 may extend distally from the distal end of each second elongate member 3406 to pierce through a surface of the airway wall. Fluid delivery device 3410 may be substantially similar to fluid delivery device 1010 described with reference to FIG. 10.

In some embodiments, second elongate members 3406 and third elongate member 3407 may be configured to translate axially with respect to one another, or second elongate members 3406 may be configured to translate axially with third elongate member 3407. In some embodiments, the various second elongate members 3406 may be configured to translate with respect to one another, and/or may be rotatable about their axes, to reach other target areas of a lung airway. In some embodiments, the distal ends of second elongate members 3406 may themselves be configured to penetrate tissue and/or deliver agent to nerves of the lung.

As seen in FIG. 37, second elongate members 3406 may be partially recessed within an outer surface of expandable member 3408 when expandable member 3408 is in the expanded configuration. Expandable member 3408 may be any suitable expandable member, such as, e.g., a balloon, stent, basket, or other suitable expandable member.

It should be noted that the embodiments of the present disclosure may alternatively have reservoirs or other sources of neurolytic agent disposed at or near the distal ends of the medical devices. Thus, delivery may come from the reservoir or source of neurolytic agent disposed at the distal ends instead of from the proximal ends of the medical device. For example, expansion of inflatable member 1008 can puncture a reservoir that is in communication with fluid delivery device 1010. Similarly, the expansion of inflatable member 1306 may puncture a reservoir in communication with pores 1308. In the embodiment of FIG. 15, the rolling of deployment member 1512 may cause the release of neurolytic agent from a reservoir. Further, some embodiments of the present disclosure may include identifying one or more nerves via a visualization method, such as, e.g., optical coherence tomography, and positioning a fluid delivery device adjacent to, or even within a nerve itself. For example, the myelin sheath of one or more nerve fibers may be punctured by a needle, and neurolytic agent may be delivered within the nerve fiber to reduce damage to adjacent, non-nerve tissues. Further, the various needles or fluid delivery devices configured to deliver neurolytic agent may additionally be configured to delivery another energy modality, such as, e.g., RF energy, to further damage or destroy nerve tissue.

Optical Energy

In some embodiments, optical energy (e.g., laser energy) may be applied to a lung airway in a controlled manner to damage afferent sensory nerves, efferent nerves, or the like. The optical energy may be configured to damage or destroy nerve function, including the ability of a targeted nerve to transmit signals.

Delivery of the optical energy may be through the right main bronchus, left main bronchus, or both, as treating only one of the right or left main bronchi may be sufficient for a significant reduction in bronchoconstriction and/or mucus production, as the right and left vagus nerves traverse along the right and left main bronchi, respectively.

Linking the delivery of optical energy with a detection system such as, e.g., an electrode mapping catheter, to a localized treatment location may allow for a more specific treatment to be applied, potentially reducing the damage to adjacent tissues. Other imaging procedures, such as, e.g., magnetic resonance imaging (MRI), diagnostic sonography, or other suitable imaging techniques also may be used.

The medical device of the present disclosure, e.g., medical devices 1000-1800, may deliver optical energy to locally ablate lung airway tissue or lung airway nerves. Delivery of optical energy may be via a minimally-invasive procedure that can ablate tissue located at a depth below the lung airway surface. The depth of energy delivery may be tuned and/or electronically adjusted. The adjustment may be determined by the power of the optical energy (e.g., laser) and the focal plane on which it is focused. That is, a medical device may not ablate tissue that is closest in proximity to the medical device, but may instead only ablate tissue that is a predetermined distance from the medical device. For example, the medical device may not ablate a closest 5 mm of tissue, while ablating tissue that is located between about 5 mm and 15 mm from the medical device, if desired. In one embodiment, the medical device may not ablate a closest 0.2 mm of tissue, while ablating tissue that is located about 0.2 mm and 2 mm from the medical device. It should be noted that other suitable ablation ranges may alternatively be utilized. In some embodiments, the medical device may ablate tissue at a depth beyond the surface of a lung airway wall so as not to irreversibly damage the epithelial layer. The preservation of the epithelial layer may avoid unnecessary trauma, inflammation, and/or mucus production, while maintaining the mucociliary clearance functions of the lung airways being treated.

In some embodiments, a location of the nerve(s) to be targeted in the airways may be determined by direct visualization, of, e.g., an anatomical structure, by ultrasound scanning/imaging, or by any other suitable means. Once a targeted nerve or treatment location is determined, the medical device may deliver optical energy to the targeted nerve or treatment location. In one embodiment, the targeted nerve or treatment location may be first detected by ultrasound scanning/imaging, and then optical energy may be delivered to a less than 360 degree circumference, e.g., a less than 90 degree circumference of the airway which corresponds with the target nerves or treatment location. In other embodiments, optical energy may be applied to an entire 360 degree circumference of the airway (e.g., in spiral treatment patterns). In some embodiments, little or no damage will be caused to the remaining circumference of the airways that are not targeted by the medical device. In some embodiments, the medical device may be capable of both imaging and delivering a therapy. Alternatively, the medical device may be configured for energy delivery around a larger circumference of the airway or esophagus, and may be directed at additional locations other than nerve tissue. In some embodiments, the medical device may direct optical energy toward smooth muscle tissue in the lung airways to achieve reduced bronchoconstriction (by e.g., scarring the smooth muscle tissue). In some embodiments, the medical device may direct optical energy toward tissues and body elements affecting other diseases such as, e.g., asthma, chronic cough, chronic bronchitis, and Cystic Fibrosis, where bronchoconstriction, mucus hypersecretion, and cough are also observed.

The medical devices also may be formed of a radiopaque material so that they can be visualized under fluoroscopic guidance, or otherwise include radiopaque or other imaging markers for guidance. The markers may be used to ensure that a correct direction of therapy is applied. In some embodiments, the medical device may be prevented from activating until the marker is appropriately positioned.

In some embodiments, medical devices may include one or more sensors to detect various parameters or anatomical structures. In one embodiment, the one or more sensors may include temperature sensors configured to detect a presence/amount of therapy delivered. In another embodiment, the one or more sensors may include structures within the lung airway configured to use Doppler ultrasound to detect blood vessels. In another embodiment, the one or more sensors may sense electrical measurement of nerve traffic that corresponds to an efficacy of the treatment. In another embodiment, the one or more sensors may include a vision system for direct observation. In yet another embodiment, the one or more sensors may include a force transducer, strain gauge, or similar sensor to measure radial force in the lung airways. One or more feedback mechanisms (e.g., PID, fuzzy logic, or the like) may be utilized to control the intensity of optical energy applied, and thus the extent of damage to lung tissues and lung nerves. In some embodiments, IR measurement, tissue optical parameter measurement (e.g., reflectance, color, scattering), direct temperature measurement (e.g., using thermocouples), or other suitable mechanisms may be utilized to measure the temperature change of lung tissue in response to the applied optical energy.

Controller 32 may be coupled to or otherwise include an optical energy source, such as, e.g., a holmium (Ho) laser source, a holmium:YAG (Ho:YAG) laser source, a neodymium-doped:YAG (Nd:YAG) laser source, a semiconductor laser diode, a potassium-titanyl phosphate crystal (KTP) laser source, a carbon dioxide (CO2) laser source, an Argon laser source, an Excimer laser source, a diode laser source, or another suitable laser source. In some embodiments, the laser source may be a laser diode. The laser diode may illuminate the tissue, and may be mounted at the distal end of a catheter or other suitable elongate member. In some embodiments, a high power (e.g., superluminescent) LED may be used in place of a laser source. In some embodiments, an intense, pulsed light source may be used in place of a laser source.

Carbon dioxide laser sources may be utilized in situations requiring tissue ablation with minimal bleeding. Argon laser sources may be utilized in situations requiring tissue ablation at short depth. Nd:YAG laser sources may be utilized in situations requiring deeper depth of penetration into tissue. Excimer laser sources may be utilized to remove very fine layers of tissue with little heating of surrounding tissue.

In some embodiments, the numerical aperture of optical energy emitted from the optical energy source may be between 0.1 and 0.4, or another suitable numerical aperture. The optical energy may be associated with a range of electromagnetic radiation from an electromagnetic radiation spectrum. In some embodiments, the delivered optical energy may be in a wavelength from about 300 nm to about 2100 nm, or may be another suitable wavelength.

Controller 32 may be configured to control (e.g., set, modify) a timing, a wavelength, and/or a power of the emitted optical energy. In some embodiments, the optical energy may have a power between 1 watt and 10 kilowatts, or may have another suitable power. In some embodiments, controller 32 may also be configured to perform various functions such as, e.g., laser selection, filtering, temperature compensation, and/or Q-switching.

Delivery Devices

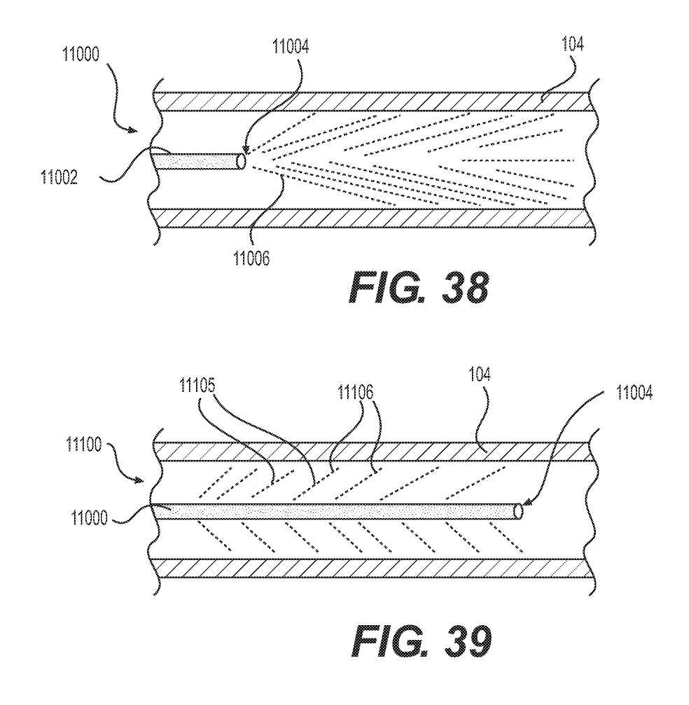

A delivery device, such as, e.g., a medical device 11000 shown in FIG. 38, may extend distally from a bronchoscopic member (e.g., a bronchoscope, not shown). Alternatively, medical device 11000 may extend from another suitable member or may be formed as a portion of the bronchoscopic member. In some embodiments, an elongate member 11002 may extend from a bronchoscopic member, guide catheter, or another suitable device. In some embodiments, elongate member 11002 may extend through one of a plurality of channels disposed through the bronchoscopic member. The channels may also allow for a variety of tools or fluids to be passed through the bronchoscopic member. Elongate member 11002 may thus translate within the corresponding lumen of the bronchoscopic member. In some embodiments, elongate member 11002 may be integrally formed with the bronchoscopic member. In some embodiments, an LED or other suitable delivery element may be integrally formed with a portion (e.g., the distal end) of the bronchoscopic member.

Elongate member 11002 may be configured to receive optical energy emitted (i.e., launched) from the optical energy source. In one embodiment, elongate member 11002 may be an optical fiber. Optical energy may be propagated through elongate member 11002 until the optical energy is transmitted from a distal end 11004 of elongate member 11002 toward, for example, a target treatment area within the lungs. That is, elongate member 11002 may act as a waveguide for the optical energy.

In some embodiments, elongate member 11002 may be a silica-based optical fiber and may include, for example, a fiber core, one or more cladding layers (e.g., a cladding layer disposed around the fiber core), a buffer layer (e.g., a buffer layer disposed around a cladding layer), and/or a jacket (e.g., a jacket disposed around a buffer layer). However, elongate member 11002 may additionally or alternatively include other suitable materials, and may be formed in other suitable configurations. At least a portion of the cladding layer(s), the buffer layer, and/or the jacket may be stripped from elongate member 11002 before a doped silica component is heat-fused to elongate member 11002. At least a portion of the doped silica component (e.g., the inner surface of the doped silica component) may have an index of refraction lower than an index of refraction associated with the outer portion of elongate member 11002. The doped silica component may be doped with a concentration of a dopant (e.g., a fluorine dopant, a chlorine dopant, a rare-earth dopant, an alkali metal dopant, an alkali metal oxide dopant, etc.) that may, at least in part, define the index of refraction of the doped silica component.

In some embodiments, the fiber core of elongate member 11002 may be formed of a suitable material for the transmission of optical energy from the optical energy source. In some embodiments, for example, the fiber core may be formed of silica with a low hydroxyl (OH—) ion residual concentration, or may be formed of another suitable material. The fiber core may be a multi-mode fiber core and can have a step or graded index profile. The fiber core may also be doped with a concentration of a dopant (e.g., an amplifying dopant).

In some embodiments, the cladding and/or buffer layers may be formed of acrylate or another suitable material. In one embodiment, the fiber core and/or cladding layer(s) may be formed of pure silica and/or doped with, for example, fluorine. In one embodiment, the cladding may be, for example, a single or a double cladding that can be made of a hard polymer, silica, or another suitable material. The buffer layer and/or jacket may be formed of a hard polymer such as, e.g., ethylene tetrafluoroethylene (ETFE), or of another suitable material.

In the embodiment shown in FIG. 10, elongate member 11002 may deliver optical energy 11006 from distal end 11004 in a divergent pattern to ablate large sections of a lung airway (such as, e.g., epithelial tissue 104) that are distal to distal end 11004. Optical energy 11006 may also be delivered in another suitable manner, such as, e.g., as a focused optical energy beam emitted from distal end 11004. Optical energy 11006 may have any suitable profile, such as, e.g., top-hat or Gaussian, among others.

A medical device 11100 is shown in FIG. 39. Medical device 11100 may include elongate member 11002 that delivers optical energy 11106 from a plurality of locations 11105 disposed along elongate member 11002. Locations 11105 may include portions of an optical fiber configured to emit optical energy 11106. For example, portions of elongate member 11002 other than locations 11105 may include an optically insulative material configured to block emission of optical energy. Locations 11105 may be configured in any suitable pattern. For example, a plurality of locations 11105 may be configured at regular or irregular intervals and patterns along elongate member 11002. In one embodiment, the plurality of locations 11105 may be arranged in a spiral shape to deliver a spiral treatment along epithelial tissue 104. In some embodiments, locations 11108 may be configured to emit optical energy 11106 that is focused, divergent, or in another suitable pattern. In one embodiment, locations 11105 may be configured to allow optical energy 11106 to be emitted circumferentially about elongate member 11002 (i.e., in a complete 360° ring about the longitudinal axis of elongate member 11002). Locations 11105 also may be configured to deliver a less than full-circumferential treatment (e.g., a partially-circumferential treatment). Partially-circumferential treatments of lung airways may be beneficial because, for example, if there is an inflammatory or other response that narrows the lumen of the airway, there may be a lower probability of complete closure when treatments are delivered in a non-fully circumferential pattern in a given axial location.

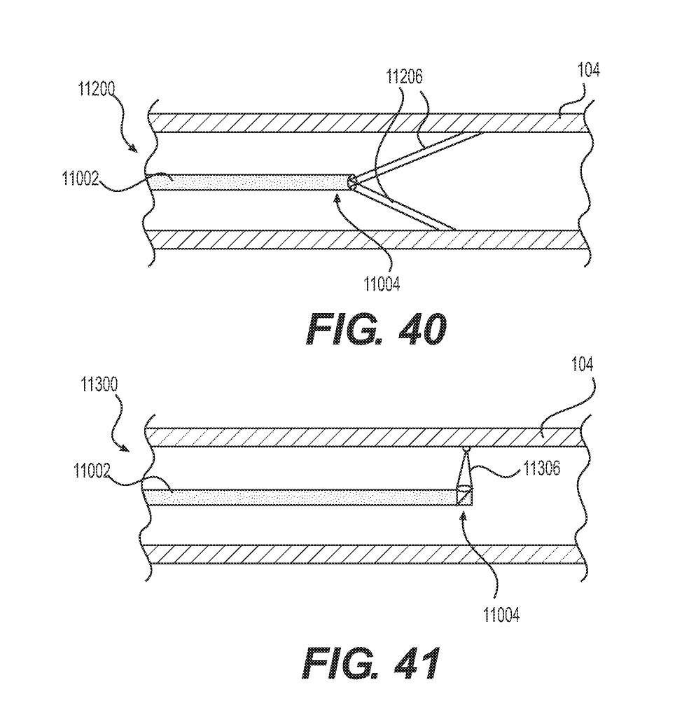

A medical device 11200 is shown in FIG. 40. Medical device 11200 may include elongate member 11002 that may deliver one or more focused optical energy beams 11206 from distal end 11004. While two optical energy beams 11206 are shown projecting from distal end 11004, any other suitable number of optical energy beams 11206 may be emitted. Optical energy beam 11206 may include an annular (e.g., ring-shaped) cross-section. However, other suitable beam profiles may also be utilized.

A medical device 11300 is shown in FIG. 41. Medical device 11300 may include elongate member 11002 that may deliver an optical energy beam 11306 at an angle that is offset from a longitudinal axis of elongate member 11002. In some embodiments, elongate member 11002 may deliver optical energy beam 11306 at an angle that is substantially orthogonal to the longitudinal axis of elongate member 11002. In some embodiments, the optical energy beam 11306 may be focused at a surface of epithelial tissue 1104. Optical energy beam 11306 may be a beam with an optical power per unit area that increases as the beam extends away from medical device 11300. That is, optical energy beam 11306 may narrow as it extends away from medical device 11300, increasing in intensity. Similar to the application of optical energy shown in FIG. 39, multiple optical energy beams 11306 may be delivered by medical device 11300 in any suitable pattern. For example, a plurality of optical energy beams 11306 may be configured at regular or irregular intervals and patterns along elongate member 11002. In one embodiment, the plurality of optical energy beams 11306 may be arranged in a spiral shape to deliver a spiral treatment along epithelial tissue 104. However, optical energy beam 11306 may alternatively be focused at other suitable locations as described with reference to other embodiments of the present disclosure.

A medical device 11400 is shown in FIG. 42. Medical device 11400 may include elongate member 11002 that may deliver an optical energy beam 11406 in a substantially similar manner as described with reference to FIG. 41, except that optical energy beam 11406 may be focused at a depth (e.g., two mm or another suitable depth) beyond the surface of epithelial tissue 104.

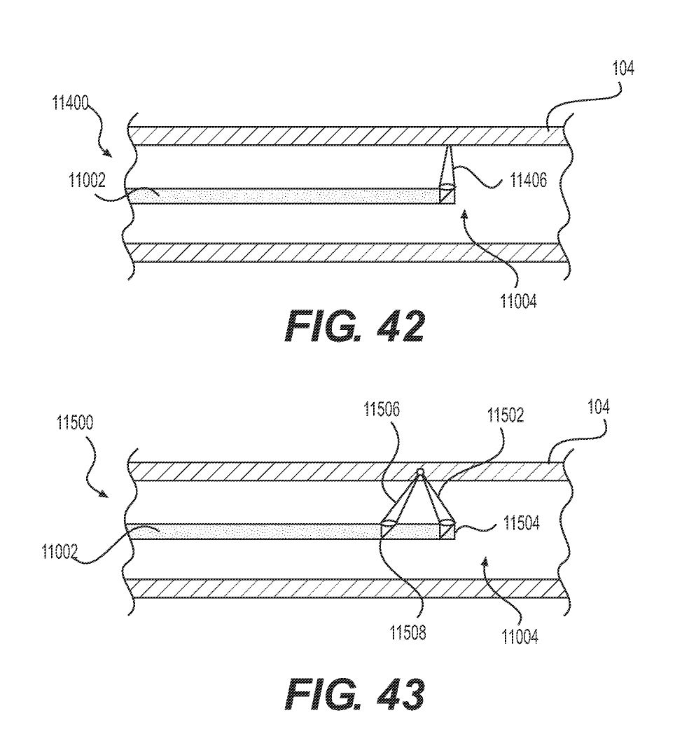

A medical device 11500 is shown in FIG. 43. Medical device 11500 may include elongate member 11002 that may deliver a plurality of optical energy beams to a single location beyond the surface of airway 108. For example, a first optical energy beam 11502 may be emitted from a first location 11504. In the embodiment shown in FIG. 43, first location 11504 is disposed at distal end 11004 of elongate member 11002, but may be located at any other suitable location. A second optical energy beam 11506 may be emitted from a second location 11508 that is longitudinally offset from the first location. That is, the first and second optical energy beams 11502, 11506 may be emitted from longitudinally offset locations, but focused to ablate the same location or region. In some embodiments, first and second optical energy beams 11502, 11506 may be focused at a surface of epithelial tissue 104. While two optical energy beams 11502, 11506 are shown to be focused toward the same location in FIG. 43, any additional number of optical energy beams may be directed to the same location. In some embodiments, multiple pairs or pluralities of optical energy beams may be directed simultaneously toward different locations along the epithelial tissue 104. The focusing of multiple optical energy beams to a single location may reduce the time required to ablate tissue to a desired level, potentially reducing collateral heat damage to surrounding tissues that are not intended for ablation.

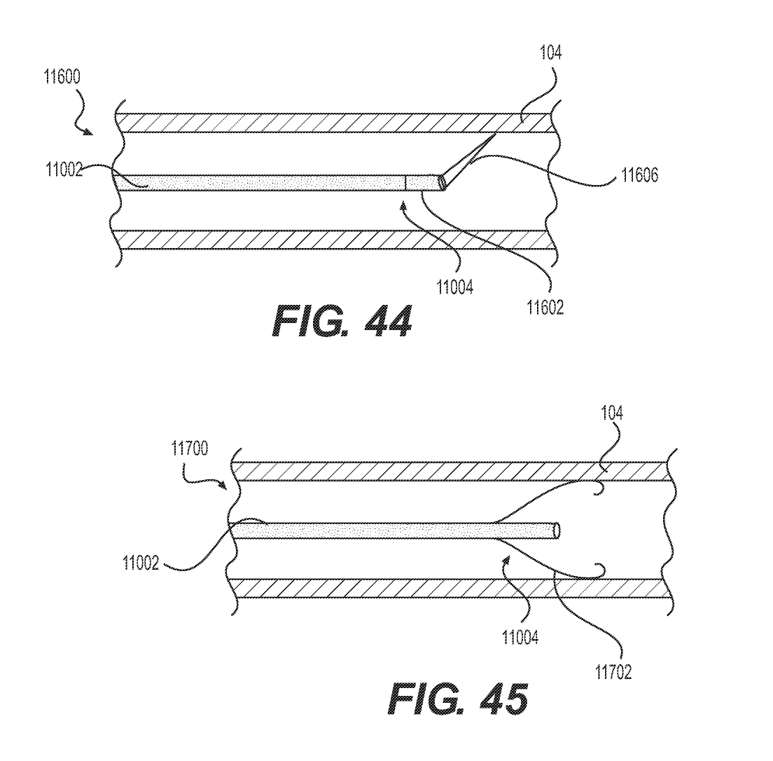

A medical device 11600 is shown in FIG. 44. Medical device 11600 may include elongate member 11002, and a rotating tip 11602 disposed at distal end 11004 of elongate member 11002. Rotating tip 11602 may be rotatable about a longitudinal axis of elongate member 11002 by any suitable mechanism, and may be configured to deliver an optical energy beam 11606. In some embodiments, rotating tip 11602 may be configured to deliver optical energy beam 11606 at an angle offset from a longitudinal axis of elongate member 11002. In some embodiments, the delivery of optical energy beam 11604 may remain constant during rotation of rotating tip 11602 to achieve full circumferential delivery of optical energy to airway wall 108. In some embodiments, the delivery of optical energy beam 11606 may be partially interrupted during the rotation of rotating tip 11602 to achieve a patterned treatment. In some embodiments, multiple optical energy beams 11604 may be delivered from rotating tip 11602. The multiple optical energy beams 11606 may be focused toward the same or to different locations. In some embodiments, elongate member 11002 may include one or more rotating sections (not shown) disposed at varying longitudinal positions. In some embodiments, a spiral treatment pattern may be obtained by pushing or pulling elongate member 11002 while rotating the rotating tip 11602.

A medical device 11700 is shown in FIG. 45. Medical device 11700 may include elongate member 11002, and one or more guide members 11702 disposed at distal end 11004 of elongate member 11002. Guide members 11702 may be wires or another suitable guide member configured to locate elongate member 11002 within the airway defined by epithelial tissue 104. In some embodiments, guide wires 11702 may be configured to locate elongate member 11002 at a middle portion of the airway, e.g., a center of the airway. In some embodiments, various guide members 11702 may be separately configured to locate elongate member 11002 closer to a wall of epithelial tissue 104 of the airway as compared to another wall of epithelial tissue 104 (e.g., separate guide members 11702 may be spring-like members having different biases). In the embodiment shown in FIG. 45, two guide members 11702 are shown, although any other suitable number of guide members 11702 may also be utilized. Guide members 11702 may have a lubricious coating to facilitate the movement of medical device 11700 through epithelial tissue 104 (e.g., in a longitudinal and/or circumferential direction). Guide members 11702 may be biased into an expanded configuration as shown in FIG. 45, or may be separately and reciprocally actuatable from a collapsed configuration (not shown) to the expanded configuration. Medical device 11700 may be configured to deliver any suitable optical energy beam described with reference to medical devices 11000-11600, and 11800. Further, guide members 11702 may be combined with a delivery or medical device configured to deliver any other suitable energy modality or substance, such as, e.g., a neurolytic agent, cryotherapy, acoustic energy, RF energy, microwave energy, or another suitable energy modality to promote the rapid application of energy to the lung airways.

One or more guide members 11702 may be located at additional or alternative locations along elongate member 11002, such as, e.g., at other longitudinal and/or circumferential locations. In some embodiments, guide members 11702 may be replaced or combined with suitable anchoring members, such as, e.g., expandable baskets, balloons, springs, or other suitable anchoring members.

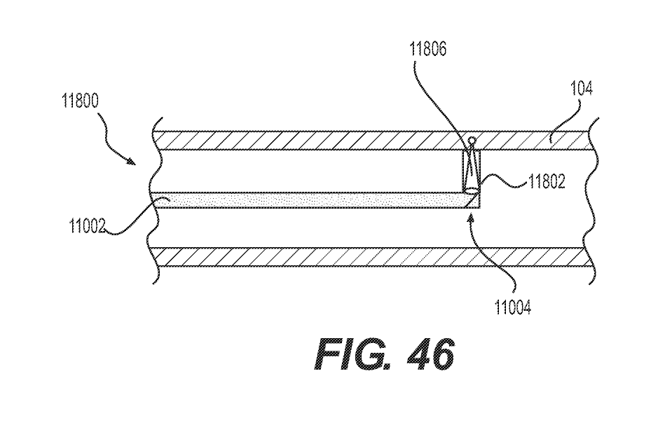

A medical device 11800 is shown in FIG. 46. Medical device 11800 may include elongate member 11002, and one or more guide members 11802 disposed at distal end 11004 of elongate member 11002. Guide member 11802 may be a hollow tube configured to separate elongate member 11002 from a surface of epithelial tissue 104 by a predetermined distance. The predetermined distance may correspond to the length of guide member 11802, and may allow an operator to locate medical device 1800 at an optimal delivery location for an optical energy beam 11806. Optical energy beam 1806 may be configured to travel through guide member 11802. In some embodiments, guide member 11802 may be hollow, but closed at a distal end, allowing a cooling fluid to circulate through a lumen of guide member 11802. The cooling fluid may be configured to prevent damage to, e.g., a surface of epithelial tissue 104 when it is desired to only ablate tissue located beyond the surface of epithelial tissue 104. In some embodiments, guide member 11802 may be movable between a collapsed configuration (not shown) and an expanded configuration shown in FIG. 46 by suitable mechanisms (e.g., guide member 11802 may be telescopic). In one embodiments, guide member 11802 may articulate from a position generally along or parallel to the longitudinal axis of elongate member 11002 to a position transverse (e.g., perpendicular to) the longitudinal axis.

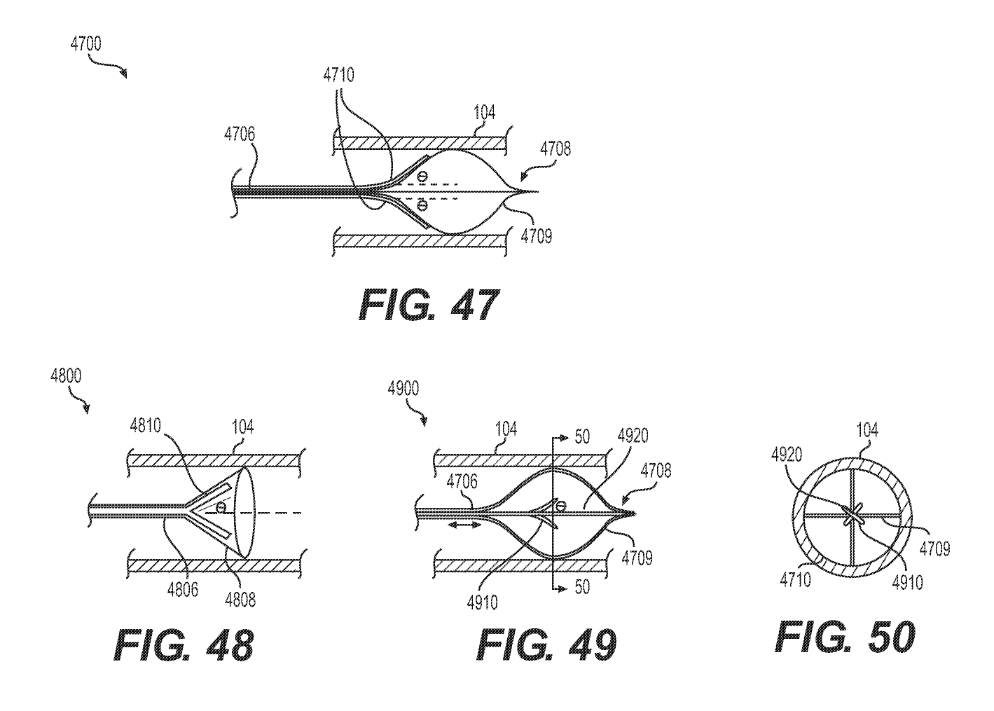

A medical device 4700 is shown in FIG. 47. Medical device 4700 may include elongate member 4706, and an expandable member (e.g., a basket) 4708 having a plurality of radially spaced legs 4709 disposed at the distal end of elongate member 4706. However, expandable member 4708 may be any other suitable expandable member, such as, e.g., a stent or balloon. One or more optical elements 4710 may be disposed on a partial length of the one or more legs 4709, and may be configured to direct optical energy toward epithelial tissue 104. Optical elements may be disposed on a curved surface of legs 4709 when legs 4709 are in an expanded configuration such that a distal end of optical elements 4710 are directed toward epithelial tissue 104 at angle 8. For example, a first optical element 4710 may be coupled to a first leg 4709, and a second optical element 4710 may be coupled a second leg 4709. Optical elements 4710 may be configured to direct optical energy at an angle 8 offset from the longitudinal axis of medical device 4700. In some embodiments, all of legs 4709 may be expanded or activated together. Alternatively, each leg 4709 may be independently expanded or activated. In some embodiments, different legs 4709 may be formed in different shapes (e.g., different pre-bent shapes) to direct optical elements 4710 to different axial locations along a lung airway.

A medical device 4800 is shown in FIG. 48. Medical device 4800 may include elongate member 4806, and an expandable member (e.g., a cone) 4808 disposed at the distal end of elongate member 4806. While depicted as conical, expandable member 4808 may have any other suitable shape. One or more optical elements 4810 may be disposed within cone 4808 and oriented at an angle 8 offset from the longitudinal axis of medical device 4800 to direct optical energy toward epithelial tissue 104. In some embodiments, optical elements 4810 alternatively may be disposed on an outer surface of expandable member 4808. In some embodiments, an asymmetric expandable member 4808 may be configured to direct different optical elements 4810 to different axial locations along a lung airway.

A medical device 4900 is shown in FIGS. 49 and 50 that is similar to medical device 4700 except that optical elements 4910 may be coupled to a central elongate member 4920 that is disposed between legs 4709. Central elongate member 4920 may be a pull wire or other suitable member that extends through elongate member 4706 toward a distal tip of expandable member 4708. In the configuration shown in FIGS. 49-50, optical elements 4910 may be biased such that distal ends of optical elements 4910 are directed toward epithelial tissue 104 at angle 8.

Optical energy beams of the present disclosure may also be used in combination with nerve-specific or tissue-specific probes (e.g., fluorescent probes) including, but not limited to, cationic styryl dyes, low molecular weight dextrans (e.g., ≧10 kD), peptides, antibodies, or other suitable probes. In some embodiments, the probe may be Biocytin (e.g., ∈-biotinoyi-L-lysine, and biotin ethylenediamine derivatives), lipophilic tracers, Carbocyanine dyes, Dil and its derivatives (e.g., Dil-C12), Fluoro-Gold, and Hydroxystilbamidine, among other suitable probes. The fluorescent probe may be injected intravenously or locally into the subepithelial space in the region of the lung to be treated, where the fluorescent probe may specifically adhere to a targeted tissue a suitable biological or chemical linker. In some embodiments, probes could be delivered intravenously, or injected directly into the airway wall (sub-epithelial space) using a microneedle on a catheter via a bronchoscope. Probes could also be sprayed or topically applied to the airway wall, or applied to the exterior or the airway wall through insertion of an injection catheter via the chest wall.

The targeted nerves and/or tissue, now containing the fluorescent probe, may be exposed to the optical energy through an appropriate wavelength filter to excite the fluorescent probe. The excited fluorescent probes may result in localized damage and/or necrosis to the nerve fibers/tissue, while having a therapeutically negligible effect on adjacent non-neural/non-target tissue. In addition, for deeper penetration, selective photothermolysis can be used where, in a similar fashion, a chromophore is conjugated to a molecule that is specifically taken up by neuron cells. The exposure of the attached chromophore to optical energy of a specific wavelength band may cause higher absorption than in neighboring, non-chromophore containing tissue. Thus, the neural having an attached chromophore may be damaged, while leaving neighboring tissue unharmed. In one embodiment, Procion Blue conjugated to wheatgerm agglutinin may be delivered to a target nerve, and exposed to a laser of wavelength 600 nm, an energy density of 50 J/cm2, and pulse duration of 1 μs, to specifically ablate a target nerve.

In some embodiments, an interface between the medical devices of the present disclosure and the lung airways may be formed or bridged with an amount of saline or another suitable liquid with similar refractive index to the fiber and the tissue. The formation of this interface may enhance light coupling between the medical devices and the lung airway walls. The formation of the interface may reduce the focusing and scattering variation caused by undulations in the airway surface. In some embodiments, a compliant balloon may be filled with saline or another liquid having a similar refractive index, and optical energy may be delivered though the saline toward the lung airways.

Cryo Energy

In some embodiments, a cooling element or substance may be applied to the airway in a controlled manner to damage nerves of the lung, e.g., C-fibers, RAR receptors, SAR receptors, or the like. The cooling element or substance may be configured to damage or destroy nerve function, including the ability of a targeted nerve to transmit signals.

Delivery of the cooling element or substance may be through the right main bronchus, left main bronchus, or both, as treating only one of the right or left main bronchi may be sufficient for a significant reduction in bronchoconstriction and/or mucus production, as the right and left vagus nerves traverse along the right and left main bronchi, respectively.

Linking the delivery of a cooling element or substance with a detection system such as, e.g., an electrode mapping catheter, to a localized treatment location may allow for a more specific treatment to be applied, potentially reducing the damage to adjacent tissues.

In some embodiments, cooling may be accomplished for example, by injecting a cold fluid into lung parenchyma or into the lung airway being treated, where the airway is proximal, distal, or circumferentially adjacent to the treatment site. In some embodiments, the fluid may be water, saline, liquid nitrogen, carbon dioxide, hydrofluorocarbons (e.g., Freon), refrigerants, or the like. Some or all of these fluids may be injected into a device (e.g., a balloon catheter) that conducts heat through the balloon. The fluid may be injected into treatment regions within the lung while other regions of the lung are ventilated by a gas. Or, the fluid may be oxygenated to eliminate the need for alternate ventilation of the lung. Upon achieving the desired reduction or stabilization of temperature, the fluid may be removed from the lungs. In the case where a gas is used to reduce temperature of the lungs, the gas may be removed from the lung or allowed to be naturally exhaled. One benefit of reducing or stabilizing the temperature of the lung may be to prevent excessive destruction of the tissue, or to prevent destruction of certain types of tissue such as the epithelium, or to reduce the systemic healing load upon the patient's lung.

Delivery Devices

As shown in FIG. 51, a medical device 21000 may include an elongate member 21002 extending from a proximal end (not shown) toward a distal end 21004. A cooling member 21006 may extend from the distal end 21004 of elongate member 21002. Alternatively, cooling member 21006 may be disposed at another location on elongate member 21002. In some embodiments, multiple cooling members 21006 may be disposed on elongate member 21002.

In one embodiment, cooling member 21006 may be an expandable or inflatable member (e.g., a balloon) that is configured to be reciprocally movable between an expanded/inflated configuration and an unexpanded/deflated configuration. In such embodiments, a cooled substance (e.g., a gas or liquid) may be delivered to cooling member 21006 by a lumen disposed through or along elongate member 21002. In some embodiments, the cooled substance may include one or more of water, saline, liquid nitrogen, carbon dioxide, freons, refrigerants, or the like.

Alternatively, cooling member 21006 may be any suitable member configured to reduce the temperature of tissue. In some embodiments, cooling member 21006 may not be expandable or inflatable. In some embodiments, cooling member 1006 may be a cryoprobe, lumen, manifold, tube, or other suitable arrangement through which a cooled substance passes through.

In some embodiments, cooling member 21006 or another portion of medical device 21000 may include a guidance/anchoring mechanism configured to direct medical device 21000 via, e.g., direct visualization, ultrasound (e.g., EBUS), CT-guidance, Optical Coherance Tomography (OCT), or electrical characterization of nerve location (e.g., by sensing electrical nerve or muscle signals). The guidance mechanism may also include a detector for detecting afferent sensory nerves, efferent nerves, or other nerves. Alternatively or additionally, all or a portion of medical device 21000 may be formed of a radiopaque material so that it can be visualized under fluoroscopic guidance, or may otherwise include radiopaque or other imaging markers for guidance. Markers may be used to ensure that a correct direction of therapy is applied under direct visualization through a bronchoscope, endoscope, or other delivery device or under fluoroscopic guidance. In some embodiments, the guidance mechanism and/or cooling member 21006 may be prevented from activating until the marker is appropriately positioned.

In some embodiments where cooling member 21006 is not inflatable or expandable, the guidance mechanism may be configured to be reciprocally movable between an unexpanded/deflated configuration and an expanded/inflated configuration. The guidance mechanism may be inflated by any suitable mechanism. For example, the guidance mechanism may be inflated by an infusion of saline, air, or other gases or liquids from a lumen of elongate member 21002. When the guidance mechanism is inflated, it may act as an anchor for medical device 21000 within lung airways. The guidance/anchoring mechanism may also be combined with any other embodiment described in the present disclosure.

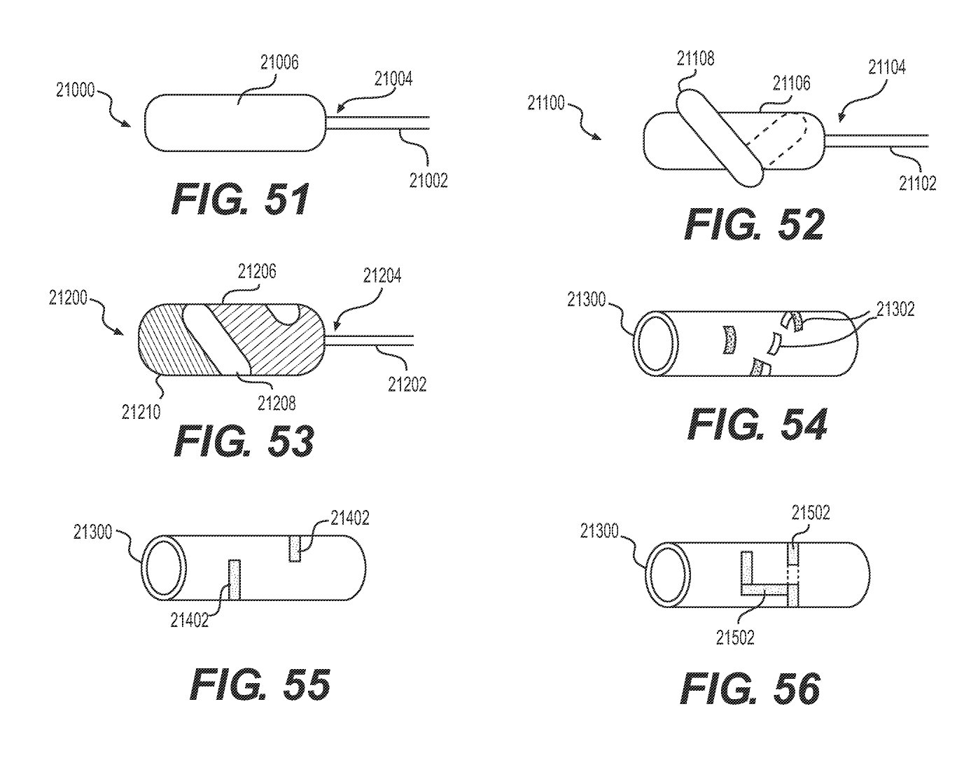

A medical device 21100 is shown in FIG. 52. Medical device 21100 may include an elongate member 21102 extending from a proximal end (not shown) toward a distal end 21104. A cooling member 21106 may extend from the distal end 21104 of elongate member 21102, or may be otherwise arranged in the same manner as described with reference to cooling member 21006 of FIG. 51.

Cooling member 21106 may be substantially similar to cooling member 21006 except that cooling member 21106 may additionally include a raised portion 1108 disposed on an outer surface of cooling member 21106. Raised portion 21108 may be formed of the same material as cooling member 21106, or may be formed of another suitable material. Raised portion 21108 may be configured to allow an operator of medical device 21100 to cool selective portions of tissue that contact raised portions 21108. As shown in FIG. 52, raised portion 21108 is depicted as a spiral. However, raised portion 21108 may be disposed in any other suitable shape, such as, e.g., axial, circumferential, zig-zagged, or the like. In some embodiments, a plurality of raised portions 21108 may be arranged in columns and rows, or in another suitable arrangement. In some embodiments, multiple raised portions 21108 may be disposed on the same cooling member 21106, or multiple cooling members 21106 each having one or more raised portions 21108 may be disposed on elongate member 21102. In some embodiments, raised portion 21108 may include many discrete portions arranged in a pattern. For example, raised portion 21108 may include multiple raised portions that are arranged in a spiral, axial, circumferential, zig-zag, or another suitable arrangement. In some embodiments, multiple rows of raised portions 21108 may be disposed along cooling member 21106. The pattern of raised portion 21108 may allow for a treatment that damages nerves and/or tissue, but still maintains mucociliary function within lung airways. Alternatively, member 21106 may be insulated and member 1108 may serve as the cooling member alone, such that the temperature on the outer surface of member 1106 is greater than that on the outer surface of member 21108. It should be noted that the temperature of cooling members 21106 does not have to be low enough to cause the apoptosis or necrosis to occur, but the temperature of member 21108 does need to be adequately low.

If medical device 21100 is a balloon, raised portion 21108 may communicate with a remainder of cooling member 21106 to receive fluid. In an alternative embodiment, cooling fluid may instead flow from a lumen of elongate member 21102 directly into only raised portion 21108, and another lumen of elongate member 21102 may deliver an inflation fluid to a remainder of cooling member 21106. In some embodiments, fluid delivered to the remainder of cooling member 21106 may be warm, or at a relatively higher temperature than the cooling fluid, to reduce the damage to the epithelium adjacent to the non-raised portions of cooling member 21106. The cooled raised portion 21108 may be insulated from the warmed, remaining regions of cooling member 21106 in some embodiments.

A medical device 21200 is shown in FIG. 53. Medical device 21200 may include an elongate member 21202 extending from a proximal end (not shown) toward a distal end 21204. A cooling member 21206 may extend from the distal end 21204 of elongate member 21202, or may be otherwise arranged in the same manner as described with reference to cooling member 1006 of FIG. 10.

Cooling member 21206 may be substantially similar to cooling member 21006 except that cooling member 21206 may include active regions 21208 and insulated regions 21210. Active regions 21208 may be configured to reduce tissue temperature upon contact, while insulated regions 21210 may have a negligible effect on tissue temperature upon contact. Active regions 21208 are depicted in FIG. 53 as having a spiral shape, but may have any other suitable shape, such as those described with reference to raised portions 21108 of FIG. 52. It should be noted that all inflatable members (e.g., balloons) described in the present disclosure may include one or more temperature sensors coupled to a controller (e.g., controller 32). The temperatures sensors may be disposed inside or outside the surface of the inflatable member to provide temperature information to controller 32, for use in, e.g., suitable temperature feedback control mechanisms.

Insulated regions 21210 may be formed of an insulated material, such as, e.g., polymer (e.g., PTFE, PET, or polyamides), silicone, foamed polymer or air-filled cavity, woven polymer, reflective coating on the polymer (foil, sputtered gold, chrome, aluminum or other metal), or any material that results in the surface temperature of insulated regions 21210 being greater than that of the surface temperature of cooling member 21208. In some embodiments, the separation of insulated regions 21210 and active regions 21208 may be achieved by the routing of cooled substances through cooling member 21206 in specific patterns, or by other suitable mechanisms. Channels for fluid flow may be formed from microtubing or laser etching of polymers, for example. In some embodiments, insulated regions 21210 may be formed by applying a low-conductive substance to cooling member 21206 in a desired pattern. In an alternative embodiment, cooling member 21206 may be formed of multiple materials having varying conductivities. In another alternative embodiment, cooling fluid may instead flow from a lumen of elongate member 21202 directly into active regions 21208, and another lumen of elongate member 21102 may deliver an inflation fluid to insulated regions 1210. The inflation fluid may be warmed as described above with reference to FIG. 52.

In some embodiments, medical device 21200 may be formed by two balloons. For example, an inner balloon may be disposed within an outer balloon. The inner balloon may be sealed so as to be airtight, so that when filled with a cooling fluid (e.g., liquid nitrogen), it does not leak. The outer balloon may have insulative properties equal to or greater than that of the cooling properties of the cooling fluid flowed through the inner balloon. To form an active region 21208, the material of the outer balloon may be ablated away in that region or regions. Referring to FIG. 53, active regions 21208 may only have a single-balloon layer (the inner balloon) between the cooling fluid and the airway wall. Insulated regions 21210, however, may have two layers of balloon material (both the inner and outer balloons) between the cooling fluid and the airway wall. The inner and outer balloons may be formed of any suitable material, such as, e.g., PET, Nylon, and PEBAX, among others. Note that in the example above, the ablated regions representing the treatment zones could alternatively be on the inner balloon instead of the outer balloon. In an alternative embodiment, medical device 21200 may be formed of a single balloon having variable thicknesses. For example, active regions 21208 may have microlayers of material removed. Alternatively or additionally, the single balloon may have layers of material added to the balloon to form insulated regions 21210.

FIGS. 54-56 show exemplary treatment patterns along a lung airway 21300. FIG. 54 depicts airway 21300 with multiple treatment locations 21302 that are together arranged along a spiral path. FIG. 55 depicts airway 21300 with two treatment locations 21402 that are arranged on opposite radial portions of airway 21300. While only two treatment locations are depicted in FIG. 55, any other suitable number of locations may be disposed in any other suitable spacing arrangement along airway 21300. FIG. 56 depicts airway 21300 with multiple treatment locations 21502 that are arranged in a zig-zag pattern. In alternative embodiments, airway 21300 may be treated in any suitable pattern that minimizes the effect of short-term (acute) lung function and mucociliary clearance.

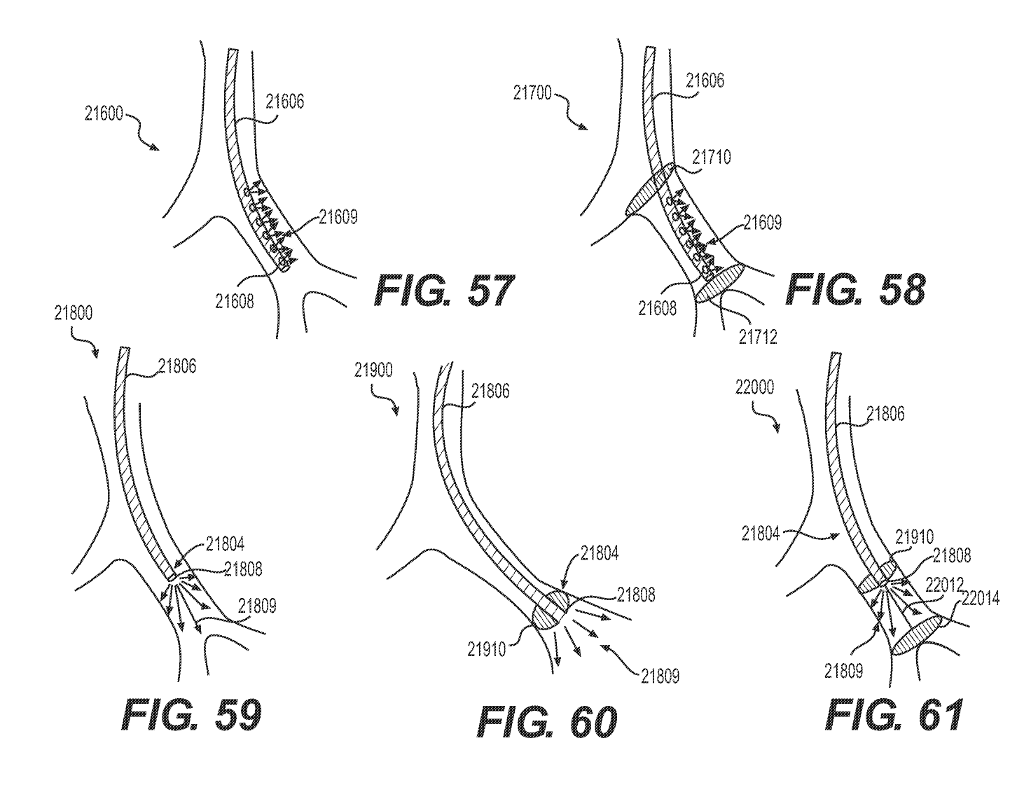

A medical device 21600 is shown in FIG. 57. Medical device 21600 may include an elongate member 21606 that may extend from a distal end of a bronchoscope (not shown) or another suitable elongate member. Elongate member 21606 may have a tubular structure defining a circular cross-section, or another suitable cross-section such as, e.g., elliptical, rectangular, variable, or the like. Elongate member 21606 may be formed from any suitable flexible and/or biocompatible material, including, but not limited to, metals, polymers, alloys, or the like. In some embodiments, elongate member 21606 may be formed from one or more of nitinol, silicone, or the like. According to one embodiment, the material may exhibit sufficient flexibility to be maneuvered through the lung airways without causing any injury to the surrounding tissue. That is, elongate member 21606 may be atraumatic so as not puncture lung tissue upon contact.

Medical device 21600 may include one or more openings 21608 disposed along elongate member 21606. Each opening 21608 may be coupled to a source of a cooled substance (not shown), and may be configured to deliver an amount of the cooled substance as a cryospray 21609. Each opening 21608 may be configured to deliver cryospray 21609 in any suitable manner, such as, e.g., as sprays, jets, trickles, streams, or the like. In some embodiments, a given opening 21608 may be configured to deliver cryospray 21609 in a proximal direction, a distal direction, or in a direction that is substantially orthogonal to the longitudinal axis of elongate member 21606. In some embodiments, openings 21608 may be configured to deliver different substances (e.g., via different lumens within elongate member 21606), so that a user can selectively deliver substances to desired treatment sites, i.e., a first lumen may correspond to a single or multiple openings 21608, while a second lumen may correspond to one or more other openings 21608. Medical device 21600 also may include an exhaust lumen (not shown) to remove cryospray from the airway. In some embodiments, the exhaust lumen may be disposed at the distal end of medical device 21600 or distal to the distal end.

Cryospray 21609 may lower the temperature of lung airway tissue to, e.g., a temperature range of about −5° C. to −50° C., although other suitable temperatures are also contemplated. In some embodiments, a target treatment temperature may be selected such that the cells of the lung airway tissue and nerves may be subject to necrosis but not apoptosis. In some embodiments, causing cell death by necrosis may hinder the regeneration of nerves as opposed to causing cell death by apoptosis. Alternatively, it may be desirable to cause cell death by apoptosis as opposed to necrosis in order to limit the severity of effects on residual tissues.

A medical device 21700 is depicted in FIG. 58 that is substantially similar to medical device 21600 described with reference to FIG. 57, except that medical device 21700 may also include a proximal inflatable or expandable member 21710 disposed proximal to openings 21608 along elongate member 21606. Proximal expandable member 21710 may be inflated by an infusion of saline, air, or other gases or liquids from a lumen of elongate member 21606. In some embodiments, the inflation gas or gases may be heated to help prevent damage to peripheral tissues caused by the reduced temperature of the airway. A distal inflatable or expandable member 21712 may be disposed distal to openings 21608 along elongate member 21606. Distal expandable member 21712 may be inflated in a substantially similar manner as proximal expandable member 21710. Each of proximal expandable member 21710 and distal expandable member 21712 may be movable between a deflated or collapsed configuration (not shown), and an inflated or expanded configuration (shown in FIG. 58). Expandable members 21710 and 21712 may be balloons or similar expandable members configured to create a seal around a treatment region. Proximal expandable member 21710 and distal expandable member 21712 may allow an operator to deliver cryospray 21609 to an enclosed region along a given lung airway. In an alternative embodiment, expandable members 21710 and 21712 may not be configured to form a seal around a treatment region, and may be formed as expandable baskets, or as other suitable expandable members to, e.g., anchor elongate member 21606 in place. In some embodiments, expandable members 21710 and 21712 may be independently expandable/inflatable. Medical device 21700 may include an exhaust lumen for removing cryospray from the airway, which may also help equalize the pressure within the region sealed between expandable members 21710 and 21712 when cryospray 21609 is introduced. Thus, in some embodiments, medical device 21700 may include four lumens (e.g., a coolant entry lumen, a coolant exhaust lumen, and lumens for expandable members 21710 and 21712). Thus, medical device 21700 may allow for pre- and post-treatment aspiration or cleaning of airway surfaces in the region sealed between expandable members 21710 and 21712.

In an alternative embodiment, medical device 21700 may include only one of proximal expandable member 21710 and distal expandable member 21712. In yet another alternative embodiment, medical device 21700 may include additional expandable members and openings along elongate member 21706 such that a plurality of treatment regions can be formed in an airway by medical device 21700. For example, a medical device may include a first, second, and third expandable member. A first set of openings may be disposed between the first and second expandable members, and a second set of openings may be disposed between the second and third expandable members. Medical device 21700 may be configured to deliver fluid to a selected set of openings independent of the other sets of openings.

A medical device 21800 is shown in FIG. 59. Medical device 21800 may include an elongate member 21806 that extends from a distal end of a bronchoscope (not shown) or another suitable elongate member. Medical device 21800 may include one or more openings 21808 disposed at a distal end 21804 of elongate member 21806. Opening 21808 may be coupled to a source of a cooled substance (not shown), and may be configured to distally deliver an amount of the cooled substance as a cryospray 21809. Opening 21808 may be configured to deliver cryospray 21809 in any suitable manner, such as, e.g., as sprays, jets, trickles, streams, or the like. Cryospray 21809 may be substantially similar to cryospray 21609 described with reference to FIG. 57. Medical device 21800 may include an exhaust lumen (not shown) that is substantially similar to the exhaust lumen described with reference to FIG. 57.

A medical device 21900 is shown in FIG. 60 that is substantially similar to medical device 21800 described with reference to FIG. 59, except that medical device 21900 may include a distal inflatable or expandable member 21910 disposed at distal end 21804 of elongate member 21806. When inflated, expandable member 21910 may form a seal in the lung airway, preventing cryospray 21809 from travelling to locations proximal to expandable member 21910. Thus, while expandable member 21910 is in the inflated or expanded configuration, cryospray 21809 may be delivered only to locations that are distal (or downstream) to expandable member 21910. Expandable member 21910 may also serve to anchor medical device 21900 within a lung airway. Medical device 21900 may include an exhaust lumen (not shown) that is substantially similar to the exhaust lumen described with reference to FIG. 57.

A medical device 22000 is shown in FIG. 61 that is substantially similar to medical device 21900, except that medical device 22000 may further include a distal support 22012 and a second expandable member 22014, both extending distally from elongate member 21806. Distal support 22012 may extend distally from the distal end of elongate member 21806, and second expandable member 22014 may be disposed on the distal end of distal support 22012. Expandable members 21910 and 22014 may be balloons or similar expandable members configured to create a seal around a treatment region. Thus, expandable members 21910 and 22014 may allow an operator to achieve a specificity of treatment along a portion of a given lung airway for delivering cryospray 21809. Expandable members 21910 and 22014 may be inflated in a substantially similar manner as expandable member 21710 described with reference to FIG. 58. Distal support 22012 may be configured to translate with respect to distal end 1804 to vary the length of a treatment region. Distal support 22012 may include a lumen to deliver inflation fluid to expandable member 22014. Medical device 22000 may include one or more lumens (not shown) that are substantially similar to the lumens described with reference to FIG. 58. Thus, medical device 22000 may allow for pre- and post-treatment aspiration or cleaning of airway surfaces in the region sealed between expandable members 21910 and 22014.

In an alternative embodiment, expandable members 21910 and 22014 may not be configured to form a seal around a treatment region, and may be formed as expandable baskets or as other suitable expandable members to anchor medical device 22000 within a lung airway.

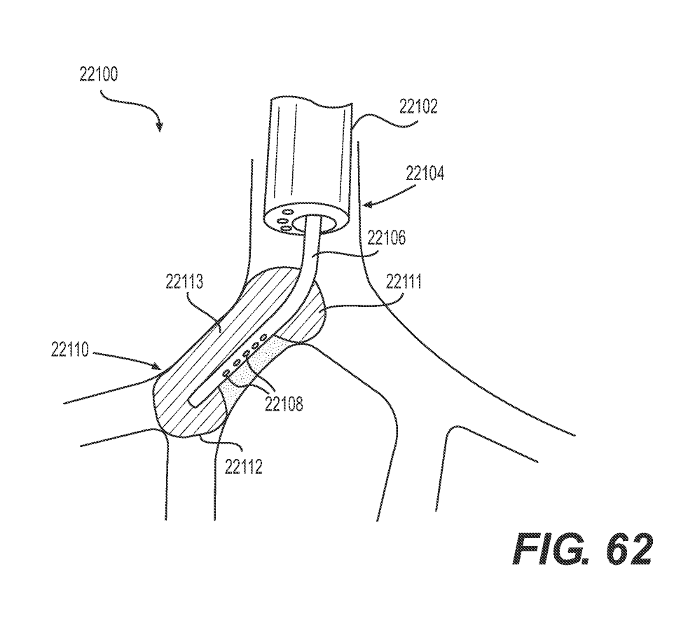

A medical device 22100 is shown in FIG. 62. An elongate member 22106 may extend from a distal end 22104 of a bronchoscopic member, e.g., a bronchoscope. Medical device 22100 may include one or more openings 22108 disposed along elongate member 22106 that are substantially similar to openings 21608 described with reference to FIG. 57. Each opening 22108 may be coupled to a source of cooled substance (not shown), and may be configured to deliver an amount of cryospray to airways of the lung.

An expandable member 22110 may be disposed proximally, distally, and partially circumferentially around openings 22108 along elongate member 22106. That is, expandable member 22110 may have a proximal portion 22111, a distal portion 22112, and a circumferential portion 22113 disposed adjacent to, and at the same axial location as openings 22108. Expandable member 22110 may be movable between a first, unexpanded/deflated configuration (not shown), and a second, expanded/inflated configuration (shown in FIG. 62). Expandable member 22110 may be a balloon or similar expandable member configured to create a seal around a partially-circumferential treatment region to allow an operator to achieve specificity of treatment along a portion of a lung airway for delivering cryospray. That is, expandable member 22110 may be configured to deliver cryospray to an isolated, radial portion of a lung airway. In one embodiment, all portions of expandable member 22110 may be in fluid communication with one another. In an alternative embodiment, separate portions of expandable member may be separately inflatable/expandable. In one embodiment, only proximal portion 22111 and circumferential portion 22113 may be expanded to permit some spray to go distally. In another embodiment, only proximal portion 22111 and distal portion 22112 may be expanded so that cryospray is delivered to the entire circumferential region between proximal portion 22111 and distal portion 22112. In yet another embodiment, all of proximal portion 22111, distal portion 22112, and circumferential portion 22113 may be expanded to isolate a portion of the circumference for cryospray delivery. Medical device 22100 may include one or more lumens (not shown) that are substantially similar to the lumens described with reference to FIG. 58. Thus, medical device 22100 may allow for pre- and post-treatment aspiration or cleaning of airway surfaces in the region sealed by expandable member 22110.

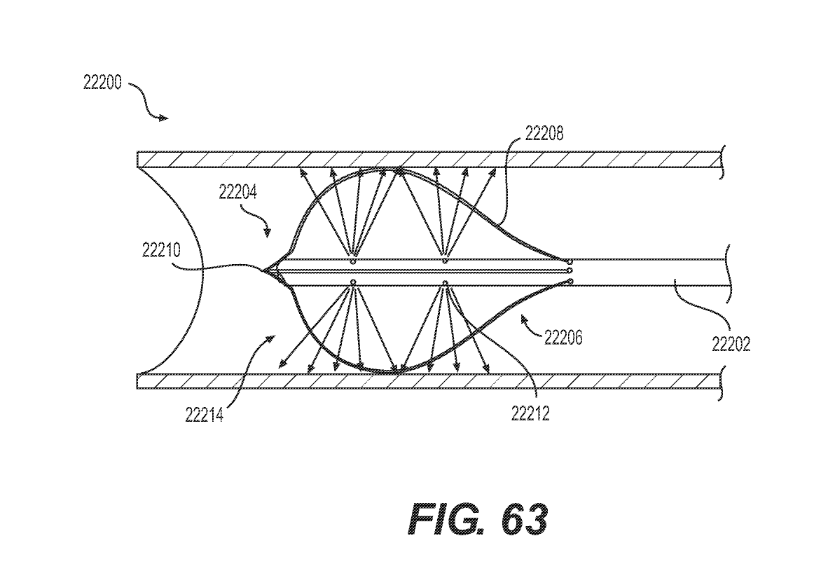

A medical device 22200 is shown in FIG. 63 disposed within a lung airway. Medical device 22200 may include an elongate member 22202 extending from a proximal end (not shown) toward a distal end 22204. An expandable distal member such as an expandable basket 22206 may be located at distal end 22204 of elongate member 22202. Expandable basket 22206 may be reciprocally movable between an expanded configuration (shown in FIG. 63) and a collapsed/retracted configuration (not shown). In some embodiments, expandable basket 22206 may instead be arranged as a nest, globe, or other suitable expandable member.

In some embodiments, expandable basket 22206 may include a plurality of legs 22208 configured to anchor distal end 22204 of elongate member 22202 within the lung airway. In the embodiment shown, expandable basket 22206 has four legs 22208 that are equally spaced. However, expandable basket 22206 may alternatively include any other suitable number of legs 22208 in any suitable spacing arrangement. In some embodiments, the legs 22208 may be coupled at a distal tip 22210 using any suitable technique such as, but not limited to soldering, welding, or the like. However, in other embodiments, the legs 22208 may not be connected at the distal tip 22210, and the expandable distal member may be formed as a prong or another suitable shape. In some embodiments, an arcuate surface of the legs 22208 may come in contact with the airway wall when the expandable basket 22206 is expanded radially. Actuation of expandable basket 22206 may be by any suitable mechanism, such as, e.g., a pull wire extending through elongate member 22202 that is coupled to the distal ends of legs 22208.

Medical device 22200 may include one or more openings 22212 disposed along elongate member 22202 that are substantially similar to openings 21608 described with reference to FIG. 57. Each opening 22212 may be coupled to a source of cooled substance (not shown), and may be configured to deliver an amount of cryospray 22214 to airways of the lung. Medical device 22200 may include an exhaust lumen (not shown) that is substantially similar to the exhaust lumen described with reference to FIG. 57.

Expandable basket 22206 may be formed of any suitable material including biocompatible metals, alloys, or other materials. In some embodiments, expandable basket 22206 may be formed from stainless steel, nitinol, aluminum, or the like. In some embodiments, one or more of legs 222208 may function as a temperature sensing element. Legs 2208 may also be coupled to a source of electrical energy, such as, e.g., RF energy to apply cryo and heating device to treat portions of lung airways.

It should be noted that the embodiments of FIGS. 57-63 may be utilized to deliver any suitable substance in addition or alternative to a cryospray, such as, e.g., a neurolytic agent. In some embodiments, a neurolytic agent may be delivered in addition to cryospray to further damage nerves of the lung.

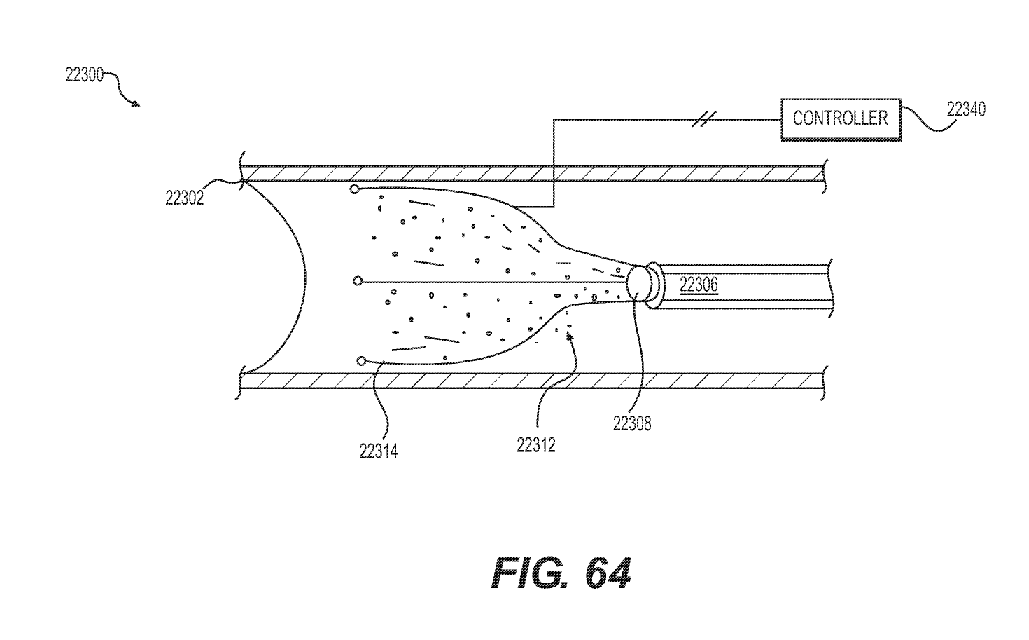

A medical device 22300 is shown in FIG. 64. Medical device 22300 may include an elongate member 22306, an opening 22308, a plurality of sensing elements 22314, and a controller 22340.

The elongate member 22306 may be a flexible, hollow member that is substantially similar to elongate member 21606 described with reference to FIG. 57. Opening 22308 may be substantially similar to opening 21808 described with reference to FIGS. 59-61. In some embodiments, elongate member 22306 may be formed of a radiopaque or other imaging material, or have suitable imaging markings, so that it can be visualized under fluoroscopic guidance. The radiopaque or other imaging materials may be used to ensure that a correct location of elongate member 22306 and opening 22308 are achieved. In some embodiments, opening 22308 may be prevented from activating by controller 22340 until the radiopaque or imaging material is appropriately positioned.

The medical device 22300 may be configured to deliver a cryospray 22312 in a distal direction through opening 22308, in a substantially similar manner as cryospray 21809 described with reference to FIGS. 59-61. Cryospray 22312 may be substantially similar to cryospray 21809 described with reference to FIGS. 59-61.

The volume and speed of the delivered cryospray 22312 may be dictated by the shape of opening 22308, among other factors. In one embodiment, opening 22308 may be narrow (e.g., a nozzle) for atomization of the cryospray 22312. Similarly, various shapes and dimensions of opening 22308 may be used to provide different spray patterns, varying atomization rates, and varying the direction of the emergent cryospray 22312. Medical device 22300 may include an exhaust lumen (not shown) that is substantially similar to the exhaust lumen described with reference to FIG. 57.

In some embodiments, medical device 22300 may include multiple openings 22308 that are substantially similar to openings 21608 described with reference to FIG. 57. In such embodiments, openings 22308 may be configured to deliver cryospray 22312 in a proximal direction, a distal direction, or in a direction that is substantially orthogonal to the longitudinal axis of elongate member 22306. In some embodiments, gas-assisted atomization may be performed. In such embodiments, the elongate member 22306 may include two openings such that a first opening may be configured to inject a liquid, and a second opening may inject a gas pressurized for gas-assisted atomization. The resulting high shear forces on the liquid may cause the atomization of the liquid to deliver a fine cryospray 22312. In some embodiments, the gas opening may be concentric with the liquid opening.

The medical device 22300 may further employ a temperature sensing and/or feedback mechanism to measure the temperature of a lung airway wall. Such a mechanism may help avoid causing an excessive cooling of the lung airways to optimize the delivery of cryospray 22312 to the lungs. To accomplish this, a plurality of sensing elements 22314 may extend radially outwards from a distal portion of the elongate member 22306 conforming to a lung airway wall 22302. Alternatively or additionally, the temperature sensing elements 22314 may be configured to penetrate into the airway wall, e.g. to a depth between 0.2 to 2 mm, or another suitable depth, to measure temperature at a distance beyond the airway surface. The penetration of temperature sensing elements beyond the airway surface may help give a better indication of temperature at airway smooth muscle or at nerve trunks near the exterior surface of the airway wall. Temperature sensing elements 22314 may be utilized in any disclosed embodiment utilizing a temperature changing modality to damage nerves, such as, e.g., RF, HIFU, microwave, cryo, or other suitable modalities.

Each sensing element 22314 may be formed as a prong configured to be in close proximity to (e.g., in contact with) the airway wall 22302 to sense the temperature of the lung airway. The sensed temperature of the lung airway may be indicative of the temperature of nerves disposed within the lung airway or beyond the lung airway. To accomplish this, the plurality of sensing elements 22314 may be configured to move between a retracted configuration within elongate member 22306 (not shown) and an expanded configuration (shown in FIG. 64). In the expanded configuration, the sensing elements 22314 may expand radially outwards to form an umbrella-shaped configuration. Additionally, the plurality of sensing elements 22314 may contact the airway wall 22302 in the expanded position to sense the temperature of the lung airway. In some embodiments, the sensing elements 22314 can be expanded once the medical device 22300 is deployed into the lung airway, if desired. The temperatures sensed by sensing elements 22314 may be indicative of a temperature of lung airway nerves such as afferent sensory nerves, efferent nerves, or the like.

In the illustrated embodiment, three sensing elements 22314 are depicted, although any suitable number of sensing elements 22314 may be utilized. In one embodiment, the sensing elements 22314 may be arranged substantially parallel to a longitudinal axis of elongate member 22306 and equally spaced radially about the longitudinal axis. In other embodiments, the sensing elements 22314 may be arranged in another suitable arrangement. In some embodiments, the sensing elements 22314 may be placed at a uniform distance from one another, or may be alternatively arranged at a varying distance from one another. In addition, the length of each sensing element 22314 may either be same, or may vary from one another such that different sensing elements 22314 can be positioned at different regions within the lung airways. In some embodiments, there may each prong or sensing elements 22314 may include multiple sensing elements on the same prong. The one or more sensing elements 22314 may form a matrix of sensors from which the gathered temperature data may be interpolated using a software algorithm to form a complete thermal map of the inside of the airway. This data may be combined with time to form a temperature-time exposure map of the airway tissue to facilitate the determination and control of the effect of cryospray 22312 on the airway tissue.

In one embodiment, the sensing elements 22314 may include temperature sensing probes such as thermocouples, thermistors, IR sensors, or the like.

Controller 22340 may be operably coupled to the plurality of sensing elements 22314, and may be configured to control the output of cryospray 22312. Alternatively, the output of cryospray 22312 may be controlled manually by an operator. Controller 22340 may be coupled to a user interface (not shown) that may include switches, a digital display, visual indicators, audio, as well as other features. Controller 22340 may include a processor that is generally configured to accept information about sensing elements 22314 and other components, and process the received information according to various algorithms to produce control signals for controlling cryospray 22312. The processor may be digital IC processor, analog processor or any other suitable logic or control system that carries out the control algorithms. In one embodiment, controller 22340 may be configured to dispense cryospray 22312 based on a temperature feedback loop utilizing inputs from sensing elements 22314. The temperature feedback loop may be any suitable loop such as, e.g., a proportional-integral-derivative (PID) loop, among others, that allows controller 22340 to send appropriate instructions to dispense cryospray 22312. The feedback loop may allow controller 22340 to apply cryospray 22312 to lower the temperature of the lung airways based on a temperature sensed by sensing elements 22314.

Thus, controller 22340 may be configured to use inputs from sensing elements 22314 in a temperature feedback loop to control an output of cryospray 22312. In some embodiments, the input may include the temperature of the lung airways at one or more locations, and the output may include one or more deployment parameter(s) for dispensing cryospray 22312. In some embodiments, the deployment parameter may include a proximal speed of elongate member 22306 during application of cryospray 22312 (i.e., the speed at which elongate member 22306 is retracted proximally). The proximal speed of the elongate member 22306 (or distal speed) may determine the time period to which an area of the lung airway may be subjected to the cryospray application, and therefore control the temperature of lung airways and/or nerves within or beyond airway wall 22302. In some embodiments, elongate member 22306 may be actuated by one or more pull lines, or other suitable mechanisms, that steer elongate member 22306. This temperature sensing functionality also may be useful when elongate member 22306 is positioned and deployed manually, as it may enable an operator to determine the extent and duration of the cooling therapy applied.

According to one embodiment, if the temperature of the lung airways is below or approaching a treatment threshold temperature (e.g., approaching or below −20° C.), the proximal speed of the elongate member 22306 may be increased during application of cryospray 22312. That is, by increasing the speed that elongate member 22306 travels through the lung airway, less cryospray 22312 may be delivered to a particular region of the airway, preventing damage to the lung airway as a result of applying too much cryospray 22312 in the particular region. Alternatively, if the temperature of the lung airway is above the treatment threshold temperature (e.g., above −5° C.), the proximal speed of the elongate member 22306 may be decreased. That is, by decreasing the speed that elongate member 22306 travels through the lung airway, an additional amount of cryospray 22312 may be delivered to a particular region of the airway to lessen the time for which a certain region is treated.

In the above embodiment, the proximal (or distal) speed of the elongate member 22306 may be used as a deployment parameter. However, other suitable deployment parameters, for example, a volume or pressure of cryospray 22312 that is applied, are also contemplated. Thus, if controller 22340 determines that the temperature of airway wall 22302 is below or approaching a treatment threshold temperature, controller 22340 may decrease the volume or pressure of cryospray 22312 applied to the lung airways. On the contrary, if controller 22340 determines that the temperature of airway wall 22302 is above a treatment threshold temperature, controller 22340 may increase the volume or pressure of cryospray 22312 applied to the lung airways.

It should be noted that temperature sensing elements 22314 and controller 22340 may be used in conjunction with any embodiment of the present disclosure, including embodiments utilizing cryospray and embodiments not utilizing cryospray. Thus, temperature sensing elements 22314 also may be utilized in configurations where cooling therapy is delivered by cooling members, such as, e.g., balloons, cryoprobes, lumens, or the like.

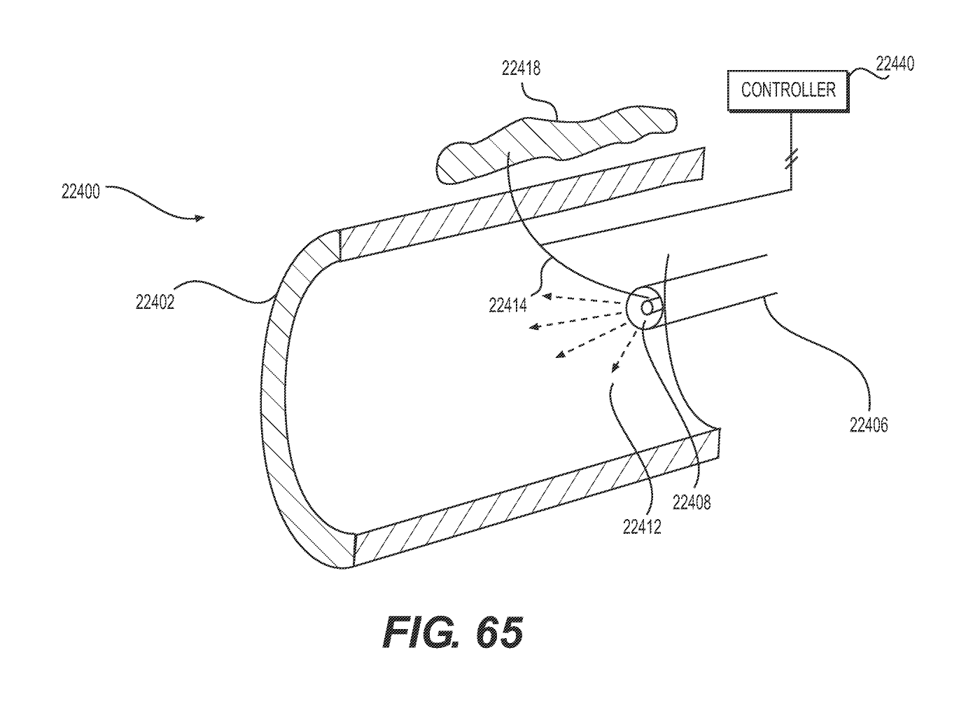

Referring now to FIG. 65, a medical device 22400 inserted into a lung airway having an airway wall 22402 is depicted. In FIG. 65, the medical device 22400 may include an elongate member 22406, an opening 22408, and a controller 22440. The opening 22408 may be configured to deliver a cryospray 22412 that is delivered in a substantially similar manner that cryospray 22312 is delivered with reference to FIG. 64. Elongate member 22406, opening 22408, and cryospray 22412 may be substantially similar to elongate member 22306, opening 22308, and cryospray 22312 of FIG. 64. Medical device 22400 may include an exhaust lumen (not shown) that is substantially similar to the exhaust lumen described with reference to FIG. 57.

The medical device 22400 may further include a sensing element 22414 configured to sense the temperature of a nerve 22418, which may be disposed further away from the elongate member 22406 than the inner surface of airway wall 22402. The nerve 22418 may be an afferent sensory nerve, efferent nerve, or another suitable nerve.

Sensing element 22414 may be formed as a prong or another suitable member. Although a single sensing element 22414 is shown in the embodiment of FIG. 65, it should be contemplated that any suitable number of sensing elements 22414 may be employed to accomplish the desired purposes. In addition, the sensing element 22414 may be configured to move between a retracted configuration within elongate member 22406 (not shown) and an expanded configuration (shown in FIG. 65). In the expanded configuration, the sensing element 22414 may expand distally and radially outwards (e.g., deflect) to come in close proximity or direct contact with to the nerve 22418 to directly sense a temperature of nerve 22418. In some embodiments, as sensing element 22414 expands radially outward/deflect, sensing element 22414 may pierce or otherwise pass through airway wall 22402 to be placed in contact with nerve 22418. In some embodiments, the distal end of sensing element 22414 may be beveled or otherwise sufficiently sharp to pierce tissue. In one embodiment, sensing element 22414 may be pre-curved or bent radially outward such that sensing element 22414 pierces through airway wall 22402 when moved distally from elongate member 22406. In some embodiments, sensing element 22414 may be selectively steerable via an actuator (not shown) at the proximal end of medical device 22400. Because sensing element 22414 may be in close proximity or in direct contact with nerve 22418, a more accurate temperature of nerve 22418 may be sensed. Sensing element 22414 may be otherwise substantially similar in structure to sensing element 22314 described with reference to FIG. 64. Temperature sensing elements 22414 may be utilized in any disclosed embodiment utilizing a temperature changing modality to damage nerves, such as, e.g., RF, HIFU, microwave, cryo, or other suitable modalities.

The medical device 22400 may include a controller 22440 that may be operably coupled to the sensing element 22414. Controller 22440 may be substantially similar to controller 22340 described with reference to FIG. 64. Controller 22440 may thus help control a temperature of lung airways and nerves so that they fall within a desired treatment threshold temperature (e.g., −5° C. to −20° C.) based upon a measured temperature input received from the sensing elements 22414. The controller 22440 then may provide an output or control signal to deliver cryospray 22412 through opening 22408. As a result, a deployment parameter, such as a proximal (or distal) speed of the elongate member 22406 may be varied to control the quantity of the cryospray 22412 injected adjacent the target nerve 22418. It should be noted that the cryospray 22412 may be applied and/or delivered simultaneously with the proximal movement of the elongate member 22406 to reduce a temperature of the treatment location (e.g., the lung airways). Other parameters, such as a volume or pressure of cryospray 2412, among others, may also be controlled by controller 22440 in a substantially similar manner to controller 22340 described with reference to FIG. 64.

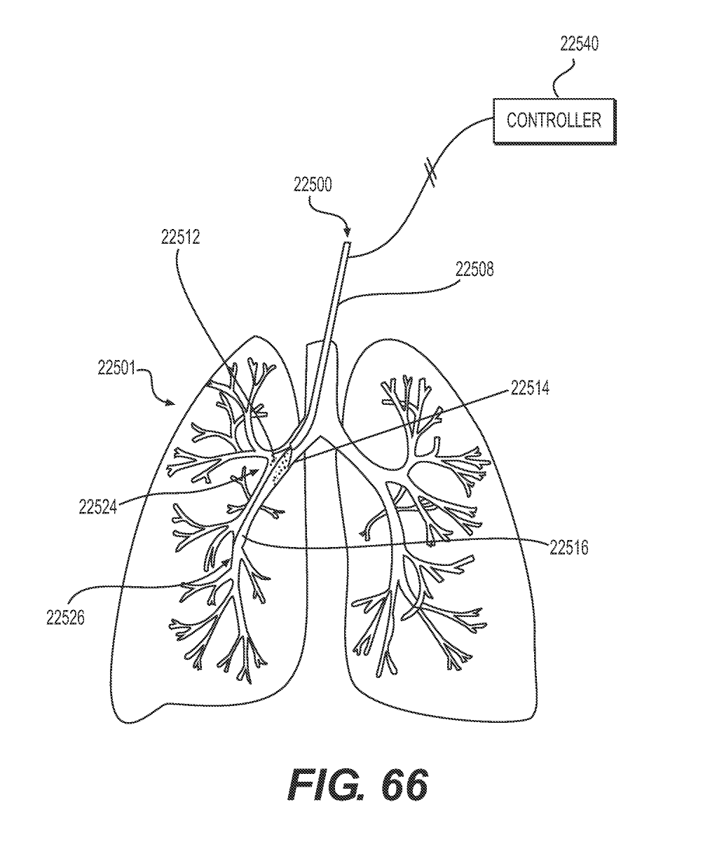

FIG. 66 is an in vivo illustration of a medical device 22500 disposed within a lung system 22501, according to one embodiment of the present disclosure. The medical device 22500 may include an elongate member 22508 configured to be introduced into a proximal or upstream airway 22524 of lung system 22501. Proximal airway 22524 may be a bronchus, bronchiole, or another airway disposed within lung system 22501. The medical device 22500 may include a proximal sensing element 22514 located adjacent or within the proximal airway 22524 and a distal sensing element 22516 located adjacent or within a distal or downstream airway 22526. Distal airway 22526 may be a bronchus, bronchiole, or other airway disposed within a lung that is distal or downstream of proximal airway 22524. Medical device 22500 may additionally include any other suitable number of sensing elements. In the embodiment of FIG. 66, the two sensing elements 22514, 22516 may be configured to sense the temperature of lung system 22501 at the two airways 22524, 22526. It should be noted that the elongate member 22508 and the sensing elements 22514, 22516 may have similar form and function to that of the elongate members 2306, 2406 and sensing elements 22314, 22414 of FIGS. 64 and 65. Medical device 22500 may include an exhaust lumen (not shown) that is substantially similar to the exhaust lumen described with reference to FIG. 57.

In some embodiments, cryospray 22512 may be applied to the proximal airway 22524 of a patient. While cryospray 22512 is applied to the proximal airway 22524, distal sensing element 22516 may be disposed at distal airway 22526. Thus, the distal sensing element 22516 may be configured to sense the temperature at an airway that is distal or downstream of where cryospray 22512 is applied. In such embodiments, the distance between the distal sensing element 22516 and the emergent cryospray 22512 may be sufficient enough to avoid any contact between cryospray 22512 and distal sensing element 22516.

As cryospray 22512 is applied to the proximal airway 22524 to lower the temperature of proximal airway 22524, a temperature may be sensed at one or both of proximal airway 22524 and distal airway 22526 and provided as an inputs to a controller 22540. The temperature sensed at proximal airway 22524 and/or distal airway 22526 may be performed in a manner substantially similar to those described with reference to FIGS. 64 and 65. The controller 22540 may be configured to alter a deployment parameter of the cryospray 22512 based on the sensed temperatures. The deployment parameter may include the proximal (or distal) speed of elongate member 22508 that may be varied to inject a particular amount of the cryospray 22512 within the airway 22501. Other parameters, such as a volume or pressure of cryospray 22512, among others, may also be controlled by controller 22540 in a substantially similar manner to controllers 22340 and 22440 described with reference to FIGS. 64 and 65.

Further, distal airway 22526 may be an airway that is not intended to be treated with cryospray 22512. Thus, controller 22540 may determine, based on inputs received from distal sensing element 22516, that tissue regions unintended for cryospray treatment are being cooled to inappropriate levels. Thus, if distal element 22516 senses that distal airway 22526 is approaching, or has reached a threshold temperature for untreated tissue (e.g., 0-37° C., or another suitable temperature to prevent bronchospasms or other physiologic effects), controller 22540 may take measures to prevent further cooling of distal airway 22526. It should be noted that proximal and distal temperature sensing elements 22514 and 22516 may be utilized in applications that heat lung airways. In such embodiments, if distal element 22516 senses that distal airway 22526 is approaching, or has reached, a high threshold temperature for untreated tissue, controller 22540 may similarly take measures to prevent further heating of distal airway 22526. The measures may include preventing further application of cryospray 22512, adjusting a position of medical device 22500, sending a warning to an operator of medical device 22500, among others. Alternatively, distal sensing element 22516 may be deployed in other suitable locations to help prevent undesired damage to surrounding tissue. For example, distal sensing element 22516 may be disposed in an airway that is proximal and/or adjacent proximal airway 22524. The proximal and/or adjacent airway may not be designated to receive an application of cryospray 22512. Thus, controller 22540 may monitor sensing element 22516 to help prevent the inadvertent cooling and damage of non-targeted airways.

It should be noted that the sensing elements 22514, 22516, the cryospray 22512, and the controller 22540 may have similar form and function to that of the sensing elements 22314, 22414, cryosprays 22312, 22412, and controllers 22340, 22440 described with reference to FIGS. 64 and 65.

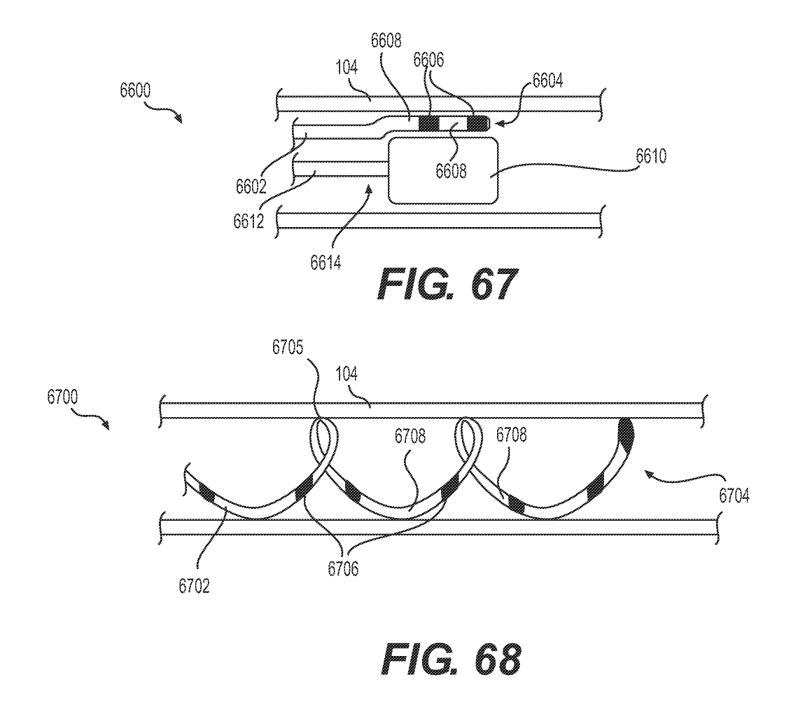

A medical device 6600 is shown in FIG. 67. Medical device 6600 may include an elongate member 6602 extending from a proximal end (not shown) toward a distal end 6604. Elongate member 6602 may be configured to advance through another elongate member, such as, e.g., a bronchoscope. Elongate member 6602 may include one or more active regions 6606 and insulated regions 6608 that are substantially similar to active regions 21208 and insulated regions 21210 described with reference to FIG. 53. Medical device 6600 may include a second elongate member 6612 that is also configured to extend from a bronchoscope. The second elongate member 6612 may include an expandable member 6610 at a distal end 6614. The expandable member 6610 may, in an expanded configuration, position one or more active regions 6606 of elongate member 6602 against or otherwise in contact with the surface of epithelial tissue 104. In one embodiment, expandable member 6610 may position a first set of active regions 6606 against epithelial tissue 104, and may be moved longitudinally (in a collapsed or expanded configuration) along epithelial tissue 104, to position subsequent sets of active regions 6606 against epithelial tissue 104. Elongate members 6602 and 6612 may be independently actuatable and translatable, or may be actuated and translated together, if desired.

A medical device 6700 is shown in FIG. 68. Medical device 6700 may include an elongate member 6702 extending from a proximal end (not shown) toward a distal end 6704. Elongate member 6702 may be configured to advance through another elongate member, such as, e.g., a bronchoscope. Elongate member 6702 may include one or more active regions 6706 and insulated regions 6708 that are substantially similar to active regions 21208 and insulated regions 21210 described with reference to FIG. 53. Elongate member 6702 may have a spiral, wavy, sinusoidal, or other similar configuration such that elongate member 6702 has one or more coils 6705. In some embodiments, elongate member 6702 may be substantially linear when constrained within a bronchoscope, but may assume the spiral, wavy, or sinusoidal shape after exiting the bronchoscope. Medical device 6700 may be configured to deliver a spiral treatment to epithelial tissue 104, maintaining mucociliary function, and may be adaptable to any dimensioned airway (e.g., smaller and larger airways alike).

High-Intensity Ultrasound (HIFU)

In some embodiments, HIFU may be applied to a lung airway in a controlled manner to damage afferent sensory nerves, efferent nerves, or the like. The HIFU may be configured to damage or destroy nerve function, including the ability of a targeted nerve to transmit signals.

Delivery of the HIFU may be through the right main bronchus, left main bronchus, or both, as treating only one of the right or left main bronchi may be sufficient for a significant reduction in bronchoconstriction and/or mucus production, as the right and left vagus nerves traverse along the right and left main bronchi, respectively. In some embodiments, HIFU may be applied to the four second generation bronchi, or the eight third generation bronchi, for example. The benefit of treating further down (distally) in the lung is that the airway wall is thinner and target nerves may be located (relatively) closer to the energy delivery system so that less energy may be required to effectively treat the targeted nerve.

Further, once inside the parenchyma (inside the lung, past the hilum), the parenchyma may serve as an energy barrier for HIFU energy. HIFU energy does not easily transmit through the air, and thus, in the second, third, or distal airways, the parenchyma (which is filled with air) may serve as a barrier to HIFU energy. This barrier may reduce the likelihood of adverse effects beyond the target treatment zone (e.g., targeted nerve). As the parenchyma does not surround the first generation airways, this benefit may not be present in HIFU treatments of the first generation airways.

Linking the delivery of HIFU (or any other energy modality) with a detection system such as, e.g., an electrode mapping catheter, to a localized treatment location may allow for a more specific treatment to be applied, potentially reducing the damage to adjacent tissues. Other imaging procedures, such as, e.g., magnetic resonance imaging (MRI), diagnostic sonography, or other suitable imaging techniques also may be used.

In some embodiments, the HIFU may locally heat and ablate a targeted area. In some embodiments, the acoustic energy delivered via HIFU may have a frequency in the range of 20 kHz to 20 MHz, although other suitable frequencies are also contemplated. In some embodiments, the acoustic energy may be configured to ablate tissue at a certain depth (e.g., 1 mm), while having a therapeutically negligible effect to tissue at a surface of the lung airways. Thus, in some embodiments, the HIFU may be configured to preserve mucociliary function of the lung airways by leaving the lung airway surfaces intact.

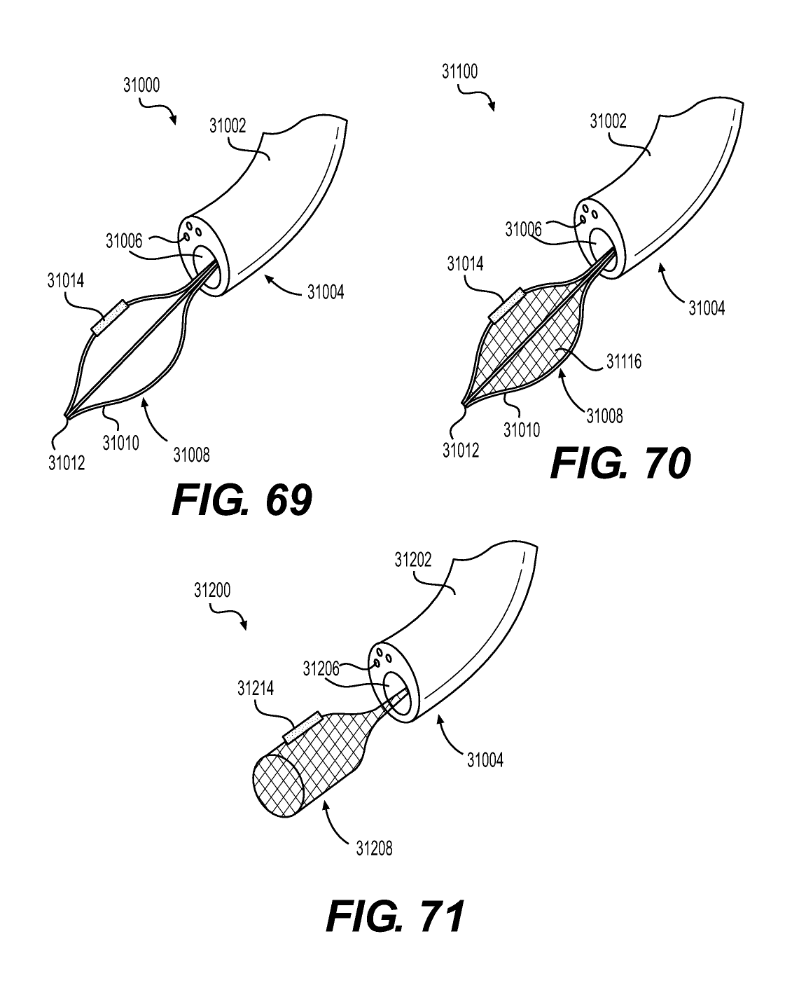

An energy delivery element 31014 (referring to FIG. 69) may be configured to deliver acoustic or sonic energy, such as, e.g., HIFU, to locally ablate a lung airway tissue or lung airway nerves. Delivery of HIFU may be via a minimally-invasive procedure that can ablate tissue located at a depth below the lung airway surface. The depth of energy delivery may be tuned, electronically adjusted, and/or mechanically adjusted. In some embodiments, collateral damage in certain airways may be of less concern due to the energy blocking effect of the parenchyma as described above. A HIFU beam may be directed to about 1.0 to 2.0 mm into the wall thickness, or to another suitable depth. If the HIFU treatment is used in conjunction with an imaging system (e.g. endobronchial ultrasound), then the target depth may be calculated during the procedure. For example, a depth measurement may be taken by a diagnostic ultrasound component, a calculation may be performed using the depth measurement to determine wall thickness, and then HIFU energy may be delivered to the airway. The diagnostic ultrasound component may be able to assess whether the target was actually treated after the application of HIFU energy.

In some embodiments, energy delivery element 31014 may not ablate tissue that is closest in proximity to energy delivery element 31014, but may instead only ablate tissue that is a predetermined distance from energy delivery element 31014. For example, energy delivery element 31014 may not ablate a closest 5 mm of tissue, while ablating tissue that is located between about 5 mm and 15 mm from energy delivery element 31014, if desired. In one embodiment, energy delivery element may not ablate a closest 0.2 mm of tissue, while ablating tissue that is located about 0.2 mm and 2 mm from energy delivery element 31014. In some embodiments, energy delivery element 31014 may not ablate a closest 1.0 mm of tissue, while ablating tissue that is located between about 1.0 mm and 3.0 mm from energy delivery element 31014. It should be noted that other suitable ablation ranges may alternatively be utilized. In some embodiments, energy delivery element 31014 may ablate tissue at a depth beyond the surface of a lung airway wall so as not to irreversibly damage the epithelial layer. The preservation of the epithelial layer may avoid unnecessary trauma, inflammation, and/or mucus production, while maintaining the mucociliary clearance functions of the lung airways being treated.

Energy delivery element 31014 may include a lens, curved transducer, phased array (e.g., linear phased array, curvilinear phased array, or convex sector phased array), or a combination thereof, configured to focus the ultrasound into a small focal zone. The curvature of the lens or transducer, for example, may direct the HIFU to the desired tissue depth. Multiples lenses, transducers, or other energy delivery elements may be used, where each such element has a structure for delivering energy at a different or same depth. If a different depth, one or more of the energy delivery elements may be selected according to the desired depth of treatment. In addition, the heating of target tissue may be proportional to the intensity of the ultrasound or energy applied, which may be inversely proportional to the area over which the ultrasound or energy is applied. The extent of tissue damage may be modeled using Cumulative Equivalent Minutes and/or other suitable formulas, which may be used by, e.g., controller 32 to select and control the amount of HIFU energy delivered.

In some embodiments, a location of the nerve(s) to be targeted in the airways may be determined by direct visualization, of, e.g., an anatomical structure, by ultrasound scanning/imaging, or by any other suitable means. Once a targeted nerve or treatment location is determined, energy delivery element 31014 may deliver HIFU energy to the targeted nerve or treatment location. In one embodiment, the targeted nerve or treatment location may be first detected by ultrasound scanning/imaging, and then HIFU energy may be delivered to a less than 360 degree circumference, e.g., a less than 90 degree circumference of the airway which corresponds with the target nerves or treatment location. In other embodiments, HIFU may be applied to an entire 360 degree circumference of the airway (e.g., in spiral treatment patterns). In some embodiments, little or no damage will be caused to the remaining circumference of the airways that are not targeted by energy delivery element 31014. In some embodiments, energy delivery element 31014 may be capable of both imaging and delivering a therapy. Alternatively, energy delivery element 31014 may be configured for energy delivery around a larger circumference of the airway or esophagus, and may be directed at additional locations other than nerve tissue. In some embodiments, energy delivery element 31014 may be directed toward smooth muscle tissue in the lung airways to achieve reduced bronchoconstriction (by e.g., scarring the smooth muscle tissue). In some embodiments, energy delivery element 31014 may be directed toward tissues and body elements affecting other diseases such as, e.g., asthma, chronic cough, chronic bronchitis, and Cystic Fibrosis, where bronchoconstriction, mucus hypersecretion, and cough are also observed. HIFU may be utilized in tumor ablation or lung cancer treatments where HIFU may be coupled to the tumor via solution (e.g., saline, or another liquid or gel).

Delivery Devices

A medical device 31000 shown in FIG. 69 may include an bronchoscopic member 31002, such as, e.g., a bronchoscope or guide catheter, extending from a proximal end (not shown, generally outside of a patient) toward a distal end 31004. A plurality of channels 31006 may be disposed through bronchoscopic member 31002 to allow for a variety of tools or fluids to be passed through bronchoscopic member 31002. One such tool includes an expandable distal member, such as a basket 31008, extending distally from distal end 31004 of bronchoscopic member 31002. The tool translates within the corresponding lumen of member 31002. Basket 31008 may include a plurality of legs 1010 movable between an expanded configuration (shown in FIG. 69) and a retracted configuration (not shown). The plurality of legs 31010 may be coupled at a distal tip 31012, which may be atraumatic. Basket 31008 may be actuated by any suitable mechanism, such as, e.g., a pull wire extending through bronchoscopic member 31002 and basket 31008 toward distal tip 31012. Alternatively, basket 31008 may be self-expandable and may be urged toward the expanded configuration as basket 31008 is deployed distally from bronchoscopic member 31002. Basket 31008 may be formed from any suitable material, such as, e.g., Nitinol, stainless steel, Elgiloy, polyurethane (PU), polytetrafluoroethylene (PTFE), ethylene tetrafluoroethylene (ETFE), perfluoroalkoxy (PFA), polyether ether ketone (PEEK), high density polyethylene (HDPE), polypropylene (PP), or other suitable materials.

At least one energy delivery element 31014 may be disposed on at least one of legs 31010. In the embodiment shown in FIG. 69, basket 31008 has four legs 31010 (one of the four legs 31010 having an energy delivery element 31014 disposed thereon), though any other suitable number of legs 31010, their spacing relative to each other, and energy delivery elements 31014, may be utilized. That is, medical device 31000 may include a plurality of energy delivery elements 31014 to deliver energy to multiple treatment locations simultaneously. In some embodiments, the legs 31010 that do not contain an energy delivery element 31014 may expand outwardly against a lung airway wall to anchor medical device 31000 within the lung airway. Legs 31010 of basket 31008 may extend distally from distal end 31004 of bronchoscopic member 31002 via an elongate member (not shown), such as a sheath, catheter, or another suitable elongate member that receives the proximal ends of legs 31010. In an alternative embodiment, energy delivery element 31014 may be disposed within basket 31008. In such embodiments, basket 31008 may act as an anchor for energy delivery element 31014 within a lung airway.

Legs 31010 may be formed of any biocompatible material. In some embodiments, legs 31010 may be self-expanding, and formed from any flexible elastic or superelastic biocompatible material. In some embodiments, legs 31010 may be formed from one or more of nitinol, stainless steel, Elgiloy, or other suitable biocompatible materials. Legs 31010 may have a smooth lubricious coating and/or have an atraumatic configuration. However, it should be noted, that in some embodiments, the outer surface of legs 31010 may maintain a sufficient frictional force with the airway wall so that medical device 31000 does not move with normal breathing. Thus, in some embodiments, the outer regions of legs 31010 may have a roughened surface (e.g., a plurality of protrusions) to increase frictional force between legs 31010 and the lung airways. In some embodiments, legs 31010 may include a material or webbing spanning between two or more legs 31010 that may be configured to further shield certain tissues from HIFU energy. Legs 31010 also may be formed of a radiopaque material so that they can be visualized under fluoroscopic guidance, or energy delivery element 31014 may otherwise include radiopaque or other imaging markers for guidance. The markers may be used to ensure that a correct direction of therapy is applied. In some embodiments, legs 31010 and energy delivery element 31014 may be prevented from activating until the marker is appropriately positioned.

When basket 31008 is in the expanded configuration, energy delivery element 31014 may be urged toward the surface of a lung airway, improving the transmission of energy from energy delivery element 31014 to a targeted treatment location.

In some embodiments, medical device 31000 may include one or more sensors to detect various parameters or anatomical structures. In one embodiment, the one or more sensors may include temperature sensors configured to detect a presence/amount of therapy delivered. In another embodiment, the one or more sensors may include structures within the lung airway configured to use Doppler ultrasound to detect blood vessels. In another embodiment, the one or more sensors may sense electrical measurement of nerve traffic that corresponds to an efficacy of the treatment. In another embodiment, the one or more sensors may include a vision system for direct observation. In yet another embodiment, the one or more sensors may include a force transducer, strain gauge, or similar sensor to measure radial force in the lung airways. The one or more sensors may also be combined with any of medical devices, delivery devices, or elongate members described by the present disclosure. In yet another embodiment, one or more sensors may generate an impedance measurement that detects changes in tissue properties.

Referring to FIG. 70, medical device 31100 may be substantially similar to medical device 31000 described with reference to FIG. 69, except that a mesh 31116 may be disposed between adjacent legs 31010 of medical device 31100. Mesh 31116 may be formed of the same material or of a different material than legs 1010. In some embodiments, mesh 31116 may be a webbing configured to shield certain tissues from receiving HIFU energy. Mesh 1116 also may extend across an interior of basket 1008 by attaching to opposing legs 1010. Mesh 31116 may provide better geometric control of legs 31010 with respect to one another. That is, mesh 31116 may provide a more predictable spacing between legs 31010, which may allow for more accurate positioning of one or more energy delivery elements 31014.

Referring to FIG. 71, a medical device 31200 may include an bronchoscopic member 31202 and channels 31206 that are substantially similar to bronchoscopic member 31002 and channels 31006 described with reference to FIG. 69. An expandable distal member 31208 may extend distally from a distal end 31204 of bronchoscopic member 31202. Expandable distal member 31208 may be movable between an expanded configuration (shown in FIG. 71) and a retracted configuration (not shown). In some embodiments, expandable distal member 31208 may be self-expandable. That is, expandable distal member 31208 may be biased, pre-bent, or have a shape-memory in the expanded configuration such that expandable distal member 31208 is urged radially outward as it is pushed from distal end 31204 of bronchoscopic member 31202.

At least one energy delivery element 31214 may be disposed on a surface of expandable distal member 31208. Energy delivery element 31214 may be substantially similar to energy delivery element 31014 described with reference to FIG. 69. Medical device 31200 may include a plurality of energy delivery elements 31214 to deliver energy to multiple treatment locations simultaneously. For example, elements 31214 can be spaced circumferentially about member 31208 (for circumferential treatment), axially spaced along member 31208 (for axial treatment), or both for spiral treatment, for example. All or a portion of medical device 31200 may be formed of a radiopaque material so that it can be visualized under fluoroscopic guidance, or energy delivery element 31214 may otherwise include radiopaque or other imaging markers for guidance

In the embodiment shown in FIG. 71, expandable distal member 31208 is a cylindrical stent, although any other suitable shape or structure may alternatively be utilized. Expandable distal member 31208 may expand outwardly against a lung airway wall to anchor medical device 31200 within the lung airway. Expandable distal member 31208 may extend distally from distal end 31204 of bronchoscopic member 31202 via an elongate member (not shown), such as a sheath, catheter, or another suitable elongate member that receives the proximal end of expandable distal member 31208. In an alternative embodiment, energy delivery element 31214 may be disposed within expandable distal member 31208. In such embodiments, expandable distal member 31208 may act as an anchor for energy delivery element 31214 within a lung airway.

Expandable distal member 31208 may be formed of substantially the same materials as legs 31010 described with reference to FIG. 69, and may have a smooth lubricous coating and/or have an atraumatic configuration. In some embodiments, expandable distal member 31208 may include a material or webbing that may be configured to further shield certain tissues from HIFU energy. Such webbing can extend around and along all or a portion of member 31208. If about only a portion, then the webbing can shield energy from desired locations relative to element 31214. Expandable distal member 31208 and/or energy delivery element 31214 also may be formed of a radiopaque material so that they can be visualized under fluoroscopic guidance in a substantially similar manner as described with reference to basket 31008 and energy delivery element 31014.

When expandable distal member 31208 is in the expanded configuration, energy delivery element 31214 may be urged toward the surface of a lung airway, improving the transmission of energy from energy delivery element 31214 to a targeted treatment location.

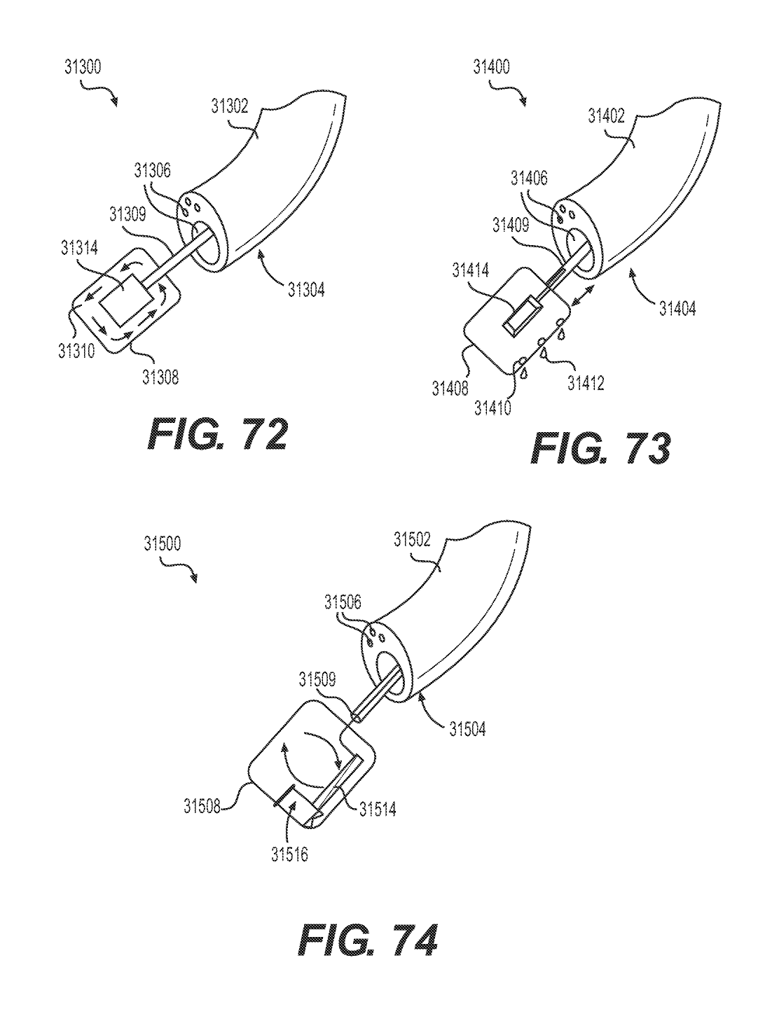

Referring to FIG. 72, a medical device 31300 may include an bronchoscopic member 31302 and channels 31306 that are substantially similar to bronchoscopic member 31002 and channels 31006 described with reference to FIG. 69. An expandable or inflatable member 31308, such as, e.g., a balloon, may extend distally from a distal end 31304 of bronchoscopic member 31302 via an elongate member 31309, such as, e.g., a sheath, catheter, or other suitable elongate member. Inflatable member 31308 may be movable between an inflated/expanded configuration (shown in FIG. 72) and a deflated/collapsed configuration (not shown). In the deflated/collapsed configuration, inflatable member 31308 may occupy a smaller volume than when in the inflated configuration, and an outer surface of inflatable member 31308 may not contact a lung airway wall. In the deflated/collapsed configuration, inflatable member 31308 may be delivered through bronchoscopic member 31302 to a treatment site. Inflation of member 31308 may be via a fluid delivered through a lumen of elongate member 31309.

At least one energy delivery element 31314 may be disposed within inflatable member 31308. Energy delivery element 31314 may be substantially similar to energy delivery element 31014 described with reference to FIG. 69.

Medical device 31300, when inflatable member 31308 is in the inflated/expanded configuration, may include a fluid 31310 disposed within inflatable member 31308 that may couple energy delivery element 31314 to inflatable member 31308, improving the transmission of energy between energy delivery element 31314 and the lung airway walls. In the inflated/expanded configuration, inflatable member 31308 also may act as an anchor for medical device 31300 within a lung airway. In some embodiments, the fluid may be a cold fluid (e.g., a cryofluid) and may be circulated through inflatable member 31308. In some embodiments, the cryofluid may be circulated from a source outside of the patient toward inflatable member 31308 via an inflow lumen of member 31309, within inflatable member 31308, and out of inflatable member 31308 via an outflow lumen of member 31309, toward either the fluid source or to be discarded. This circulation may first reduce the temperature of the lung airways to damage one or more afferent or efferent nerves, while a subsequent therapy, e.g., acoustic energy, sonic energy, or HIFU, is applied by energy delivery element 31314 to damage the one or more afferent or efferent nerves. In some embodiments, the damaged afferent or efferent nerve may be less likely to regenerate than if an acoustic energy, sonic energy or HIFU treatment had been applied alone without cryogenic cooling.

In some embodiments, a barrier may be deployed on the inner or outer surface of the inflatable member 31308 to shield a portion of inflatable member 31308 from transmitting energy to tissue. For example, 270 degrees of the balloon could be shielded and 90 degrees could be designed to transmit HIFU energy to the tissue. This selective shielding of HIFU energy may be utilized in scenarios where the nerve location is known to reduce the effect of HIFU energy on surrounding tissues. In some embodiments, energy delivery element 31314 may be rotatable to selectively deliver HIFU to a partial (e.g., 90 degree) portion of the airway.

In some embodiments, fluid 31310 may only be utilized for inflation of inflatable member 31308, and may not be a cryofluid. In some embodiments, fluid 31310 may not circulate within inflatable member 31308.

Referring to FIG. 73, a medical device 31400 may include an bronchoscopic member 31402, channels 31406, expandable distal member 31408, and elongate member 31409 that are substantially similar to bronchoscopic member 31302, channels 31306, expandable distal member 31308, and elongate member 31309 described with reference to FIG. 72, except that expandable distal member 31408 may further include a plurality of pores 31410 for transmitting a gel or liquid (e.g., saline) 31412 to a lung airway wall. The gel or liquid may form an interface layer between an energy delivery element 31414 disposed within inflatable member 31408 and the lung airway wall, thereby improving the transmission of energy to the lung airway wall. In some embodiments, inflatable member 31408 may be a weeping balloon. Energy delivery element 31414 may be substantially similar to energy delivery element 31014 described with reference to FIG. 69.

Referring to FIG. 74, a medical device 31500 may include an bronchoscopic member 31502, channels 31506, expandable distal member 31508, and elongate member 31509 that are substantially similar to bronchoscopic member 31302, channels 31306, expandable distal member 31308, and elongate member 31309 described with reference to FIG. 72. Expandable or inflatable member 31508, such as, e.g., a balloon, may extend distally from distal end 31504 of bronchoscopic member 31502 via elongate member 31509. Inflatable member 31508 may be movable between an inflated/expanded configuration (shown in FIG. 74) and a deflated/collapsed configuration (not shown) in a substantially similar manner as inflatable member 1308. In some embodiments, inflatable member 31508 may include a plurality of pores (not shown) for transmitting a gel or liquid to a lung airway wall.

At least one energy delivery element 31514 may be disposed within inflatable member 31508. Energy delivery element 31514 is similar to energy delivery element 31014 described with reference to FIG. 69. In some embodiments, energy delivery element may rotate within elongate member 31508 via a rotating assembly 31516. Rotating assembly 31516 may be any suitable rotating assembly such as, e.g., a spool, wheel, crank, shaft, or the like. For example, element 31514 may be mounted on a bent wire that is anchored at one end at a distal center of member 31508. The wire extends through member 31509 and may be rotated at its proximal end outside the body, causing rotation of element 31514 about a central axis of member 31508. Thus, the rotation of energy delivery element 31514 via rotating assembly 31516 may allow energy delivery element 31514 to apply a partial or fully circumferential treatment to the lung airways. In some embodiments, a spiral treatment pattern can be applied to a lung airway by simultaneously rotating energy delivery element 31514, and moving elongate member 31509 longitudinally. In some embodiments, multiple energy delivery elements 31514 may be axially and/or circumferentially staggered within inflatable member 31508. The multiple energy delivery elements 31514 may be located on the same or on different rotating assemblies 31516. In some embodiments, after inflation of inflatable member 31508, energy delivery element 31514 may be rotated and utilized to scan for structures of interest (e.g., a nerve trunk or other targeted nerve). Once a nerve trunk or other targeted nerve is located, energy delivery element 31514 may be rotated to or secured to a circumferential location where treatment is desired. Energy delivery element 31514 then may transmit HIFU energy to the desired location.

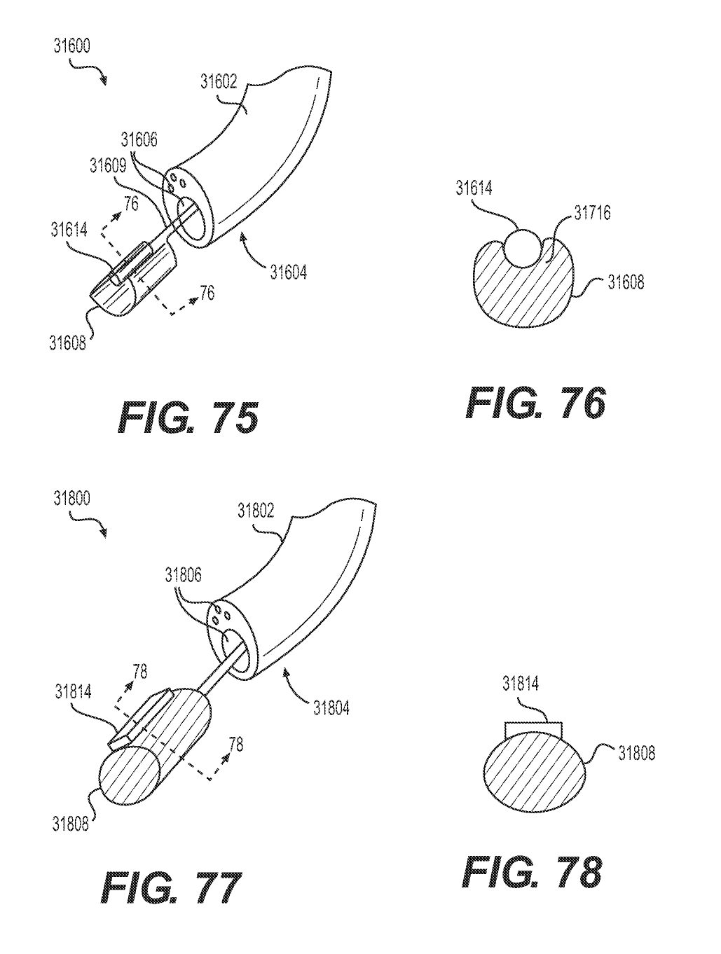

Referring to FIGS. 75 and 76, a medical device 31600 may include an bronchoscopic member 13602, channels 31606, and elongate member 31609 that are substantially similar to bronchoscopic member 31302, channels 31306, and elongate member 31309 described with reference to FIG. 72. An expandable or inflatable member 31608, such as, e.g., a balloon, may extend distally from distal end 31604 of bronchoscopic member 31602 via elongate member 31609. Inflatable member 31608 may be movable between an inflated/expanded configuration (shown in FIG. 75) and a deflated/collapsed configuration (not shown) in a substantially similar manner as inflatable member 31308. In some embodiments, inflatable member 31608 may include a plurality of pores (not shown) for transmitting a gel or liquid to a lung airway wall.

Inflatable member 31608 may include a groove 31716 (shown in FIG. 76) that is recessed into an outer surface of inflatable member 31608. Groove 31716 may be formed in any suitable shape, and may be configured to accept at least one energy delivery element 31614. Energy delivery element 31614 may be similar to energy delivery element 31014 described with reference to FIG. 69, but may disposed within groove 31716. In some embodiments, inflatable member 31608 may include a plurality of grooves 31716 for receiving multiple energy delivery elements 31614. In some embodiments, the additional grooves 31716 may be axially and/or circumferentially staggered from one another.

Referring to FIGS. 77 and 78, a medical device 31800 may include an bronchoscopic member 31802, channels 31806, an expandable or inflatable member 31808, and an elongate member 31809 that are substantially similar to bronchoscopic member 31302, channels 31306, inflatable member 31308, and elongate member 31309 described with reference to FIG. 72. Expandable or inflatable member 31808, such as, e.g., a balloon, may extend distally from distal end 31804 of bronchoscopic member 31802 via elongate member 31809. Inflatable member 31808 may be movable between an inflated/expanded configuration (shown in FIG. 77) and a deflated/collapsed configuration (not shown) in a substantially similar manner as inflatable member 31308. In some embodiments, inflatable member 31808 may include a plurality of pores (not shown) for transmitting a gel or liquid to a lung airway wall. At least one energy delivery element 31814 may be disposed on an outer surface of inflatable member 31808. Energy delivery element 31814 may be similar to energy delivery element 31014 described with reference to FIG. 69.

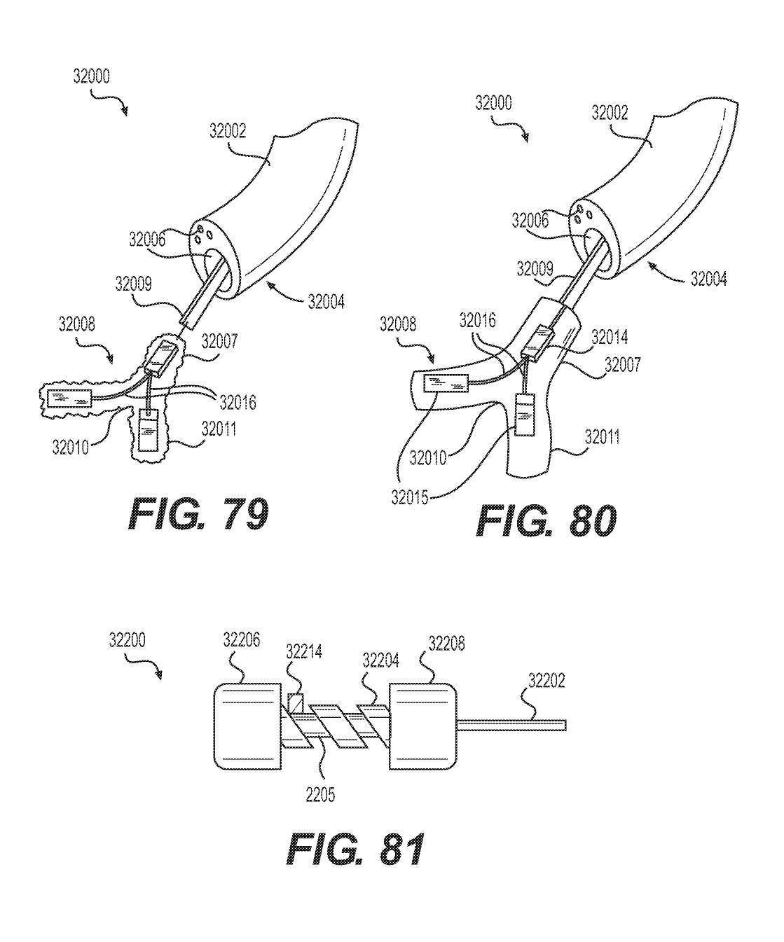

Referring to FIGS. 79 and 80, a medical device 2000 may include an bronchoscopic member 32002, channels 32006, and an elongate member 32009 that are substantially similar to bronchoscopic member 31302, channels 31306, and elongate member 31309 described with reference to FIG. 72. An expandable or inflatable member 32008, such as, e.g., a balloon, may extend distally from distal end 32004 of bronchoscopic member 2002 via elongate member 32009. Inflatable member 32008 may be movable between three positions: (1) a deflated/collapsed position wherein member 32008 is deflated and the device is compressed within lumen 32006 (i.e. linking members are adjacent one another) (not shown); (2) a deflated/expanded configuration (shown in FIG. 79) in which linking members 32016 are spread apart and member 32008 is deflated; and (3) an inflated/expanded configuration (shown in FIG. 80) in which linking members 32016 are spread apart and member 32008 is inflated. Inflatable member 32008 may extend distally from a proximal portion 32007, and subdivide into a plurality of branches 32011. A branch point 32010 may be disposed between a given pair of branches 32011. That is, inflatable member 32008 may be Y-shaped such that branch point 32010 can appose against a bodily branch point (such as branch point 510, 514 described with reference to FIG. 5). As shown in FIGS. 79 and 80, inflatable member 32008 is depicted with two branches 32011, but may include any additional number of branches. Medical device 32000 may be particularly useful and effective at treating branch points or bifurcations of lung airways.

A plurality of energy delivery elements may be disposed within inflatable member 32008. At least one first energy delivery element 32014 may be disposed within proximal portion 32007 of inflatable member 32008. A plurality of second energy delivery elements 32015 may extend distally from first energy delivery element 32014 via linking members 32016. Linking members 32016 may be self-expanding metal or metal alloy wires connected at one end to element 32014 and at the other end to an element 32015. When deployed from the lumen 32006, members 32016 may naturally spread apart. Each second energy delivery element 32015 may extend through a respective branch 32011 of inflatable member 32008, such that each second energy delivery element 32015 can apply energy to a different lung airway. In an alternative embodiment, linking members 32016 may be placed or pushed into position with inflatable member 32008 when inflatable member 32008 is inflated. Linking members 32016 may float freely within inflatable member 32008, or may alternatively may be coupled to inflatable member 32008 (e.g., at a distal end) to provide better control of energy delivery elements 32014 and 32015 within inflatable member 32008. In some embodiments, first energy delivery element 32014 may deliver energy to a first lung airway (e.g., a third generation lung airway), while second energy delivery elements 32015 each deliver energy to a respective lung airway distal to the first lung airway (e.g., to respective fourth generation lung airways). Energy delivery elements 32014 and 32015 may be axially translatable and rotatable in some embodiments.

In some embodiments, inflatable member 32008 may include a plurality of pores (not shown) for transmitting a gel or liquid to a lung airway wall. In some embodiments, one or more of energy delivery elements 32014 and 32015 may be disposed on an outer surface of inflatable member 32008. Energy delivery elements 32014 and 32015 may transmit energy in a substantially similar manner as energy delivery element 31314 described with reference to FIG. 72. In some embodiments, multiple energy delivery elements 32014 may be disposed within proximal portion 32007 of inflatable member 32008. In some embodiments, multiple energy delivery elements 2015 may be disposed within a given branch 32011. In some embodiments, one or more energy delivery elements 32014, 32015 may be disposed on an outer surface of inflatable member 32008.

A medical device 32200 is shown in FIG. 81. Medical device 32200 may include an elongate member 32202 that extends from an bronchoscopic member (not shown) into a lung airway. Medical device 32200 may include a distal inflatable member 32206 and a proximal inflatable member 32208, such as, e.g., balloons that are reciprocally movable between a collapsed configuration (not shown) and an expanded configuration shown in FIG. 81. Inflatable members 32206 and 32208 may serve to anchor medical device 32200 within a lung airway. A protrusion 32204 may be formed on an outer surface of elongate member 32202. Protrusion 32204 may be formed as a spiral, and together with elongate member 32202 may form a spiral track 32205 through which an energy delivery element 32214 may traverse through. In some embodiments, protrusion 32204 may be simultaneously or separately inflatable with inflatable members 32206 and 32208. In some embodiments, track 32205 may alternatively be formed as a recess within an outer surface of elongate member 32202. Thus, elongate member 32202 may deliver inflation fluid to one or more of protrusion 32204, and inflatable members 32206 and 32208. In some embodiments, elongate member 32202 may include an actuator, e.g., a wire that is coupled to energy delivery element 32214. In some embodiments, as the actuator wire is pulled proximally, energy delivery element 32214 may move proximally through track 32205 to provide a spiral treatment to a lung airway wall. Similarly, the actuator may be moved distally to move energy delivery element 32214 distally through track 32205. While depicted as spirals, protrusion 32204 and track 32205 may be formed in any other suitable configuration. Alternatively, the actuator may simply position energy delivery element at a desired axial and radial position prior to energy delivery. Track 32205 may include undercuts or other suitable mating features, into which protrusions or mating features of energy delivery element 32214 mate. The respective mating features may facilitate the movement of energy delivery element 32214 along track 32205, and may prevent energy delivery element 32214 from disengaging track 32205. In another aspect, a fluid, e.g., saline, may be injected into the space between two inflatable members during therapy to improve energy transmission efficiency to the airway wall.

Referring back to FIGS. 67 and 68, medical devices 6600 and 6700 may be configured to deliver HIFU in addition or alternative to a cryotherapy. For example, active regions 6606 and 6706 may be energy delivery elements substantially similar to energy delivery elements 31014 described with reference to FIG. 69.

Radio Frequency (RF)

In some embodiments, RF energy may be applied to tissues defining or otherwise surrounding a lung airway in a controlled manner to damage afferent sensory nerves, efferent nerves, or the like. The RF energy may be configured to damage or destroy nerve function, including the ability of a targeted nerve to transmit signals.

Delivery of the RF energy may be through the right main bronchus, left main bronchus, or both, as treating only one of the right or left main bronchi may be sufficient for a significant reduction in bronchoconstriction and/or mucus production, as the right and left vagus nerves traverse along the right and left main bronchi, respectively.

Linking the delivery of RF energy with a detection system such as, e.g., an electrode mapping catheter, to a localized treatment location may allow for a more specific treatment to be applied, potentially reducing the damage to adjacent tissues. Other imaging procedures, such as, e.g., magnetic resonance imaging (MRI), diagnostic sonography, or other suitable imaging techniques also may be used.

The medical devices of the present disclosure, e.g., medical devices 8200, 8300, 8500, 8700, 8800, 8900, 9100, 9300, 1700, 1900, 2900, 3000, 3100, 6700, 6800, 31000, 31100, 31200, 31600, and 31800, may deliver RF energy to locally treat (e.g., ablate) lung airway tissue or lung airway nerves. Delivery of RF energy may be via a minimally-invasive procedure that can treat tissue located at a depth below the lung airway surface.

In some embodiments, a location of the nerve(s) to be targeted in the airways may be determined by direct visualization, of, e.g., an anatomical structure, by ultrasound scanning/imaging, or by any other suitable means. Once a targeted nerve or treatment location is determined, the medical device may deliver RF energy to the targeted nerve or treatment location. In one embodiment, the targeted nerve or treatment location may be first detected by ultrasound scanning/imaging, and then RF energy may be delivered to a less than 360 degree circumference, e.g., a less than 90 degree circumference of the airway which corresponds with the target nerves or treatment location. In other embodiments, RF energy may be applied to an entire 360 degree circumference of the airway (e.g., in spiral treatment patterns). In some embodiments, little or no damage will be caused to the remaining circumference of the airways that are not targeted by the medical device. In some embodiments, the medical device may be capable of both imaging and delivering a therapy. Alternatively, the medical device may be configured for energy delivery around a larger circumference of the airway or esophagus, and may be directed at additional locations other than nerve tissue. In some embodiments, the medical device may direct RF energy toward smooth muscle tissue in the lung airways to achieve reduced bronchoconstriction (by e.g., scarring the smooth muscle tissue). In some embodiments, the medical device may direct RF energy toward tissues and body elements affecting other diseases such as, e.g., asthma, chronic cough, chronic bronchitis, and Cystic Fibrosis, where bronchoconstriction, mucus hypersecretion, and cough are also observed.

The medical devices also may be formed of a radiopaque material so that they can be visualized under fluoroscopic guidance, or otherwise include radiopaque or other imaging markers for guidance. The markers may be used to ensure that a correct direction of therapy is applied. In some embodiments, the medical device may be prevented from activating until the marker is appropriately positioned.

In some embodiments, medical devices may include one or more sensors to detect various parameters or anatomical structures. In one embodiment, the one or more sensors may include temperature sensors configured to detect a presence/amount of therapy delivered. In another embodiment, the one or more sensors may include structures within the lung airway configured to use Doppler ultrasound to detect blood vessels. In another embodiment, the one or more sensors may sense electrical measurement of nerve traffic that corresponds to an efficacy of the treatment. In another embodiment, the one or more sensors may include a vision system for direct observation. In yet another embodiment, the one or more sensors may include a force transducer, strain gauge, or similar sensor to measure radial force in the lung airways. One or more feedback mechanisms (e.g., PID, fuzzy logic, or the like) may be utilized to control the intensity of RF energy applied, and thus the extent of damage to lung tissues and lung nerves. In some embodiments, IR measurement, tissue optical parameter measurement (e.g., reflectance, color, scattering), direct temperature measurement (e.g., using thermocouples), or other suitable mechanisms may be utilized to measure the temperature change of lung tissue in response to the applied RF energy.

Controller 32 may be coupled to or otherwise include an RF energy source. In various examples of the present disclosure, RF energy may be applied to tissues defining a body lumen (e.g., a lung airway) for a length of time in the range of about 0.1 seconds to about 600 seconds. In one example, a power source may be capable of delivering about 1 to 100 watts of RF energy, and may possess continuous flow capability. The tissues defining a lung airway may be maintained at a temperature that is lesser than, equal to, or greater than ambient body temperature. In one example, the tissues may be maintained at at least about 60° C., between 70° C. to 95° C., and/or between 70° C. to 85° C. The RF power-level may generally range from about 0-30 W, or another suitable range. In some examples, the power source may operate at up to a 75° C. setting. In some examples, RF energy may be delivered in discrete activations of, e.g., 5 to 10 seconds per activation. The frequency of the RF energy may be from 300 to 1750 kHz. It should be noted that, in at least some examples, other suitable values for energy delivery times, wattage, airway temperature, RF electrode temperature, and RF frequency are also contemplated.

In some examples, to avoid excessive epithelial damage during therapy, while still achieving the depth of penetration necessary to target the nerves located approximately 1 to 2 mm beneath the epithelial surface, a cooled electrode may be used in conjunction with any of medical devices 8200, 8300, 8500, 8700, 8800, 8900, 9100, 9300, 1700, 1900, 2900, 3000, 3100, 6700, 6800, 31000, 31100, 31200, 31600, and 31800.

For example, a medical device 8200 is shown in FIG. 82. Medical device 8200 may include a first elongate member 8202 that extends from a proximal end (not shown) toward a distal end 8204. First elongate member 8202 may be a sheath, catheter, or other suitable elongate member configured to be inserted through a bronchoscope. In some examples, first elongate member 8202 may be a bronchoscope or other endoscopic member. A second elongate member 8206 may extend distally from distal end 8204 of first elongate member 8202. Second elongate member 8206 may include at least one energy delivery element 8208 configured to deliver RF energy to tissues of the lung. It is also contemplated that in some examples, any other suitable elongate member and/or energy delivery element may extend distally from first elongate member 8202. First elongate member 8202 may be configured to deliver a coolant to lumens of the body (e.g., lung airways) to absorb heat from the body lumens and/or from tissues defining or otherwise surrounding the body lumens (e.g., airway walls and the like). In some examples, coolant may be delivered by one or more lumens. Suitable examples of coolant include, but are not limited to, gases (e.g., air), liquids (e.g., saline), or other suitable sources of coolant.

Coolant may be directed generally into the body lumens or toward specific locations within the body lumens. In one example, coolant may be directed along the active electrode portions of a medical device. In other examples, coolant may be directed toward the electrode/tissue interfaces. In some examples, coolant may be circulated through the electrode without exiting the catheter or directly contacting tissues of the airway. In some examples, coolant may be delivered to the body lumens in a delivery step, and may be removed from the body lumen in a suction or withdrawal step.

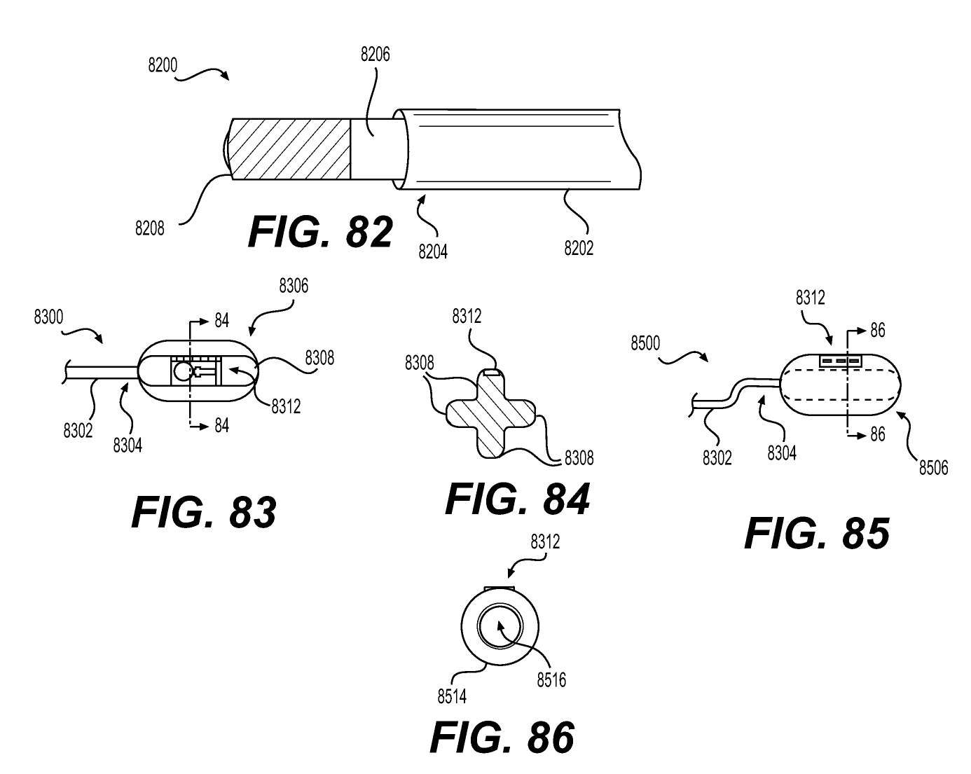

A medical device 8300 is shown in FIGS. 83 and 84. Medical device 8300 may include an elongate member 8302 that extends from a proximal end (not shown) toward a distal end 8304. An expandable member 8306 may be disposed at distal end 8304 of elongate member 8302. In some examples, expandable member 8306 may be reciprocally movable between a collapsed configuration (not shown) and an expanded configuration (shown in FIG. 83). Expandable member 8306 may include a plurality of radially outermost portions 8308 that are radially spaced from one another. Expandable member 8306 may include any suitable number of radially outermost portions 8308. In the example shown in FIGS. 83 and 84, expandable member 8306 includes four radially outermost portions 8308 that are spaced apart from one another by about 90 degrees, generally forming a cross-shaped or X-shaped cross-section when expandable member 8306 is in the expanded configuration. In the expanded configuration of medical device 8300, the radially outermost portions 8308 may be spaced apart from one another such that medical device 8300 does not occlude a body lumen or airway that it is disposed through. That is, the outer surface of expandable member 8306 may contact only a partial circumference of a body lumen or airway that it is disposed through. Medical device 8300 may have one or more energy delivery elements 8312 that are configured to deliver RF energy to tissues of the body. In some examples, each radially outermost portion 8308 may include an energy delivery element 8312. In other examples, each radially outermost portion 8308 may include a plurality of energy delivery elements 8312. It is further contemplated that energy delivery elements 8312 may be positioned about the various radially outermost portions 8308 of medical device 8300 to deliver various patterns of therapy such as, e.g., spiral, longitudinal, staggered, or other suitable treatment patterns. It is further contemplated that one or more radially outermost portions 8308 may not include an energy delivery element 8312, or in some modes of energy delivery, that one or more energy delivery elements 8312 may not be activated at a given time. Energy delivery elements 8312 may be coupled to radially outermost portions 8308 by any suitable mechanism. In some examples, energy delivery elements 8312 may be printed onto the outer surface of radially outermost portions 8308.

Elongate member 8302 may include one or more lumens configured to convey a fluid to and from expandable member 8306. For example, elongate member 8302 may include a first lumen (not shown) configured to convey fluid from a fluid source toward the expandable member 8306 to move expandable member 8306 from the collapsed configuration to the expanded configuration. Elongate member 8302 also may include a second lumen (not shown) that is configured to convey fluid from expandable member 8306 back toward the fluid source or to another suitable receptacle. In some examples, the first and second lumens may be operated simultaneously to move expandable member 8306 from the collapsed configuration to the expanded configuration, and also to maintain expandable member 8306 in the expanded configuration. In another example, the same lumen may convey fluid to and from expandable member 8306.

In some examples, elongate member 8302 also may include a third lumen, separate from the first and second lumens, the third lumen being configured to deliver power to energy delivery elements 8312 via, e.g., an energizing member or wire. Additional features of the device (e.g., temperature sensing elements, other sensor elements such as pressure or fluid sensors) may utilize different lumens for different sensor leads, and/or may utilize separate or the same lumen(s) for fluid conveyance or for blowing gas (e.g., pressurized air, hot air) into the airway to move or desiccate secretions (e.g., mucus). In addition, the lumen(s) may be used to simultaneously or sequentially deliver fluids and/or suction fluid to assist in managing the moisture within the passageway. Such management may optimize the electrical coupling of an energy delivery element to the tissue (by, for example, altering impedance). In other examples, the lumens may be utilized to provide a cooling fluid to expandable member 8306 and/or the airway.

A medical device 8500 is shown in FIGS. 85 and 86. Medical device 8500 may be substantially similar to medical device 8300 except that medical device 8500 may include an expandable member 8506 instead of expandable member 8306. Expandable member 8506 may be disposed at distal end 8304 of elongate member 8302. In some examples, expandable member 8506 may be reciprocally movable between a collapsed configuration (not shown) and an expanded configuration (shown in FIG. 85). Expandable member 8506 may include an outer surface 8514 that may contact tissues defining a body lumen (e.g., airway) when expandable member 8506 is in the expanded configuration. In some examples, the outer surface 8514 of expandable member 8506 may contact the entire circumference (or substantially all of the circumference) of a body lumen or airway that it is disposed through. Medical device 8500 may have one or more energy delivery elements 8312 that are configured to deliver RF energy to tissues of the body. Expandable member 8506 may include a lumen 8516 disposed through a middle portion of expandable member 8506. While in the expanded configuration, medical device 8500 may not occlude a body lumen or airway that it is disposed through, due to the presence of lumen 8516. One or more energy delivery elements 8312 may be disposed on outer surface 8514 of expandable member 8506. The one or more energy delivery elements 8312 may be disposed on outer surface 8514 in any suitable pattern, including in the same patterns described with reference to FIGS. 83 and 84. Lumen 8516 also may be utilized to deliver a medical instrument, fluid, drug, or other entity distally of expandable member 8506.

While it may be clinically acceptable to occlude a body lumen or airway during energy delivery, it may be advantageous to avoid occlusion to reduce the risk of adverse events occurring, particularly in patients of poor health. Thus, the use of medical devices 8300 and 8500 may be beneficial in that they may permit RF energy delivery in combination with cooling, while still avoiding the occlusion of a body lumen or airway. Other mechanisms for preventing the occlusion of the body lumen or airway during energy delivery include, stents, baskets, spiral shaped catheters, and the like. It is further contemplated that the expandable members 8306 and 8506 may be utilized as the expandable or inflatable member in other examples described by the present disclosure to deliver any suitable energy modality, such as, e.g., neurolytic agents, HIFU, cryo, laser, RF, microwave, or the like. Expandable members 8306 and 8506 may permit the passage of liquids, gases, and other materials through a body lumen or airway, and distally of the expandable members 8306, 8506, while in the expanded configuration, and during energy delivery.

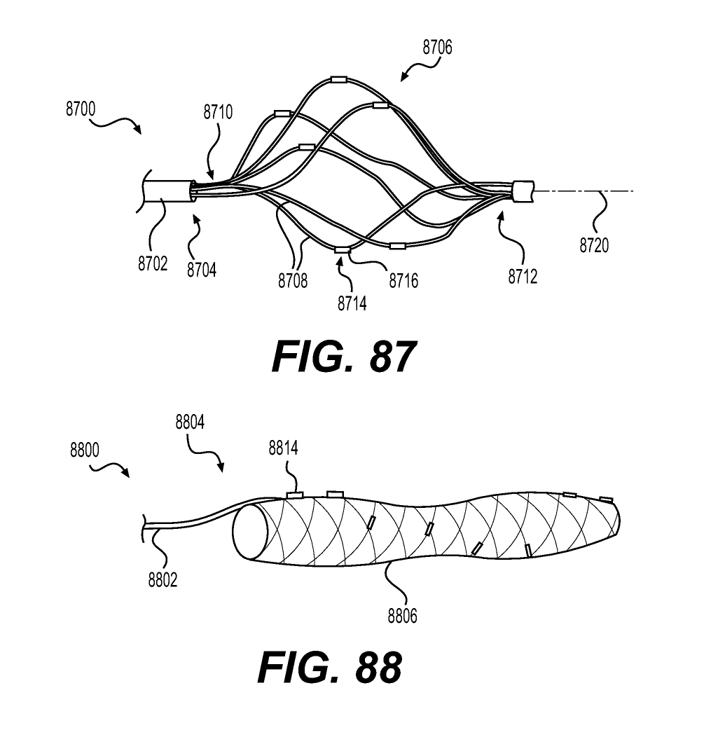

A medical device 8700 is shown in FIG. 87. Medical device 8700 may include an elongate member 8702 that extends from a proximal end (not shown) toward a distal end 8704. An expandable member 8706 may be disposed at distal end 8704 of elongate member 8702. Expandable member 8706 may be movable between a collapsed configuration (not shown) and an expanded configuration shown in FIG. 87. Expandable member 8706 may include one or more expandable legs 8708 that extend longitudinally from a proximal end 8710 of expandable member 8706 toward a distal end 8712 of expandable member 8706. In the example shown in FIG. 87, expandable member 8706 may include six expandable legs 8708, although any other suitable number also may be utilized. In some examples, each expandable leg 8708 may spiral, coil, or form a helix as it extends from proximal end 8710 toward distal end 8712. Legs 8708 may generally converge toward one another at both proximal end 8710 and distal end 8712. In some examples, the curvature of one or more expandable legs 8708 may bow radially outward from a longitudinal axis 8720 of medical device 8700 toward a radially outermost portion 8714. Radially outermost portion 8714 may include one or more energy delivery elements 8716. Energy delivery elements 8716 may be substantially similar to energy delivery elements 8312 described above. In some examples, adjacent expandable legs 8708 may possess different curvatures such that adjacent radially outermost portions 8714 are longitudinally and/or radially staggered from one another. In the example of FIG. 87, the six radially outermost portions 8714 are positioned relative to one another such that medical device 8700 may deliver energy to tissue in a substantially spiral manner. It is further contemplated that each expandable leg 8708 may be hollow and internally-cooled via circulation of a cooling fluid.

A medical device 8800 may include an elongate member 8802 that extends from a proximal end (not shown) toward a distal end 8804. An expandable member 8806 may be disposed at the distal end 8804 of the elongate member 8802. In the example shown, the expandable member 8806 may include a stent (e.g., a braided stent) that is configured to move between a collapsed configuration and an expanded configuration. One or more energy delivery elements 8814 may be disposed on the outer surface of the expandable member 8806 in any suitable configuration or pattern. Energy delivery elements 8814 may be substantially similar to energy delivery elements 8312 described herein. In the example shown, a plurality of energy delivery elements 8814 are disposed on the outer surface of the expandable member 8806 in a spiral configuration. In other examples, energy delivery elements 8814 may be incorporated into expandable member 8806 itself, and may be defined by insulated and/or nonconductive regions. In some examples, the entirety of expandable member 8806 may be formed as an electrode and insulated coatings may be placed on the nonconductive regions. Alternatively, the entirety of expandable member 8806 may be coated with insulation, which can be selectively removed to expose one or more conductive regions. The expandable member 8806 may be configured to deliver energy therapy to airways of varying diameters and dimensions, by expanding until constrained by the walls of a given body lumen or airway.

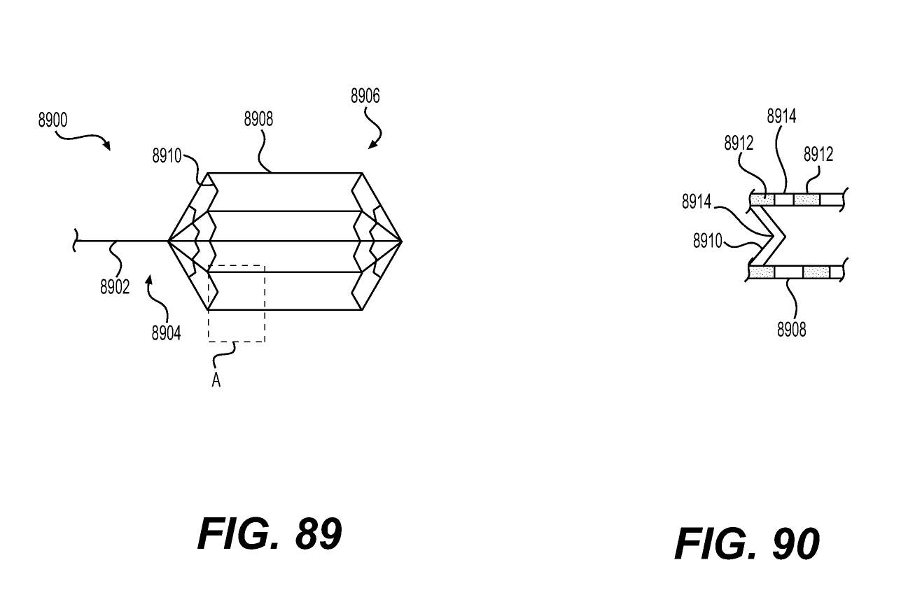

A medical device 8900 is shown in FIGS. 89 and 90. Medical device 8900 may include an elongate member 8902 that extends from a proximal end (not shown) toward a distal end 8904. An expandable member 8906 may be disposed at the distal end 8904 of the elongate member 8902. In the example shown, the expandable member 8906 may include a stent or basket that is configured to move between a collapsed configuration and an expanded configuration. Expandable member 8906 may include one or more longitudinally extending legs 8908. Legs 8908 may be parallel to one another and to a longitudinal axis at the center of expandable member 8906. The proximal ends of legs 8908 may converge toward one another, while the distal ends of legs 8908 also converge toward one another. In some examples, adjacent legs 8908 may be coupled to one another by one or more bent arms 8910. In some examples, the bent arms 8910 may only be disposed at tapered proximal and/or distal portions of legs 8908. Bent arms 8910 may provide for improved control and relative uniformity of spacing between adjacent legs 8908, thereby improving the control of treated tissue. Expandable member 8906 may include one or more energy delivery elements 8912 that are substantially similar to energy delivery elements 8312 described above. In one example, each leg 8908 may include a plurality of energy delivery elements 8912 that are spaced apart from one another along a longitudinal axis of a given leg 8908. It is also contemplated that arms 8910 may include one or more energy delivery elements 8912. In some examples, temperature sensing elements 8914 may be disposed on legs 8908 between energy delivery elements 8912, or on arms 8910. During energy delivery therapy, RF energy may flow from any given energy delivery element 8912 to any other energy delivery element 8912. For example, RF energy may flow from one energy delivery element 8912 toward another energy delivery element 8912 disposed on the same or different leg 8908.

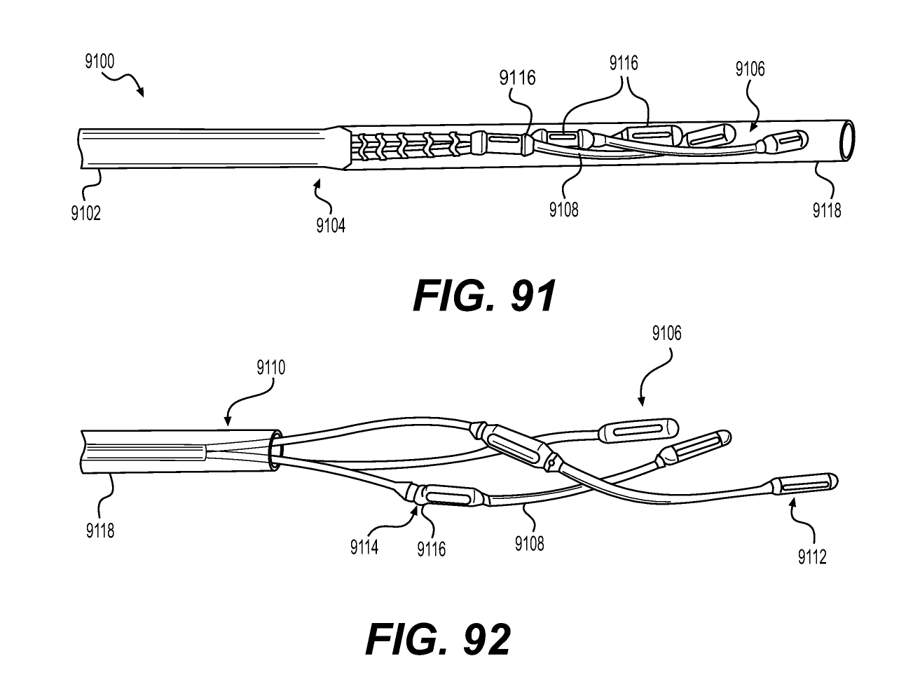

A medical device 9100 is shown in FIGS. 91 and 92. Medical device 9100 may include an elongate member 9102 that extends from a proximal end (not shown) toward a distal end 9104. An expandable member 9106 may be disposed at distal end 9104 of elongate member 9102. Expandable member 9106 may be movable between a collapsed configuration (shown in FIG. 91) and an expanded configuration shown in FIG. 92. Expandable member 9106 may include one or more expandable legs 9108 that extend longitudinally from a proximal end 9110 toward a distal end 9112. A sheath 9118 may be disposed around expandable member 9106, and may constrain expandable member 9106 in the collapsed configuration. In some examples, sheath 9118 may be pulled proximally relative to expandable member 9106 to allow expandable member 9106 to move from the collapsed configuration to the expanded configuration. In the example shown in FIGS. 91 and 92, expandable member 9106 may include three expandable legs 9108, although any other suitable number also may be utilized. Expandable legs 9108 may converge toward one another at proximal end 9110, but may be unconnected at distal end 9112 and all points between proximal and distal ends 9110 and 9112. In some examples, each expandable leg 9108 may spiral, coil, or form a helix as it extends from proximal end 9110 toward distal end 9112. In some examples, the curvature of one or more expandable legs 9108 may bow radially outward from a longitudinal axis of medical device 9100 toward a radially outermost portion 9114. Radially outermost portion 9114 may include one or more energy delivery elements 9116. Energy delivery elements 9116 may be substantially similar to energy delivery elements 8312 described above. In some examples, adjacent expandable legs 9108 may possess different curvatures such that adjacent radially outermost portions 9114 are longitudinally and/or radially staggered from one another. It is further contemplated that one or more of expandable legs 9108 may be hollow and internally-cooled via a cooling fluid. For example, one or more of expandable legs 9108 may include at least two lumens for fluid circulation.

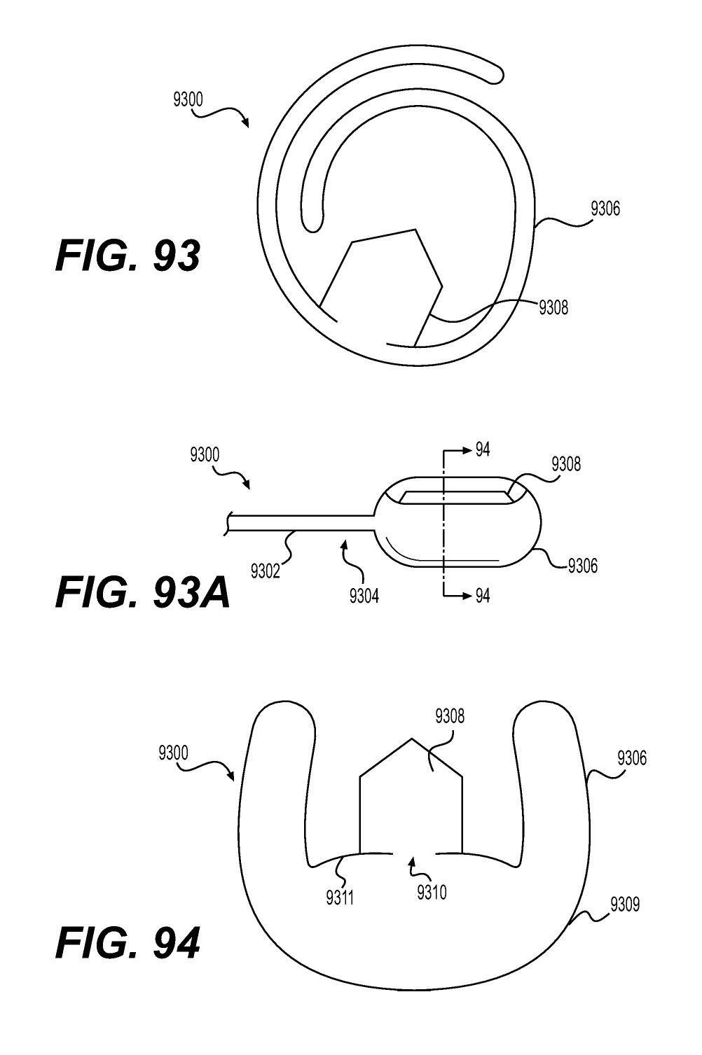

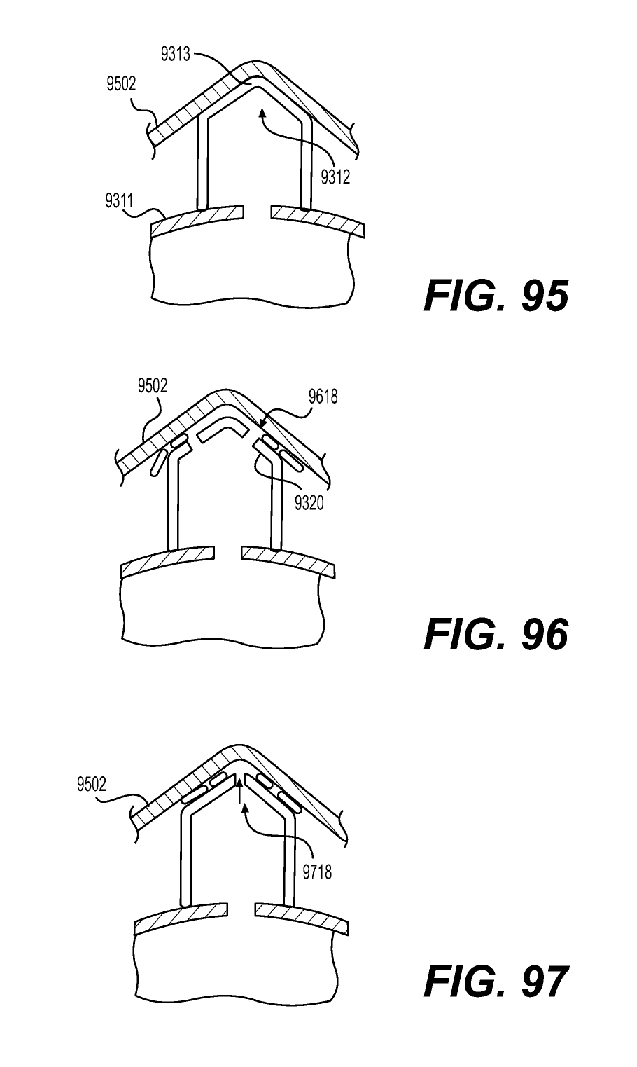

A medical device 9300 is shown in FIGS. 93-95. Medical device 9300 may include an elongate member 9302 that extends from a proximal end (not shown) toward a distal end 9304. An expandable member 9306 may be disposed at distal end 9304 of elongate member 9302. In some examples, expandable member 9306 may be reciprocally movable between a collapsed configuration (shown in FIG. 93, a partially-expanded configuration (shown in FIGS. 93A and 94), and an expanded configuration (shown in FIG. 95). In the collapsed configuration, expandable member 9306 may be configured to move within, e.g., a bronchoscope. Expandable member 9306 may have a smaller diameter when disposed on the collapsed configuration that when disposed in either the partially-expanded or expanded configurations. In the partially-expanded configuration, energy delivery element 9308 may be at least partially recessed with respect to a remainder of expandable member 9306. For example, expandable member 9306 may include an outer surface 9309, and energy delivery element 9308 may extend from a portion 9311 of outer surface 9309. The portion 9311 may be disposed closer to a radial center of expandable member 9306 in the partially-expanded configuration compared to a remainder of outer surface 9309. In other words, the portion 9311 may be recessed within expandable member 9306 in the partially-expanded configuration. Medical device 9300 may be positioned in the partially-expanded configuration. For example, once in the partially-expanded configuration, a user may rotate expandable member 9306 to properly position energy delivery element 9308. Once the proper position is attained, expandable member 9306 may be moved to the fully-expanded configuration.

When expandable member 9306 is fully expanded, energy delivery element 9308 and portion 9311 of outer surface 9309 may extend radially outward such that the entirety of outer surface 9309 forms a substantially continuous circumference, except for the portion of outer surface 9309 that energy delivery element 9308 extends from. Expandable member 9306 may expand and contract via the delivery of an inflation and/or cooling fluid from a proximal end of medical device 9300. Medical device 9300 may include an energy delivery element 9308. Energy delivery element 9308 may be configured to delivery RF energy to tissues of the body in a substantially similar manner as the other energy delivery elements described herein. In some examples, energy delivery element 9308 may be hollow, and in fluid communication with expandable member 9306 via a conduit 9310. Thus, inflation and/or cooling fluid may circulate through expandable member 9306 while expandable member 9306 is in the expanded configuration, and during energy delivery therapy.

Energy delivery element 9308 may be formed as a blade with a bladed and inclined tip 9312. Tip 9312 may be tapered to meet at a ridge or blade 9313. In some examples, energy delivery element 9308 may include serrations or other piercing features, if desired. The bladed configuration of energy delivery element 9308 may allow energy delivery element 9308 to apply pressure to tissue when expandable member 9306 is in the expanded configuration. In some examples, this may allow energy delivery element 9308 to be disposed closer to tissues that are located radially outward of a body lumen through which energy delivery element 9308 is disposed. In one example, the bladed configuration of energy delivery element 9308 may allow energy delivery element 9308 to be disposed closer to a pulmonary nerve by applying a blunted force to and pushing tissue wall 9502. Bladed tip 9312 may be blunted or otherwise atraumatic to prevent excessive damage to tissues defining a body lumen (e.g., lung epithelium). While medical device 9300 is shown with a single energy delivery element 9308 in FIGS. 93-95, it is contemplated that any suitable number of energy delivery elements 9308 may be disposed on the outer surface of expandable member 9306. For example, one or more energy delivery elements 9308 may be longitudinally and/or radially spaced from one another. In another example, energy delivery elements 9308 may be staggered from one another so as to deliver a spiral, circumferential, radial, or other suitable treatment. FIG. 96 depicts energy delivery element 9308 with one or more outlets 9618 disposed on inclined edges 9620 of bladed tip 9312. FIG. 97 depicts energy delivery element 9308 having an outlet 9718 disposed on the apex of bladed tip 9312, where inclined edges 9320 converge. Outlets 9618 and 9718 may be configured to deliver cooling fluid from expandable member 9306 to surface tissues defining a body lumen (e.g., lung epithelial tissue) to help prevent or reduce damage to those tissues during energy delivery therapies, particularly those therapies targeting tissues disposed radially outward of the surface tissues, away from the airway.

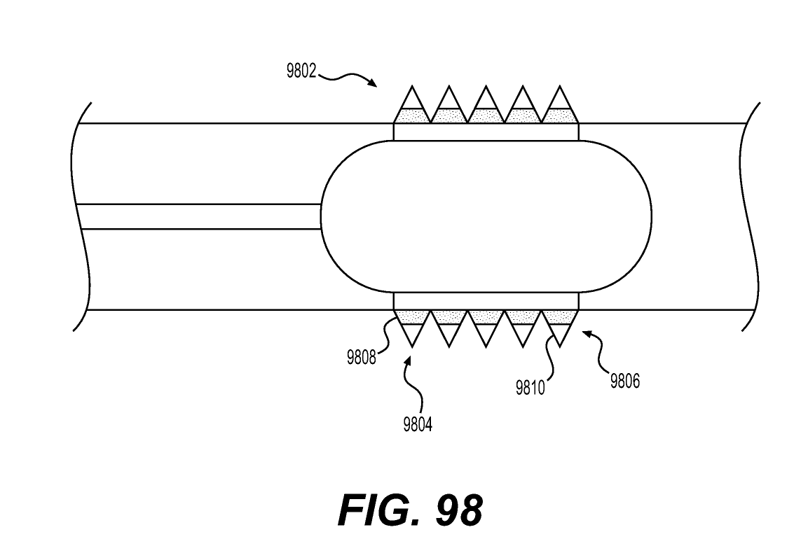

FIG. 98 depicts an energy delivery element 9802 and an energy delivery element 9804 in accordance with an example of the present disclosure. Energy delivery elements 9802 and 9804 may be substantially similar to one another, or may be different than one another in one or more aspects. Energy delivery element 9802 may include one or more protrusions 9806 that are configured to pierce through tissue. Protrusions 9806 may be formed as a spike, blade, or other suitable piercing member, and may be longitudinally and/or radially arranged on each energy delivery element 9802. Protrusions 9806 may include a proximal portion 9808 and a distal portion 9810. Proximal portion 9808 may be electrically insulated, while distal portion 9810 may be electrically active. In some examples, both proximal portion 9808 and distal portion 9810 may be configured to pierce through tissue. Such a configuration may allow energy delivery element 9802 to treat tissues that are disposed radially outward of the surface tissue 9812 (such as, e.g., nerve tissue), while reducing the amount of therapy applied to or damage incurred by the surface tissue 9812. Energy delivery element 9802 may be used in a monopolar energy delivery configuration, or may be utilized in a bipolar manner. In some examples, RF current may flow from energy delivery element 9802 toward energy delivery element 9804, or vice versa. It is contemplated that the configuration of energy delivery elements 9802 and 9804 may apply to any energy delivery element of the present disclosure.

It is also contemplated that one or more RF energy delivery elements can be incorporated into various medical devices described herein. For example, referring back to FIGS. 10-12, medical device 1000 may include one or more RF energy delivery elements that are disposed on or adjacent to fluid delivery device 1010.

Referring back to FIGS. 17-19, medical devices 1700 and 1900 may include one or more RF energy delivery elements incorporated with fluid delivery devices 1712. In some examples, the RF energy delivery elements may be incorporated on fluid delivery devices 1712 at a location proximal to distal stop 1714. However, it is also contemplated that RF energy delivery elements may be incorporated on fluid delivery devices 1712 at a location distal to distal stop 1714 in order to be positioned closer to, e.g., nerve tissues or other tissues disposed radially outward of airway wall 102 or a surface tissue. Referring to FIGS. 29-37, it is also contemplated that an RF energy delivery element may be incorporated into one or more of fluid delivery devices 2912 and/or 3410 in a similar manner as described with reference to fluid delivery device 1010.

Fluid delivery devices 1010, 1712, 2912, and/or 3410 may be configured to pierce through airway tissue to deliver an RF energy delivery element closer to, e.g., nerve tissue, while still maintaining the ability to deliver neurolytic agents and other substances through airway tissue. In one example, in addition to or instead of delivering neurolytic agents, the fluid delivery devices may be configured to deliver one or more cooling fluids (such as, e.g., saline or water), therapeutic agents, or another suitable agent.

Referring to FIGS. 67 and 68, it is contemplated that in addition to or alternative to the delivery of cryo energy to the airway walls (e.g., transfer of energy from the airway walls), that active regions 6606 and/or 6706 each may include an RF energy delivery element configured to deliver RF energy to tissue. In such embodiments, cooling fluids may be circulated through one or more of elongate member 6602 and 6702, and/or expandable member 6610.

Referring to FIGS. 69-71, it is contemplated that energy delivery elements 31014 and 31214 may additionally or alternatively be configured to deliver RF energy to tissue. In other examples, active portions of legs 31008, mesh 31116, and/or expandable member 31208 may be configured to deliver RF energy to tissue. In such examples, the active portions of legs 31008, mesh 31116, and/or expandable member 31208 may be defined by insulated and/or nonconductive regions that are not configured to deliver RF energy to tissue.

Referring to FIGS. 75-78, it is contemplated that energy delivery elements 31614 and 31814 may additionally or alternatively be configured to deliver RF energy to tissue. In such embodiments, cooling fluids may be circulated through one or more of inflatable members 31608 and 31808.

In some examples, any of the aforementioned medical devices configured to deliver RF energy, may alternatively be configured to deliver irreversible or reversible electroporation therapies, which are electrical methods of causing cell death by apoptosis. In some examples, there may be 10 to 100 pulses per treatment, with a pulse length of 1 millisecond to 1 microsecond. There may be 100 to 1000 milliseconds between pulses, with a field strength from 250 to 3000 volt/cm. Pulses may be configured as high frequency bursts of 250 to 500 kHz to avoid muscle fiber contractions. In some examples, electroporation therapy may be more effective in treating non-myelinated nerve fibers (e.g., afferent sensor fibers), although other suitable target tissues are also contemplated.

It is further contemplated that any of the medical devices disclosed herein may additionally include one or more temperature sensing elements configured to sense a temperature of tissue and/or of the energy delivery element. The temperature sensing elements may be configured to directly contact tissue in some examples, and may be configured to apply ablative energy. In such examples, the temperature sensing element may rapidly switch between an ablation mode and a temperature sensing mode. It is further contemplated that temperature sensing elements may be configured to deliver ablation energy and sense temperature simultaneously. Any of the medical devices described herein may additionally include orifices or pores in the energy delivery element surfaces to allow some amount of coolant fluid to exit and contact the tissue in the immediate vicinity of the energy delivery element. The coolant fluid, in clinically acceptable amounts, may not need to be suctioned out or removed from the airway. Or, the coolant may be subsequently suctioned or otherwise removed from the airway.

Microwave Energy

In some embodiments, microwave energy may be applied to tissues defining or otherwise surrounding a lung airway in a controlled manner to damage afferent sensory nerves, efferent nerves, or the like. The microwave energy may be configured to damage or destroy nerve function, including the ability of a targeted nerve to transmit signals.

Delivery of the microwave energy may be through the right main bronchus, left main bronchus, or both, as treating only one of the right or left main bronchi may be sufficient for a significant reduction in bronchoconstriction and/or mucus production, as the right and left vagus nerves traverse along the right and left main bronchi, respectively.

Linking the delivery of microwave energy with a detection system such as, e.g., an electrode mapping catheter, to a localized treatment location may allow for a more specific treatment to be applied, potentially reducing the damage to adjacent tissues. Other imaging procedures, such as, e.g., magnetic resonance imaging (MRI), diagnostic sonography, or other suitable imaging techniques also may be used.

Medical device 9900 may deliver microwave energy to locally ablate lung airway tissue or lung airway nerves. Delivery of microwave energy may be via a minimally-invasive procedure that can ablate tissue located at a depth below the lung airway surface.

In some embodiments, a location of the nerve(s) to be targeted in the airways may be determined by direct visualization, of, e.g., an anatomical structure, by ultrasound scanning/imaging, or by any other suitable means. Once a targeted nerve or treatment location is determined, the medical device may deliver microwave energy to the targeted nerve or treatment location. In one embodiment, the targeted nerve or treatment location may be first detected by ultrasound scanning/imaging, and then microwave energy may be delivered to a less than 360 degree circumference, e.g., a less than 90 degree circumference of the airway which corresponds with the target nerves or treatment location. In other embodiments, microwave energy may be applied to an entire 360 degree circumference of the airway (e.g., in spiral treatment patterns). In some embodiments, little or no damage will be caused to the remaining circumference of the airways that are not targeted by the medical device. In some embodiments, the medical device may be capable of both imaging and delivering a therapy. Alternatively, the medical device may be configured for energy delivery around a larger circumference of the airway or esophagus, and may be directed at additional locations other than nerve tissue. In some embodiments, the medical device may direct microwave energy toward smooth muscle tissue in the lung airways to achieve reduced bronchoconstriction (by e.g., scarring the smooth muscle tissue). In some embodiments, the medical device may direct microwave energy toward tissues and body elements affecting other diseases such as, e.g., asthma, chronic cough, chronic bronchitis, and Cystic Fibrosis, where bronchoconstriction, mucus hypersecretion, and cough are also observed.

The medical devices also may be formed of a radiopaque material so that they can be visualized under fluoroscopic guidance, or otherwise include radiopaque or other imaging markers for guidance. The markers may be used to ensure that a correct direction of therapy is applied. In some embodiments, the medical device may be prevented from activating until the marker is appropriately positioned.

In some embodiments, medical devices may include one or more sensors to detect various parameters or anatomical structures. In one embodiment, the one or more sensors may include temperature sensors configured to detect a presence/amount of therapy delivered. In another embodiment, the one or more sensors may include structures within the lung airway configured to use Doppler ultrasound to detect blood vessels. In another embodiment, the one or more sensors may sense electrical measurement of nerve traffic that corresponds to an efficacy of the treatment. In another embodiment, the one or more sensors may include a vision system for direct observation. In yet another embodiment, the one or more sensors may include a force transducer, strain gauge, or similar sensor to measure radial force in the lung airways. One or more feedback mechanisms (e.g., PID, fuzzy logic, or the like) may be utilized to control the intensity of microwave energy applied, and thus the extent of damage to lung tissues and lung nerves. In some embodiments, IR measurement, tissue optical parameter measurement (e.g., reflectance, color, scattering), direct temperature measurement (e.g., using thermocouples), or other suitable mechanisms may be utilized to measure the temperature change of lung tissue in response to the applied microwave energy.

Microwave ablation may utilize dielectric hysteresis to produce heat. Tissue destruction may occur when tissues are heated to lethal temperatures from an applied electromagnetic field of, e.g., 900-2500 MHz, although other suitable ranges are also contemplated. Polar molecules in tissue (e.g., water) may be forced to continuously realign with the oscillating electric field, increasing their kinetic energy and the temperature of the tissue. Tissues with a high percentage of water may be the most conducive to microwave energy treatments.

Microwave energy may radiate into the tissue via an energy delivery element (e.g., a microwave antenna) which may function to couple energy from a generator power source to the tissues of the body. Due to the radiating nature of the energy delivery element, direct heating may occur in a volume of tissue surrounding the energy delivery element. The mechanism of heating may differ from RF ablation, which may create heat via resistive type heating when electrical current passes through an ionic tissue medium.

Microwave energy may be capable of propagating through, and effectively heating many types of tissue, including those tissues with low electrical conductivity, high impedance, or low thermal conductivity. For example, in at least some examples, bone and lung tissue may be associated with suboptimal outcomes or local progression during RF ablation due to high baseline impedance. One potential advantage of microwave energy applications may be that treatment times may be substantially shorter than RF energy treatment times. Other advantages may include that operation and outcome may not depend on electrical contact, and that operation may be less affected by tissue hydration. In at least some examples, microwave energy applications may be more controllable and more predictable for use in lung airway applications. In some examples, sensory nerves may be highly sensitive to microwave treatments, resulting in neuritis and neuropathy.

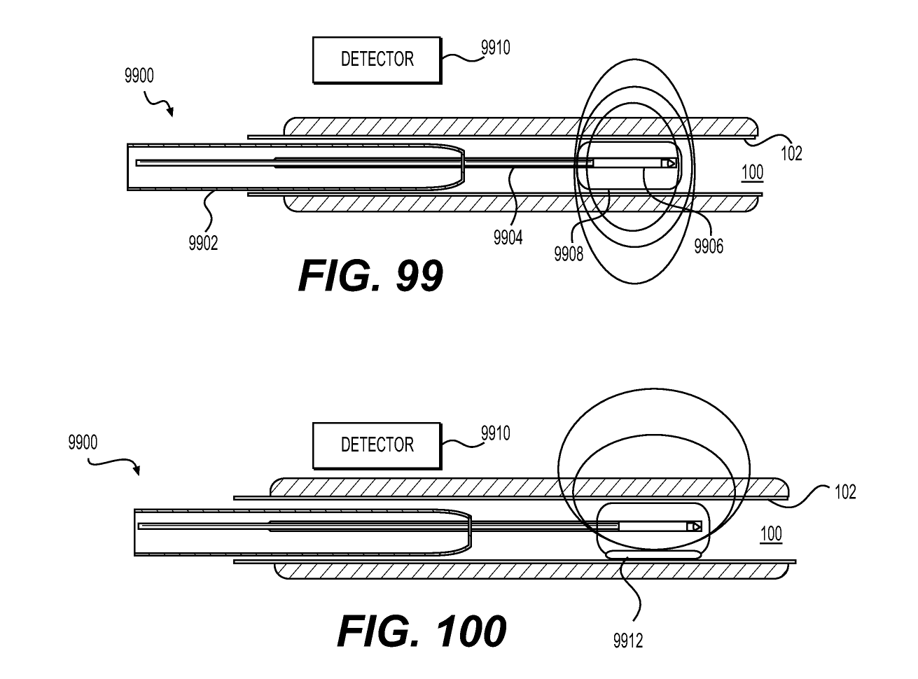

A medical device 9900 is shown in FIG. 99 disposed within an airway 100. Medical device 9900 may include a first elongate member 9902, such as, e.g., a bronchoscope. A second elongate member 9904 may extend from the distal end of first elongate member 9902. An energy delivery element 9906 (e.g., a microwave antenna) may be disposed at a distal end of second elongate member 9908. Energy delivery element 9906 may be coupled to a generator and/or power distribution system, and may be configured to deliver microwave energy to tissues of the body as set forth above. In some examples, an energizing member, such as, e.g., a coaxial cable or wire, may transmit microwave radiation from the generator and/or power distribution system to the energy delivery element 9906. In some examples, microwave energy may be delivered at a frequency of 2.45 GHz, although other suitable frequencies are also contemplated. In other examples, the microwave energy may be delivered between 2 and 4 GHz (S Band). Microwave energy and heating may be applied simultaneously over any suitable portion of the airway 100, including over relatively longer lengths (e.g., 100 mm or more), so as to treat patients rapidly. The relatively large treatment areas permitted by microwave energy therapy may result in reduced treatment times. In some examples, energy delivery element 9906 may include an additional antenna configured to emit a low power radiation at a different frequency (e.g., 1 GHz) than an ablative or treatment energy frequency. The lower frequency may be detected by a detector positioned outside of the body, and may be indicative of the temperature of energy delivery element 9906 and/or of treated tissue.

In some examples, energy delivery element 9906 may be disposed within an expandable member 9908, such as, e.g., an inflatable balloon. Medical device 9900 may circulate a cooling fluid through one or more of expandable member 9908 and a lumen (not shown) of second elongate member 9904 to prevent overheating of second elongate member 9904 or other components of medical device 9900 by reflected power and leakage.

Energy delivery element 9906 may be positioned in a center of airway 100 by, e.g., expandable member 9908. Alternatively, energy delivery element 9906 may be positioned closer to one radial side of airway 100 than an opposing side of airway 100. In some examples, energy delivery element 9906 may be selectively positioned within airway 100 by a positioning mechanism (not shown) such as a selectively articulatable member, or other suitable mechanism.

Medical device 9900 may include one or more detectors 9910 that are configured to detect microwave energy. Detectors 9910 may function on the premise that microwave energy may be absorbed by saline at an increased rate as temperature rises. In some examples, detector 9910 may be positioned outside the body in a line of sight of the microwave energy being emitted by energy delivery element 9906. The detected power may be utilized to determine the temperature of the tissue being treated. The power detected by detector 9910 may decrease as the temperature of the tissue rises. Alternatively, detector 9910 may be positioned on or adjacent to energy delivery element 9906 to detect microwave energy that has reflected and scattered. Detector 9910 may be coupled to one or more of a signal processor, PID controller, and microwave generator to provide a real-time feedback loop for energy delivery.

In some examples, microwave energy may be applied over a uniform and fully circumferential region of airway 100. However, various choke designs may be utilized to localize the heating of energy delivery element to a distal end or distal tip of second elongate member 9904, or to a particular radial region of a body lumen or airway 100.

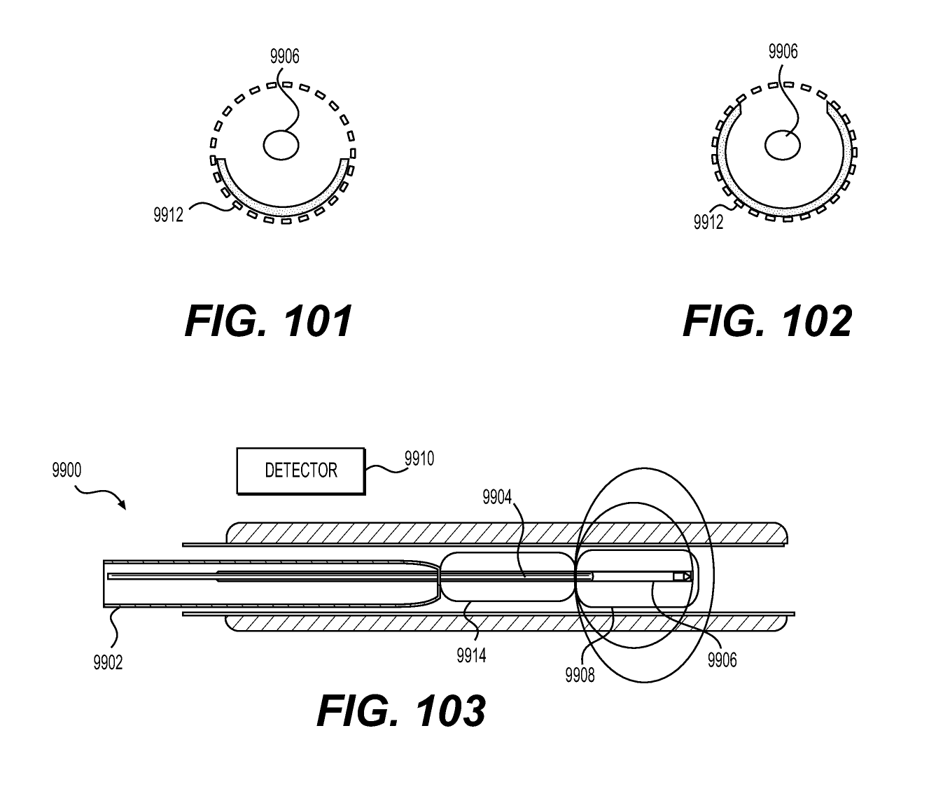

For example, referring to FIGS. 100-102, medical device 9900 may include an energy shield 9912 configured to block at least a portion of the microwave energy emitted by energy delivery element 9906 from reaching tissue. For example, energy shield 9912 may be a metal coated shield or sleeve. In some examples, energy shield 9912 may be internally-cooled via suitable lumens and cooling fluids, if desired. FIG. 101 shows an example where energy shield 9912 is configured to prevent at least half of the circumferential area of airway 100 from receiving microwave energy emitted by energy delivery element 9906. FIG. 101 shows another example where energy shield 9912 is configured to prevent more than half of the circumferential area of airway 100 from receiving microwave energy emitted by energy delivery element 9906. However, it is contemplated that energy shield may encompass other suitable sizes and ranges to allow energy delivery element 9906 to deliver microwave energy to the circumference of an airway between 0 and 360 degrees of the airway. Additionally, energy shield 9912 may take other suitable shapes and configurations to allow for treatment of airway 100 in various patterns, such as, e.g., spiral, circumferential, linear, zig-zag, among others. For example, to deliver a spiral treatment pattern to surface tissue 102, a hollow cylindrical energy shield 9912 may include one or more spiral shaped cutouts extending along a longitudinal axis such that all microwave energy emitted by energy delivery element 9906 may be prevented from reaching and ablating tissue, except for the energy that can escape through the spiral or other suitably shaped cutouts. It is also contemplated that energy shield 9912 may be curved to focus microwave energy at known distances, or to create a beam with lower divergence. The shape of the energy shield 9912 may be circular (focusing), elliptical (focusing), or parabolic (beam forming or focusing).

Medical device 9900 is shown in FIG. 103 with a second expandable member 9914 disposed on second elongate member 9904. The second expandable member 9914 may be disposed proximal to expandable member 9908, and may be a fluid filled inflatable member (e.g., balloon) that is configured to shield and limit temperature rise in tissues adjacent to or proximal to the energy delivery element 9906. For example, expandable member 9908 may be configured to prevent teardrop heating formations that are common in microwave heating applications. It is also contemplated that medical device 9900 may include a second expandable member 9914 that is distal to expandable member 9908. In some examples, expandable member 9908 may be filled with a fluid such as, e.g., hypertonic saline. Hypertonic saline may have improved microwave energy absorption properties compared to saline or water.

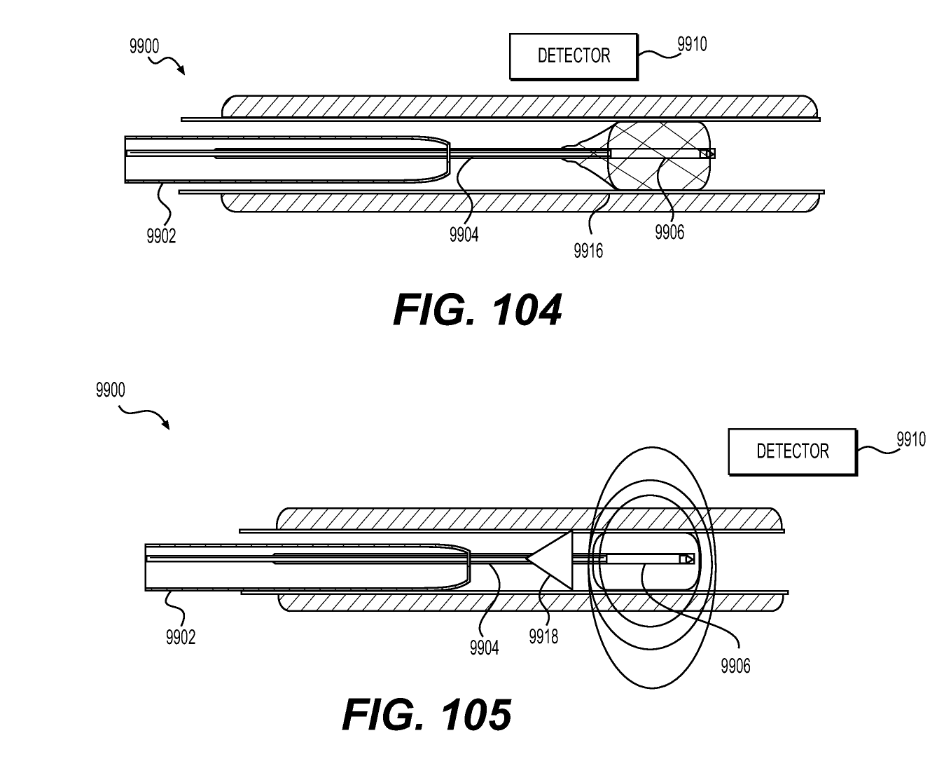

Medical device 9900 is shown in FIG. 104 with an expandable member 9916 instead of expandable member 9908, that is configured to position energy delivery element 9906 within airway 100. Expandable member 9916 may be a stent or basket movable between a collapsed configuration and an expanded configuration.

Medical device 9900 is shown in FIG. 105 with a second expandable member 9918 disposed on second elongate member 9904. The second expandable member 9918 may be disposed proximal to expandable member 9908, and may be an energy shield that is configured to shield and limit temperature rise in tissues adjacent to or proximal to the energy delivery element 9906. Second expandable member 9918 may be reciprocally movable between a collapsed configuration and an expanded configuration, and may function in a substantially similar manner as energy shield 9912. In one example, second expandable member 9918 may include metal foil disposed over a nitinol basket structure. It is further contemplated that medical device 9900 also may include a second expandable member 9918 that is distal to expandable member 9908.

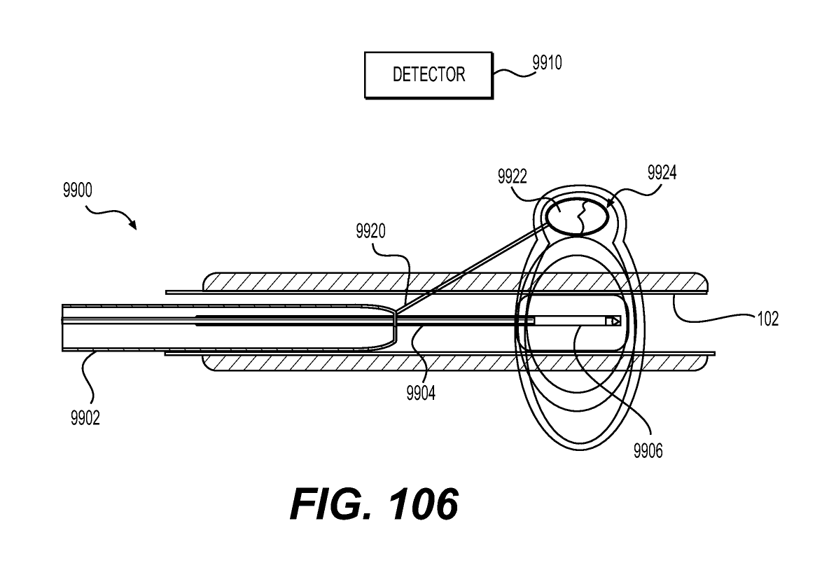

Medical device 9900 is shown in FIG. 106 with a fluid delivery device 9920 that may extend from the distal end of first elongate member 9902. Fluid delivery device 9920 may be a syringe, needle, or other suitable fluid delivery device capable of delivering fluid through airway wall 102. Fluid delivery device 9920 may pierce through airway wall 102 to deliver fluid to a target location 9922, such as, e.g., a nerve or tumor. Fluid delivery device 9920 may deliver fluid to target location 9922 such that a formation 9924, or bolus of fluid surrounds the target location 9922. In one example, the fluid may be hypertonic saline which may absorb microwave energy at a higher rate than the surrounding tissues, thus specifically treating the target location 9922. Alternatively, formation 9924 may include a plurality of small sacs of fluid, or may be a gel formation to allow the formation 9924 to remain in the body for a longer period of time. In another example, the fluid may include one or more therapeutic agents. The therapeutic agents may be, e.g., anti-cancer agents, or may contain PLGA particles containing anti-cancer agents. The formation 9924 then may serve to focus microwave energy delivery at the target location, in addition to therapeutically treating the target location.

Any aspect set forth in any embodiment may be used with any other embodiment set forth herein. The devices and apparatus set forth herein may be used in any suitable medical procedure, and may be advanced through any suitable body lumen and body cavity. For example, the apparatuses and methods described herein may be used through any natural body lumen or tract, or through incisions in any suitable tissue.

It will be apparent to those skilled in the art that various modifications and variations can be made in the disclosed systems and processes without departing from the scope of the invention. Other embodiments of the invention will be apparent to those skilled in the art from consideration of the specification and practice of the invention disclosed herein. It is intended that the specification and examples be considered as exemplary only. The following disclosure identifies some other exemplary embodiments.

1-20. (canceled)

21. A medical device for treating tissue, the medical device comprising:
an expandable member configured to be disposed in a body lumen and move between a collapsed configuration and an expanded configuration via the delivery of a fluid to the expandable member, wherein the expandable member permits the passage of substances through the body lumen from proximal of the expandable member to distally of the expandable member while the expandable member is in the expanded configuration; and
one or more energy delivery elements coupled to the expandable member, the one or more energy delivery elements being configured to damage tissues disposed radially outward of the lumen.

22. The medical device of claim 21, wherein the expandable member includes a plurality of radially outermost portions that are configured to cause one or more energy delivery elements to contact tissue when the expandable member is in the expanded configuration, wherein the plurality of radially outermost portions are circumferentially spaced from one another.

23. The medical device of claim 22, wherein the one or more energy delivery elements are disposed on the plurality of radially outermost portions.

24. The medical device of claim 23, wherein each of the plurality of radially outermost portions includes an energy delivery element.

25. The medical device of claim 23, further including a plurality of energy delivery elements arranged in a spiral about the plurality of radially outermost portions.

26. The medical device of claim 23, wherein the expandable member has a x-shaped cross-section.

27. The medical device of claim 21, wherein the expandable member includes an inner lumen that permits the passage of substances disposed within the body lumen through the expandable member when the expandable member is in the expanded configuration.

28. The medical device of claim 27, wherein the expandable member has a ring-shaped cross-section.

29. The medical device of claim 28, wherein an outer surface of the expandable member is configured to contact an entire circumference of a body lumen while in the expanded configuration.

30. The medical device of claim 21, wherein the expandable member is a balloon.

31. A medical device for treating tissue, the medical device comprising:
an expandable member configured to move between a collapsed configuration and an expanded configuration, the expandable member including a plurality of expandable legs that each extends from a proximal end toward a distal end in a spiral configuration, each of the expandable legs having one or more radially outermost portions, wherein each of the plurality of expandable legs extends along a different trajectory such that each of the radially outermost portions of the medical device are longitudinally and circumferentially staggered from one another; and
a plurality of energy delivery elements coupled to the expandable member and configured to damage tissues disposed radially outward of the lumen, wherein each of the plurality of energy delivery elements is positioned at a radially outermost portion of a corresponding one of the plurality of expandable legs.

32. The medical device of claim 31, wherein the distal ends of the plurality of expandable legs converge toward one another at a distal end.

33. The medical device of claim 31, wherein the distal ends of the plurality of expandable legs are unconnected to one another.

34. The medical device of claim 31, wherein each of the plurality of energy delivery elements includes a lumen, and wherein the medical device is configured to circulate a cooling fluid through the lumen of each of the plurality of energy delivery elements.

35. A medical device for treating tissue, the medical device comprising:
an expandable member configured to move between a collapsed configuration, a partially-expanded configuration, and an expanded configuration via the delivery of a fluid to the expandable member; and
an energy delivery element coupled to the expandable member, the energy delivery element including a bladed tip configured to apply a pressure to the surface of tissue when the medical device is in the expanded configuration, wherein the energy delivery element is disposed on an outer surface of the expandable member at a first portion, wherein the first portion of the outer surface is disposed closer to a radial center of the expandable member than a remaining portion of the outer surface when the expandable member is in the partially-expanded configuration.

36. The medical device of claim 35, wherein the energy delivery element includes two inclined surfaces that meet at the bladed tip.

37. The medical device of claim 36, wherein each of the inclined surfaces includes an outlet in fluid communication with an interior of the expandable member.

38. The medical device of claim 35, wherein the bladed tip includes an outlet in fluid communication with an interior of the expandable member.

39. The medical device of claim 35, wherein the expandable member is a balloon.

40. The medical device of claim 35, wherein the energy delivery element is configured to deliver radiofrequency energy.
